(19) United States
(12) Patent Application Publication
(10) Pub. No.: US 2007/0244088 A1
(43) Pub. Date: Oct. 18, 2007

(54) New Pyridine Analogues II

(75) Inventors: Kay Brickmann, Molndal (SE); Fredrik Zetterberg, Molndal (SE)

Correspondence Address: Pepper Hamilton LLP, 500 Grant Street, One Mellon Bank Center, 50th Floor, Pittsburgh, PA, 15219-2502, US

(73) Assignee: ASTRAZENECA AB, Sodertalje (SE)

(21) Appl. No.: 11/768,372

(22) Filed: Jun. 26, 2007

(30) Foreign Application Priority Data
Jul. 4, 2006 (SE) 0601467-4
Jun. 28, 2004 (SE) 0601412-0

(51) Int. Cl. A61K 31/551; A61K 31/496; A61K 31/4439; C07D 413/02; C07D 403/02

(52) U.S. Cl. 514/210.2; 514/218; 514/253.13; 514/340; 540/575; 544/360; 546/268.4

(57) ABSTRACT

The present invention relates to certain new pyridin analogues of Formula (I) 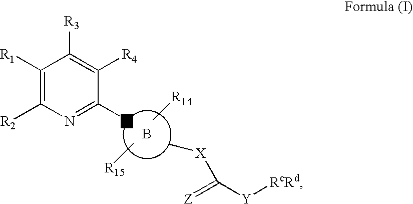
to processes for preparing such compounds, to their utility in medicine in general and especially as P2Y₁₂ inhibitors and as anti-thrombotic agents, etc, their use as medicaments in cardiovascular diseases as well as pharmaceutical compositions containing them.

### CROSS-REFERENCE TO RELATED APPLICATIONS

This application claims priority to Swedish Application No. 0601467-4 filed Jul. 4, 2006 and to Swedish Application No. 0601412-0 filed Jun. 28, 2006, each which is incorporated herein by reference in its entirety.

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists'Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit of Clopidogrel treatment is associated with an increased rate of clinical bleeding. Published data suggest that reversible P2Y₁₂ antagonists could offer the possibility for high clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists.)

Accordingly it is an object of the present invention to provide potent, reversible and selective P2Y₁₂-antagonists as anti-thrombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below. Examples of such beneficial properties are high potency, high selectivity, and an advantageous therapeutic window.

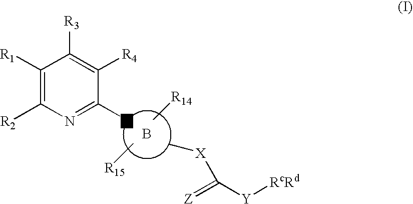

### DESCRIPTION OF EMBODIMENTS

According to the present invention there is provided a novel compound of formula (I) or a pharmaceutically acceptable salt thereof: 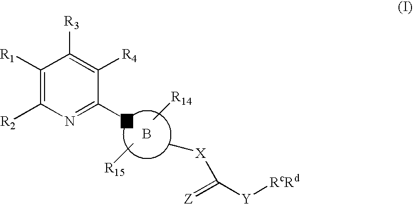
wherein:

R₁ represents R₆OC(O), R₇C(O), R₁₆SC(O), R₁₇S, R₁₈C(S) or a group gII

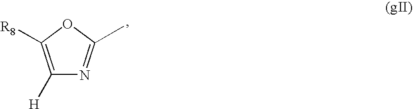

preferably R₁ represents R₆OC(O) or the group gII;

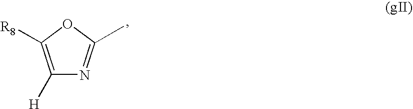

R₂ represents H, CN, halogen (F, Cl, Br, I), NO₂, (C₁-C₁₂)alkyl optionally interrupted by oxygen and/or optionally substituted by OH, aryl, cycloalkyl, heterocyclyl or one or more halogen (F, Cl, Br, I) atoms; further R₂ represents (C₁-C₁₂)alkoxy optionally substituted by one or more halogen (F, Cl, Br, I) atoms; further R₂ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine;

Further, R₁+R₂ together (with two carbon atoms of the pyridine ring) may form a 5-membered or 6-membered cyclic lactone;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents a halogen atom (F, Cl, Br, I) or is CN;

Z represents O (oxygen) or S (sulphur);

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R₁₇ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;

Y represents imino (—NH—) or is absent;

R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;

R₁₉ represents H or (C₁-C₄)alkyl;

R^{d} represents (C₁-C₁₂)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, aryloxy, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections);

with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

Preferred values of each variable group are as follows. Such values may be used where appropriate with any of the values, definitions, claims, aspects or embodiments defined hereinbefore or hereinafter. In particular, each may be used as an individual limitation on the broadest definition as well as any other of the embodiments of formula (I).

For the avoidance of doubt it is to be understood that where in this specification a group is qualified by “hereinbefore defined”, “defined hereinbefore” or “defined above” the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkyl” includes both linear or branched chain groups, optionally substituted by one or more halogens (F, Cl, Br, I) or mixed halogen atoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, optionally substituted by one or more halogens (F, Cl, Br, I) or mixed halogen atoms.

The term “aryl” denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, halogen substituted (C₁-C₁₂)alkoxy, (C₃-C₆)cycloalkyl, aryl, aryloxy, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic heterocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzodioxol, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In another embodiment R₁ represents a group (gII),

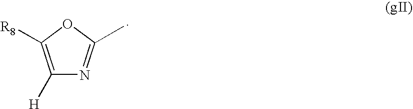

In a further embodiment of the invention R₁ is R₆OC(O) wherein R₆ can be (C₁-C₆)alkyl.

R₁ may also be embodified by the group gII, 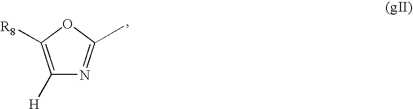
in which R₈ is selected from H, (C₁-C₆)alkyl, such as methyl or ethyl.

In another embodiment for the group R₈ this group can be chosen among hydrogen, methyl, ethyl, n-propyl and n-butyl.

Embodiments for R₂ include, for example, H and (C₁-C₄)alkyl. Other embodiments for R₂ are methyl, ethyl, iso-propyl, phenyl, methoxy, or amino unsubstituted or optionally substituted with methyl.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

Embodiments for R₄ include H, halogen such as chloro or bromo, methyl, cyano, nitro, amino unsubstituted or optionally substituted with one or two methyl groups and further includes 4-methoxy-4-oxobutoxy, 3-carboxy-propoxy and methylcarbonyl.

In one embodiment of the invention R₄ is a halogen atom (F, Cl, Br, I) or is CN.

In another embodiment of the invention R₄ is a halogen atom (F, Cl, Br, I).

In a further embodiment of the invention R₄ is CN.

In another further embodiment of the invention R₄ is CN or Cl.

In an even further embodiment of the invention R₄ is Cl.

In one embodiment of the invention Z represents S (sulphur).

In another embodiment of the invention Z represents O (oxygen).

Further embodiments for R₈ include hydrogen, methyl and ethyl.

In a special embodiment R₈ is ethyl.

Further embodiments for R₁₄ include, for example, hydrogen, methyl, amino, tert-butyloxycarbonyl, tert-butyloxycarbonyl-imino, 2-carboxyethyl and 3-tert-butoxy-3-oxo-propyl.

In one embodiment R₁₄ is hydrogen or 2-carboxyethyl.

Other further embodiments for R₁₄ include, for example, hydrogen, methyl, tert-butyloxycarbonyl-imino, and amino.

In one embodiment of the invention R₁₅ represents H.

In one embodiment of the invention X represents a single bond, imino (—NH—) or iminomethylene (—CH₂—NH—).

In one embodiment of the invention Y is absent.

In another embodiment of the invention Y is imino (—NH—).

Further embodiments for R^{d} includes aryl or heterocyclyl, more particularly, aryl or aromatic heterocyclyl.

In another further embodiment R^{d} is alkyl, cycloalkyl or aryl.

Another embodiment for R^{d} include, aryl such as phenyl and aromatic heterocyclyl such as thienyl.

In one other embodiment R^{d} is phenyl or cyclopropyl, which either one optionally may be substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, aryloxy, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Even further embodiments for R^{d} include phenyl optionally substituted at the 2, 3, 4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₄)alkylene group with any substituents according to above.

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₃)alkylene group with any substituents according to above.

In a special embodiment R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine.

In a further embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₄)alkylene group with any substituents according to above.

In a further preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₃)alkylene group with any substituents according to above.

In a particular embodiment of the invention R^{c} represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-C₁-alkylene group with any substituents according to above.

In a further particular embodiment R^{c} represents imino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine.

In a utterly further particular embodiment R^{c} represents imino or (C₁-C₄)alkyleneimino or (C₁-C₄)oxoalkylene group.

In a utterly further particular special embodiment R^{c} represents imino or (C₁-C₄)alkyleneimino or an unsubstituted or monosubstituted or polysubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine.

In one embodiment of the invention R^{c} is absent.

In one embodiment of the invention R₁₉ represents hydrogen.

In a further embodiment of the invention R₁₉ represents (C₁-C₄)alkyl.

In another embodiment of the invention R₁₉ represents hydrogen or methyl.

In a particular embodiment of the invention R₁₉ represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents imino (—NH—) or methylene (—CH₂—).

In yet another embodiment X represents imino (—NH—).

In a further embodiment X represents methylene (—CH₂—).

Suitable values for the B ring/ring system include, for example, diazepanylene, piperazinylene, piperidinylene, pyrrolidinylene and azetidinylene, wherein anyone of them may be presents in any of their isomeric forms (e.g. piperazin-tetrahydropyridazin-tetrahydropyrimidin).

Embodiments for the B ring/ring system include, for example, diazepanylene, piperazinylene, piperidinylene, pyrrolidinylene and azetidinylene. Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

In a preferred special embodiment the following combination of variable groups is defined as follows, and may be combined with the other variable groups of formula I according to any given embodiment of the invention (e.g. the one defined above or in the “2^{nd} embodiment” or “3^{rd} embodiment”);

R₁ is R₆OC(O), Z is O (oxygen), and X represents imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl, and Y represents imino (—NH—) or is absent.

In a second preferred special embodiment the following combination of variable groups is defined as follows, and may be combined with the other variable groups of formula I according to any given embodiment of the invention (e.g. the one defined above or in the “2^{nd} embodiment” or “3^{rd} embodiment”);

R₁ represents R₇C(O), R₁₆SC(O), R₁₇S, R₁₈C(S) or a group gII,

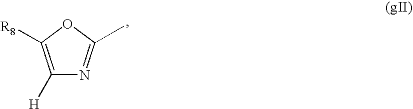

Z is O (oxygen), X represents a single bond, and Y represents imino (—NH—) or is absent.

A 2nd embodiment of formula I is defined by:

R₁ represents R₆OC(O), R₇C(O), R₁₆SC(O), R₁₇S, R₁₈C(S) or a group gII,

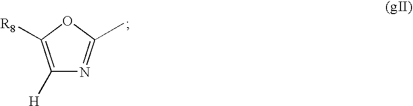

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further R₂ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

Further, R₁+R₂ together (with two carbons from the pyridine ring) may form a 5-membered or 6-membered cyclic lactone;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents a halogen atom (F, Cl, Br, I) or is CN;

Z represents O (oxygen) or S (sulphur);

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;

R₁₇ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;

Y represents imino (—CH₂—) or is absent;

R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;

R₁₉ represents H or (C₁-C₄)alkyl;

R^{d} represents (C₁-C₁₀)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, aryloxy, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections);

with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

A 3rd embodiment of formula I is defined by:

R₁ represents R₆OC(O) or a group gII,

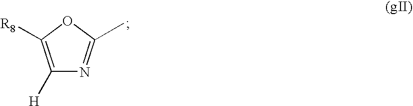

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further R₂ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents a halogen atom (F, Cl, Br, I) or is CN;

Z represents O (oxygen) or S (sulphur);

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ is ethyl;

R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;

R₁₉ represents H or (C₁-C₄)alkyl;

R^{d} represents (C₁-C₁₀)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxy, halosubstituted (C₁-C₆)alkyl, (C₃-C₆)cycloalkyl, aryl, aryloxy, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂—)ₙ wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections);

with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

A 4th embodiment of formula I is defined by:

R₁ represents R₆OC(O) or a group gII

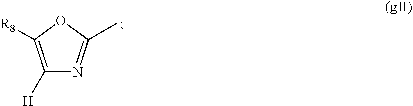

R₂ represents H or (C₁-C₆)alkyl optionally interrupted by oxygen and/or optionally substituted by OH, aryl, cycloalkyl, heterocyclyl or one or more halogen (F, Cl, Br, I) atoms;

R₃ represents H;

R₄ represents CN or halogen (F, Cl, Br, I);

Z represents O (oxygen) or S (sulphur);

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms;

R₈ represents H, (C₁-C₆)alkyl optionally interrupted by oxygen, and/or optionally substituted by aryl, cycloalkyl, heterocyclyl or one or more halogen (F, Cl, Br, I) atoms;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl;

R₁₅ represents H;

R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;

R^{d} represents (C₁-C₁₀)alkyl, (C₃-C₆)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, (C₁-C₆)alkylthio, halosubstituted (C₁-C₆)alkyl, aryl and aryloxy;

X represents a single bond, imino (—NH—), methylene (—CH₂—) or iminomethylene (—CH₂—NH—); and

B is a monocyclic, 4 to 7-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections);

with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

A 5th embodiment of formula I is defined by that:

R₁ is ethoxycarbonyl;

R₂ is chosen from a group consisting of H, methyl and trifluoromethyl;

R₃ is H;

R₄ is chosen from a group consisting of bromo, chloro and cyano;

Z represents O (oxygen) or S (sulphur);

R₅ is H;

R₆ is ethyl;

R₈ is ethyl;

R₁₄ is chosen from a group consisting of H and carboxyethyl;

R₁₅ is H;

R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;

R^{d} is chosen from a group consisting of n-octyl, 2-phenyl-cyclopropyl, phenyl, 2-methylphenyl, 3-methoxycarbonyl-phenyl, 2-methoxy-5-methyl-phenyl, 4-methoxy-2-methyl-phenyl, 3-methylphenyl, 4-methylphenyl, 2-methoxyphenyl, 3-methoxyphenyl, 4-methoxyphenyl, 4-butoxy-phenyl, 2,6-dimethoxy-phenyl, 3-thiomethyl-phenyl, 4-thiomethyl-phenyl, 2-ethyl-6-isopropyl-phenyl, 2-fluoro-5-methyl-phenyl, 3-fluoro-5-(trifluoromethyl)-phenyl, 3-fluorophenyl, 4-fluorophenyl, 4-fluoro-3-nitro-phenyl, 3,4-difluorophenyl, (difluoromethoxy)-phenyl, 2-chlorophenyl, 3-chlorophenyl, 4-chlorophenyl, 5-chloro-2,4-dimethoxy-phenyl, 2-bromophenyl, 3-bromophenyl, 4-bromophenyl, 3-cyanophenyl, 2-ethoxyphenyl, 4-ethoxyphenyl, 3-nitrophenyl, 2-methyl-3-nitrophenyl, 3,5-dinitrophenyl, 2,4-dichlorophenyl, 3,4-dichlorophenyl, 3,5-dichlorophenyl, 2,4,5-trichloro-phenyl, 4,5-dimethyl-2-nitro-phenyl, 4-(dimethylamino)-phenyl, 2-isopropylphenyl, 4-isopropylphenyl, 3-isopropenylphenyl, 2-phenyl-phenyl, 4-phenoxy-phenyl, 2-naphtyl, 3-naphtyl, 2-thienyl, 5-chloro-2-thienyl and 1,3-benzodioxol-5-yl;

X represents a single bond, imino (—NH—), methylene (—CH₂—) or iminomethylene (—CH₂—NH—); and

B is chosen from the group consisting of 1,4-diazepan-1-ylene, 4-piperazin-1-ylene, 4-piperidin-1-ylene, 3-azetidin-1-ylene, and the substituents R₁₄ and R₁₅ are connected to the B ring/ring system, in such a way that no quarternary ammonium compounds are formed (by these connections);

with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

In a 6th embodiment of formula (I), formula (I) is defined as being any compound(s) of formula (Ia)-(Ii):

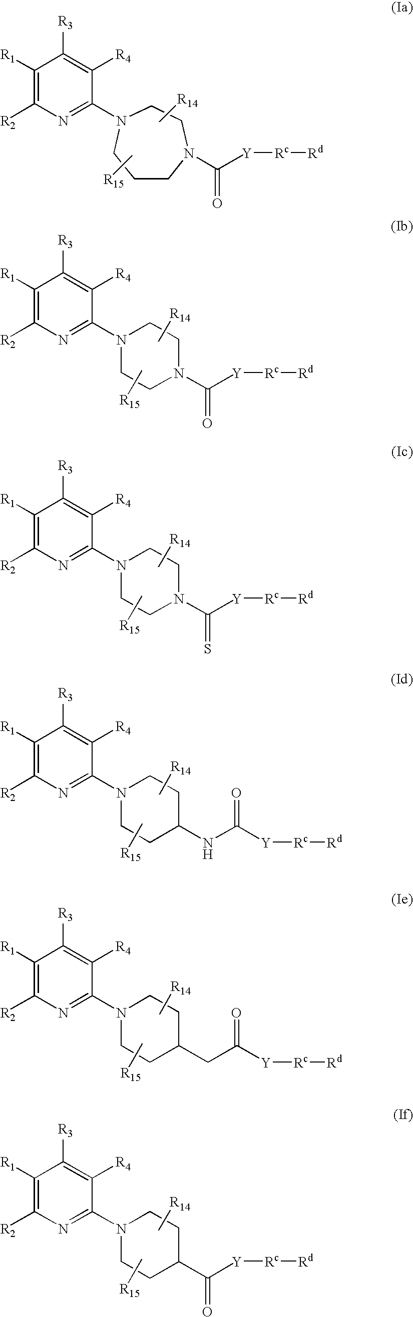

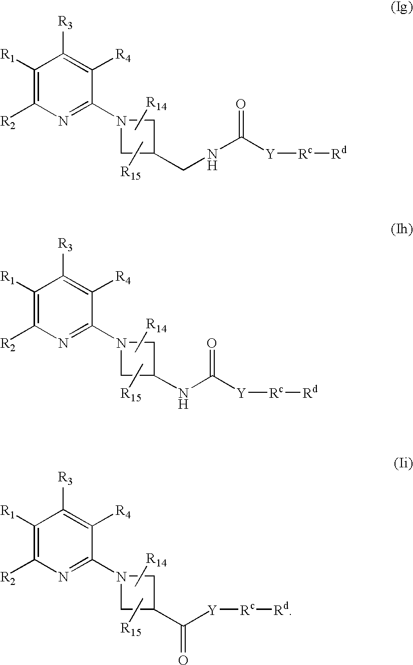

In the above Ia to Ii the various values of Y and R are as defined above and include the previously mentioned embodiments, with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

In a 7^{th} embodiment formula (I) is defined as being any compound(s) of formula (Iaa)-(Ijj):

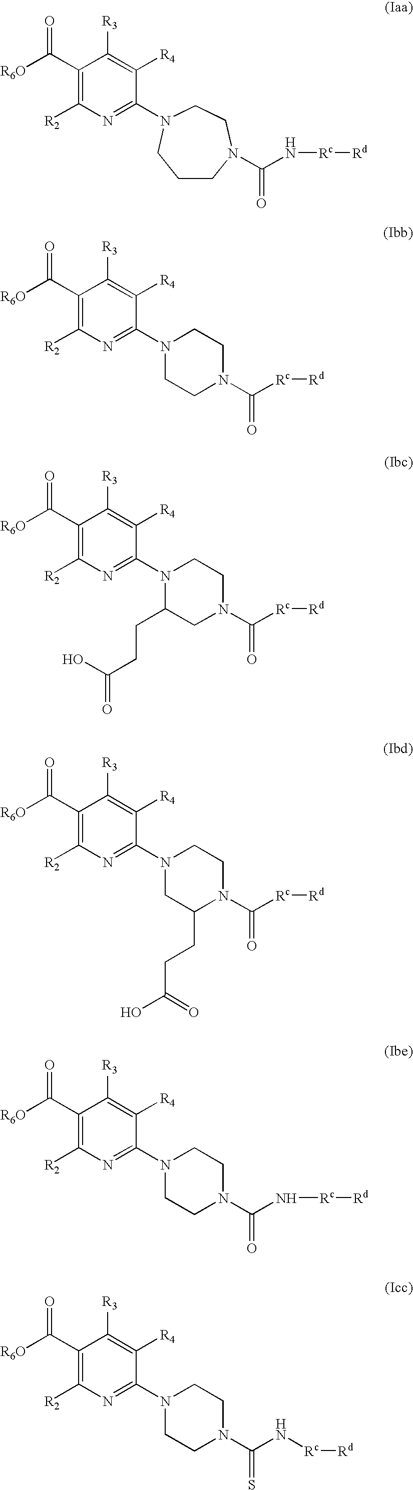

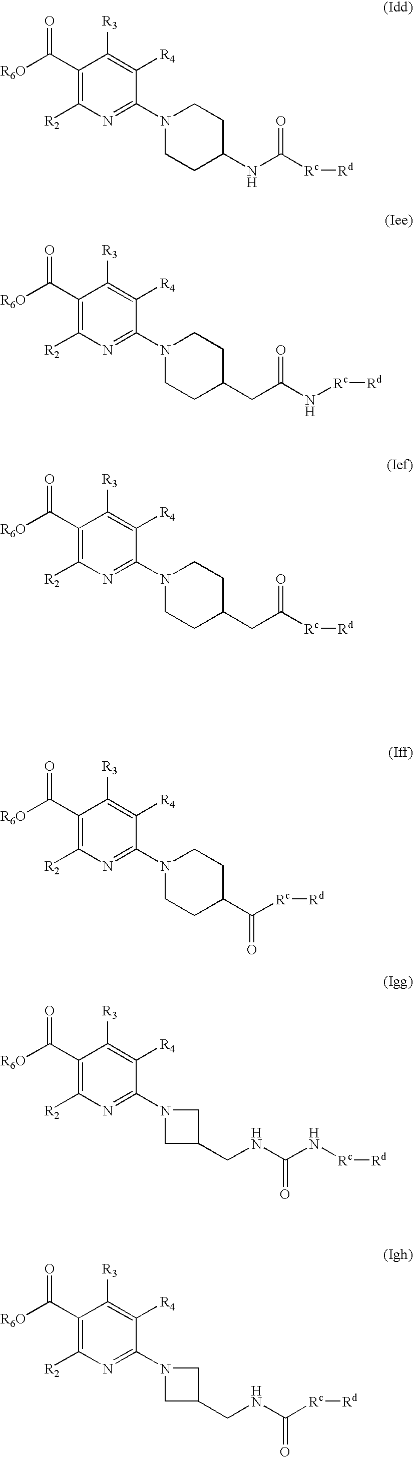

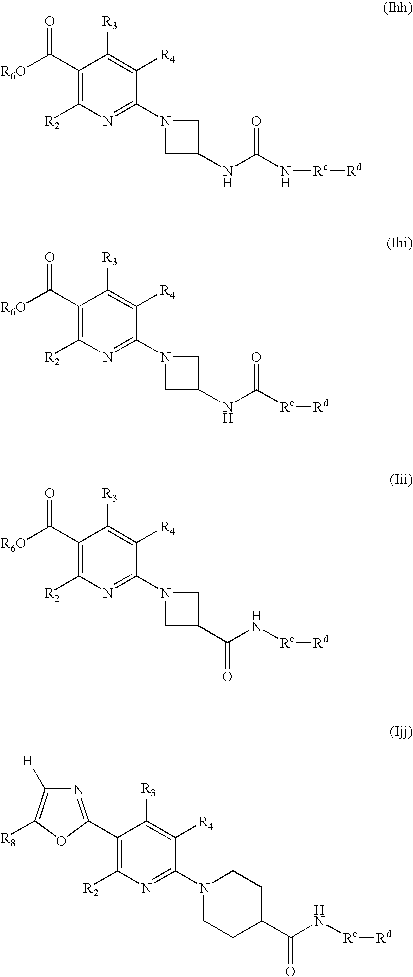

In the above Iaa to Ijj the various values of R are as defined above and include the previously mentioned embodiments, with the proviso that the compound or the pharmaceutically acceptable salt thereof is not
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

Examples of specific compounds according to the invention can be selected from:
ethyl 6-[4-(anilinocarbonyl)piperazin-1-yl]-5-chloronicotinate;
ethyl 6-[4-(anilinocarbonyl)piperazin-1-yl]-5-bromonicotinate;
3-{4-(anilinocarbonyl)-1-[3-chloro-5-(ethoxycarbonyl)pyridin-2-yl]piperazin-2-yl}propanoic acid;
ethyl 6-[4-(anilinocarbonyl)piperazin-1-yl]-5-cyanonicotinate;
ethyl 5-chloro-6-(4-{[(3,4-dichlorophenyl)amino]carbonyl}piperazin-1-yl)nicotinate;
ethyl 5-chloro-6-(4-{[(3,4-dichlorobenzyl)amino]carbonyl}piperazin-1-yl)nicotinate;
ethyl 5-chloro-6-(4-{[(2-methylbenzyl)amino]carbonyl}piperazin-1-yl)nicotinate;
ethyl 5-chloro-6-(4-{[(4-fluorobenzyl)amino]carbonyl}piperazin-1-yl)nicotinate;
ethyl 5-chloro-6-(4-{[(3-methylbenzyl)amino]carbonyl}piperazin-1-yl)nicotinate;
ethyl 5-chloro-6-(4-{[(4-methylbenzyl)amino]carbonyl}piperazin-1-yl)nicotinate;
ethyl 5-chloro-6-(4-{[(3-methoxyphenyl)amino]carbonyl}piperazin-1-yl)nicotinate;
ethyl 5-chloro-6-{4-[(2-naphthylamino)carbonyl]piperazin-1-yl}nicotinate;
ethyl 6-(4-{[(3-bromophenyl)amino]carbonyl}piperazin-1-yl)-5-chloronicotinate;
ethyl 5-chloro-6-[4-({[4-(methylthio)phenyl]amino}carbonyl)piperazin-1-yl]nicotinate;
ethyl 5-chloro-6-[4-({[3-(methylthio)phenyl]amino}carbonyl)piperazin-1-yl]nicotinate;
ethyl 5-chloro-6-(4-{[(3,5-dinitrophenyl)amino]carbonyl}piperazin-1-yl)nicotinate;
ethyl 5-chloro-6-(4-{[(2-methoxy-5-methylphenyl)amino]carbonyl}piperazin-1-yl)nicotinate;
ethyl 5-chloro-6-(4-{[(3-methylphenyl)amino]carbonyl}piperazin-1-yl)nicotinate;
ethyl 5-chloro-6-(4-{[(4-chlorophenyl)amino]carbonyl}piperazin-1-yl)nicotinate;
ethyl 5-chloro-6-(4-{[(3,5-dichlorophenyl)amino]carbonyl}piperazin-1-yl)nicotinate;
ethyl 5-chloro-6-(4-{[(2-isopropylphenyl)amino]carbonyl}piperazin-1-yl)nicotinate;
ethyl 5-chloro-6-[4-({[(1S)-1-phenylethyl]amino}carbonyl)piperazin-1-yl]nicotinate;
ethyl 5-chloro-6-[4-({[(1S)-1-(1-naphthyl)ethyl]amino}carbonyl)piperazin-1-yl]nicotinate;
ethyl 5-chloro-6-{4-[(1-naphthylamino)carbonyl]piperazin-1-yl}nicotinate;
ethyl 5-chloro-6-(4-{[(4-methylphenyl)amino]carbonyl}piperazin-1-yl)nicotinate;
ethyl 5-chloro-6-(4-{[(2-methylphenyl)amino]carbonyl}piperazin-1-yl)nicotinate;
ethyl 5-cyano-6-(4-{[(2,6-dimethoxyphenyl)amino]carbonyl}piperazin-1-yl)-2-(trifluoromethyl)nicotinate;
ethyl 5-cyano-6-(4-{[(2-methoxy-5-methylphenyl)amino]carbonyl}piperazin-1-yl)-2-(trifluoromethyl)nicotinate;
ethyl 5-cyano-6-(4-{[(2-isopropylphenyl)amino]carbonyl}piperazin-1-yl)-2-(trifluoromethyl)nicotinate;
ethyl 5-cyano-6-(4-{[(4-methylphenyl)amino]carbonyl}piperazin-1-yl)-2-(trifluoromethyl)nicotinate;
ethyl 5-cyano-6-(4-{[(3-methylphenyl)amino]carbonyl}piperazin-1-yl)-2-(trifluoromethyl)nicotinate;
ethyl 5-cyano-6-[4-({[(1S)-1-phenylethyl]amino}carbonyl)piperazin-1-yl]-2-(trifluoromethyl)nicotinate;
ethyl 5-cyano-6-(4-{[(2-ethoxyphenyl)amino]carbonyl}piperazin-1-yl)-2-(trifluoromethyl)nicotinate;
ethyl 6-(4-{[(2-chlorophenyl)amino]carbonyl}piperazin-1-yl)-5-cyano-2-(trifluoromethyl)nicotinate;
ethyl 5-cyano-6-(4-{[(2-methylbenzyl)amino]carbonyl}piperazin-1-yl)-2-(trifluoromethyl)nicotinate;
ethyl 6-(4-{[(2-chlorobenzyl)amino]carbonyl}piperazin-1-yl)-5-cyano-2-(trifluoromethyl)nicotinate;
ethyl 5-cyano-6-(4-{[(4-fluorobenzyl)amino]carbonyl}piperazin-1-yl)-2-(trifluoromethyl)nicotinate;
ethyl 5-cyano-6-[4-({[(1R,2R)-2-phenylcyclopropyl]amino}carbonyl)piperazin-1-yl]-2-(trifluoromethyl)nicotinate;
ethyl 5-cyano-6-(4-{[(3-methylbenzyl)amino]carbonyl}piperazin-1-yl)-2-(trifluoromethyl)nicotinate;
ethyl 5-cyano-6-(4-{[(4-methylbenzyl)amino]carbonyl}piperazin-1-yl)-2-(trifluoromethyl)nicotinate;
ethyl 5-cyano-6-(4-{[(3,4-dichlorobenzyl)amino]carbonyl}piperazin-1-yl)-2-(trifluoromethyl)nicotinate;
ethyl 5-cyano-6-(4-{[(3-methoxyphenyl)amino]carbonyl}piperazin-1-yl)-2-(trifluoromethyl)nicotinate;
ethyl 5-cyano-6-(4-{[(2-fluoro-5-methylphenyl)amino]carbonyl}piperazin-1-yl)-2-(trifluoromethyl)nicotinate;
ethyl 6-(4-{[(3-chlorophenyl)amino]carbonyl}piperazin-1-yl)-5-cyano-2-(trifluoromethyl)nicotinate;
ethyl 5-cyano-6-[4-({[2-(2-thienyl)ethyl]amino}carbonyl)piperazin-1-yl]-2-(trifluoromethyl)nicotinate;
ethyl 5-cyano-6-(4-{[(3-cyanophenyl)amino]carbonyl}piperazin-1-yl)-2-(trifluoromethyl)nicotinate;
ethyl 5-cyano-6-(4-{[(2-methoxyphenyl)amino]carbonyl}piperazin-1-yl)-2-(trifluoromethyl)nicotinate;
ethyl 6-{4-[(benzylamino)carbonyl]piperazin-1-yl}-5-cyano-2-(trifluoromethyl)nicotinate
ethyl 6-(4-{[(5-chloro-2,4-dimethoxyphenyl)amino]carbonyl}piperazin-1-yl)-5-cyano-2-(trifluoromethyl)nicotinate;
ethyl 5-cyano-6-(4-{[(3-nitrophenyl)amino]carbonyl}piperazin-1-yl)-2-(trifluoromethyl)nicotinate;
ethyl 5-cyano-6-[4-({[3-fluoro-5-(trifluoromethyl)phenyl]amino}carbonyl)piperazin-1-yl]-2-(trifluoromethyl)nicotinate;
ethyl 5-cyano-6-[4-({[3-(methylthio)phenyl]amino}carbonyl)piperazin-1-yl]-2-(trifluoromethyl)nicotinate;
ethyl 5-cyano-6-(4-{[(3-fluorobenzyl)amino]carbonyl}piperazin-1-yl)-2-(trifluoromethyl)nicotinate;
ethyl 5-cyano-6-{4-[(2-naphthylamino)carbonyl]piperazin-1-yl}-2-(trifluoromethyl)nicotinate;
ethyl 6-(4-{[(3-bromophenyl)amino]carbonyl}piperazin-1-yl)-5-cyano-2-(trifluoromethyl)nicotinate;
ethyl 6-(4-{[(4-bromophenyl)amino]carbonyl}piperazin-1-yl)-5-cyano-2-(trifluoromethyl)nicotinate;
ethyl 6-(4-{[(2-bromophenyl)amino]carbonyl}piperazin-1-yl)-5-chloronicotinate;
ethyl 5-chloro-6-[4-({[1-(3-isopropenylphenyl)-1-methylethyl]amino}carbonyl)piperazin-1-yl]nicotinate;
ethyl 5-chloro-6-(4-{[(2-methyl-3-nitrophenyl)amino]carbonyl}piperazin-1-yl)nicotinate;
ethyl 5-chloro-6-{4-[(2-thienylamino)carbonyl]piperazin-1-yl}nicotinate;
ethyl 5-chloro-6-(4-{[(3-chlorophenyl)amino]carbonyl}piperazin-1-yl)nicotinate;
ethyl 5-cyano-6-(4-{[(3,5-dichlorophenyl)amino]carbonyl}piperazin-1-yl)-2-(trifluoromethyl)nicotinate;
ethyl 5-cyano-6-(4-{[(2-methyl-3-nitrophenyl)amino]carbonyl}piperazin-1-yl)-2-(trifluoromethyl)nicotinate;
ethyl 6-{4-[(biphenyl-2-ylamino)carbonyl]piperazin-1-yl}-5-cyano-2-(trifluoromethyl)nicotinate;
ethyl 5-cyano-6-(4-{[(3,4-dichlorophenyl)amino]carbonyl}piperazin-1-yl)-2-(trifluoromethyl)nicotinate;
ethyl 5-cyano-6-[4-({[1-(3-isopropenylphenyl)-1-methylethyl]amino}carbonyl)piperazin-1-yl]-2-(trifluoromethyl)nicotinate;
ethyl 5-cyano-6-(4-{[(4-phenoxyphenyl)amino]carbonyl}piperazin-1-yl)-2-(trifluoromethyl)nicotinate;
ethyl 5-cyano-6-(4-{[(4-methoxybenzyl)amino]carbonyl}piperazin-1-yl)-2-(trifluoromethyl)nicotinate;
3-{1-(anilinocarbonyl)-4-[3-cyano-5-(ethoxycarbonyl)-6-(trifluoromethyl)pyridin-2-yl]piperazin-2-yl}propanoic acid;
ethyl 6-{4-[(anilinocarbonyl)amino]piperidin-1-yl}-5-chloronicotinate;
ethyl 6-{3-[(anilinocarbonyl)amino]azetidin-1-yl}-5-chloronicotinate;
ethyl 6-(3-{[(anilinocarbonyl)amino]methyl}azetidin-1-yl)-5-cyano-2-methylnicotinate;
ethyl 6-[3-({[(benzylamino)carbonyl]amino}methyl)azetidin-1-yl]-5-cyano-2-methylnicotinate;
ethyl 6-{3-[(anilinocarbonyl)amino]azetidin-1-yl}-5-cyano-2-methylnicotinate;
ethyl 6-(3-{[(benzylamino)carbonyl]amino}azetidin-1-yl)-5-cyano-2-methylnicotinate;
ethyl 6-{4-[(benzoylamino)carbonothioyl]piperazin-1-yl}-5-chloronicotinate;
ethyl 5-cyano-2-methyl-6-(3-{[(phenylacetyl)amino]methyl}azetidin-1-yl)nicotinate;
ethyl 6-{3-[(benzoylamino)methyl]azetidin-1-yl}-5-cyano-2-methylnicotinateethyl 6-[4-(2-anilino-2-oxoethyl)piperidin-1-yl]-5-cyano-2-methylnicotinate;
ethyl 6-{4-[2-(benzylamino)-2-oxoethyl]piperidin-1-yl}-5-cyano-2-methylnicotinate;
phenylalanine, N-[[1-[3-cyano-5-(ethoxycarbonyl)-6-methyl-2-pyridinyl]-3-azetidinyl]carbonyl]-ethyl 5-chloro-6-(4-{[(2,4,5-trichlorophenyl)amino]carbonyl}piperazin-1-yl)nicotinate;
ethyl 6-{4-[(1,3-benzodioxol-5-ylamino)carbonyl]piperazin-1-yl}-5-cyano-2-(trifluoromethyl)nicotinate;
ethyl 5-cyano-6-(4-{[(4-isopropylphenyl)amino]carbonyl}piperazin-1-yl)-2-(trifluoromethyl)nicotinate;
ethyl 5-cyano-6-(4-{[(2-phenylethyl)amino]carbonyl}piperazin-1-yl)-2-(trifluoromethyl)nicotinate;
ethyl 6-{4-[(benzylamino)carbonyl]-1,4-diazepan-1-yl}-5-cyano-2-methylnicotinate;
ethyl 5-chloro-6-[4-({[(1R,2R)-2-phenylcyclopropyl]amino}carbonyl)piperazin-1-yl]nicotinate;
ethyl 5-cyano-6-(4-{[(3,4-difluorophenyl)amino]carbonyl}piperazin-1-yl)-2-(trifluoromethyl)nicotinate;
ethyl 5-cyano-6-(4-{[(2-methylphenyl)amino]carbonyl}piperazin-1-yl)-2-(trifluoromethyl)nicotinate;
ethyl 5-cyano-6-(4-{[(4-ethoxyphenyl)amino]carbonyl}piperazin-1-yl)-2-(trifluoromethyl)nicotinate;
ethyl 5-cyano-6-[4-({[4-(methylthio)phenyl]amino}carbonyl)piperazin-1-yl]-2-(trifluoromethyl)nicotinate;
ethyl 6-{4-[(1,3-benzodioxol-5-ylamino)carbonyl]piperazin-1-yl}-5-chloronicotinate;
3-{1-{[(5-chloro-2-thienyl)amino]carbonyl}-4-[3-cyano-5-(ethoxycarbonyl)-6-(trifluoromethyl)pyridin-2-yl]piperazin-2-yl}propanoic acid;
ethyl 5-chloro-6-(4-{[(2,4-dichlorophenyl)amino]carbonyl}piperazin-1-yl)nicotinate;
ethyl 5-chloro-6-(4-{[(3-nitrophenyl)amino]carbonyl}piperazin-1-yl)nicotinate;
ethyl 5-cyano-6-(4-{[(4-fluoro-3-nitrophenyl)amino]carbonyl}piperazin-1-yl)-2-(trifluoromethyl)nicotinate;
ethyl 5-cyano-6-[4-({[4-(dimethylamino)phenyl]amino}carbonyl)piperazin-1-yl]-2-(trifluoromethyl)nicotinate;
ethyl 5-chloro-6-(4-{[(4,5-dimethyl-2-nitrophenyl)amino]carbonyl}piperazin-1-yl)nicotinate;
ethyl 5-cyano-6-(4-{[(4-methoxy-2-methylphenyl)amino]carbonyl}piperazin-1-yl)-2-(trifluoromethyl)nicotinate;
ethyl 5-chloro-6-(4-{[(2-methoxyphenyl)amino]carbonyl}piperazin-1-yl)nicotinate;
ethyl 6-(4-{[(4-butoxyphenyl)amino]carbonyl}piperazin-1-yl)-5-chloronicotinate;
ethyl 6-{4-[(benzylamino)carbonyl]piperazin-1-yl}-5-chloronicotinate;
ethyl 5-cyano-6-{4-[(octylamino)carbonyl]piperazin-1-yl}-2-(trifluoromethyl)nicotinate;
ethyl 5-chloro-6-(4-{[(2-phenylethyl)amino]carbonyl}piperazin-1-yl)nicotinate;
ethyl 6-[4-(anilinocarbonyl)piperidin-1-yl]-5-chloronicotinate;
ethyl 5-chloro-6-(4-{[(2-ethyl-6-isopropylphenyl)amino]carbonyl}piperazin-1-yl)nicotinate;
ethyl 5-cyano-6-[4-({[3-(methoxycarbonyl)phenyl]amino}carbonyl)piperazin-1-yl]-2-(trifluoromethyl)nicotinate;
ethyl 5-cyano-6-[4-({[4-(difluoromethoxy)phenyl]amino}carbonyl)piperazin-1-yl]-2-(trifluoromethyl)nicotinate;
ethyl 5-chloro-6-[4-({[3-fluoro-5-(trifluoromethyl)phenyl]amino}carbonyl)piperazin-1-yl]nicotinate;
ethyl 5-chloro-6-(4-{[(2,6-dimethoxyphenyl)amino]carbonyl}piperazin-1-yl)nicotinate; and
N-benzyl-1-[3-chloro-5-(5-ethyl-1,3-oxazol-2-yl)pyridin-2-yl]piperidine-4-carboxamide;

and pharmaceutically acceptable salts thereof.

Processes

The following processes together with the intermediates are provided as a further feature of the present invention.

Compounds of formula (I) may be prepared by the following processes a1-a5;

a1) Compounds of formula (I) in which R₁, R₂, R₃, R₄, B, R₁₄, R₁₅ and R^{d} are defined as in formula (I) above R^{c} is absent, (—NR₁₉—) or an unsubstituted, monosubstituted or polysubstituted (—N(R₁₉)—(C₁-C₄)alkylene) group, Z is an oxygen, Y is absent, X is a single bond, (—CH₂—), (—NH—CH₂—) or (—CH₂—)ₙ n=2-6 can be formed by reacting a compound of formula (II), in which R₁, R₂, R₃, R₄, B, R₁₄, and R₁₅ are defined 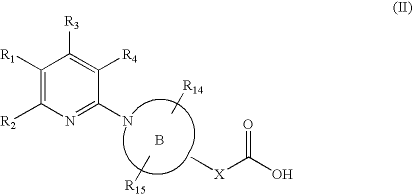
as in formula (I) above, X is a single bond, (—CH₂—), (—NH—CH₂—) or (—CH₂—)ₙ n=2-6, with a compound of formula (III) in which R^{c} is absent or an unsubstituted, monosubstituted or polysubstituted (C₁-C₄)alkylene group and R^{d} and R¹⁹ are as defined as above.
R¹⁹NH—R^{c}—R^{d} (III)
The reaction is generally carried out in an inert organic solvent such as dichloromethane at ambient temperature. The reaction may be carried out using standard conditions or in the presence of TBTU, EDCI or the combination of EDCI and HOBT. Optionally, the reaction may be carried out in the presence of an organic base such as triethylamine or DIPEA.

a2) Compounds of formula (I) in which R₁, R₂, R₃, R₄, B, R₁₄, R₁₅ and R^{d} are defined as in formula (I) above R^{c} is absent, (—NR₁₉—) or an unsubstituted, monosubstituted or polysubstituted (—N(R₁₉)—(C₁-C₄)alkylene) group, Y is absent, Z is oxygen X is a nitrogen, (CH₂—NH—) or a single bond connected to a nitrogen which is a member of the B ring, can be formed by reacting a compound of formula (IV), in which R₁, R₂, R₃, R₄, R₁₄, and R₁₅ are defined as in formula (I) above and X is a nitrogen, (—CH₂—NH₂) or a hydrogen connected to a nitrogen which is a member of the B-ring, with a compound of the general 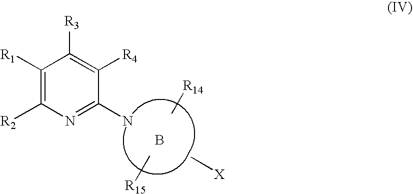
formula (III) which is defined as above.

The reaction is generally carried out in an inert solvent such as DCM. The reaction may be carried out in the presence of CDI. Optionally, the reaction may be carried out in the presence of an organic base such as triethylamine or DIPEA.

a3) Compounds of formula (I) in which R₁, R₂, R₃, R₄, B, R₁₄, R₁₅, Z, and R^{d} are defined as in formula (I) above Y is (—NH—), R^{c} is absent, an unsubstituted, monosubstituted or polysubstituted (C₁-C₄)alkylene group, (C₁-C₄)oxoalkylene group, (C₁-C₄)alkylenoxy group or oxy-(C₁-C₄)alkylene group, X is a nitrogen, (—CH₂—NH—) or a single bond connected to a nitrogen which is a member of the B ring, can be formed by reacting a compound of formula (IV) which is defined in a2) above, with a compound of formula (V)
Z=C═N—R^{c}R^{d} (V)
in which R^{c} is absent, an unsubstituted, monosubstituted or polysubstituted (C₁-C₄)alkylene group, (C₁-C₄)oxoalkylene group, (C₁-C₄)alkylenoxy group or oxy-(C₁-C₄)alkylene group and Z and R^{d} are defined as in formula (I) above.

The reaction is generally carried out in an inert solvent such as THF. Optionally, the reaction may be carried out in the presence of an organic base such as triethylamine or DIPEA.

a4) Compounds of formula (I) may also be prepared by reacting a compound of formula (VI) in which R₁, R₂, R₃ and R₄ are defined as in formula (I) above and L is a suitable leaving group, such as chloro, bromo, iodo, fluoro, triflate or tosyl, 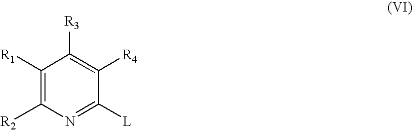
with a compound of the general formula (VII) in which X, Y, Z, B, R₁₄, R₁₅, R^{c} and R^{d} are defined as in formula (I) above. 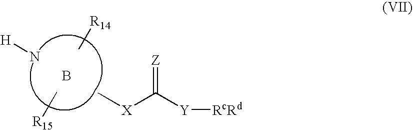
The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

a5) Compounds of formula (I) where R₁ represents R₆OC(O) and R₂, R₃, R₄, B, R₁₄, R₁₅, X, Y, Z, R^{c} and R^{d} are defined as in formula (I) above, can be transesterified using standard procedures or by reacting with R_{6′}—O⁻Li⁺ reagent, to become another compound of the general formula (I) wherein R₁ becomes R_{6′}OC(O).

The intermediates referred to above may be prepared by, for example, the methods/processes outlined below.

b) The compounds of formula (II) in which R₁, R₂, R₃, R₄, B, R₁₄, and R₁₅ are defined as in formula (I) above, X is a single bond, (—CH₂—), (—NH—CH₂—) or (—CH₂—)ₙ n=2-6, may be prepared by reacting a compound of formula (VI) defined as above, with a compound of the general formula (VIII), 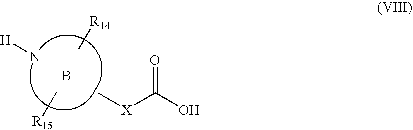
in which B, R₁₄, R₁₅ are defined as in formula (I) above and X is a single bond, (—CH₂—), (—NH—CH₂—) or (—CH₂—)ₙ n=2-6.

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

c) Compounds of formula (IV) which are defined as above may be prepared by reacting the corresponding compound of formula (VI) which is defined above, with a compound of formula (IX) in which B, R₁₄, R₁₅ are defined as in formula (I) above, X is a nitrogen, (—CH₂—NH—) or a single bond connected to a nitrogen which is a member of the B ring.

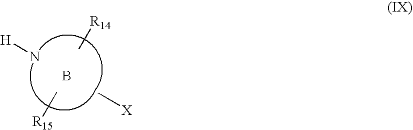

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

d) Synthesis of compounds of the general formula (XI), 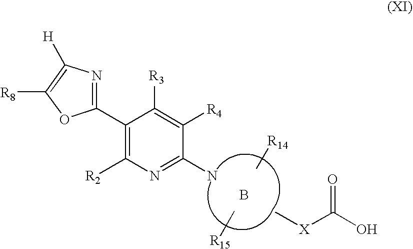
in which R₂, R₃, R₄, B, R₈, R₁₄ and R₁₅ are defined as in formula (I) above and X is a single bond, (—CH₂—), (—NH—CH₂—) or (—CH₂—)ₙ n=2-6, comprises the below steps. (d1-d₅)

d1) Reacting the corresponding compounds of the general formula (VIII) which is defined as above with a compound of the general formula (XII) 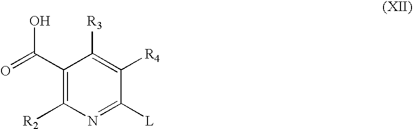
in which R₂, R₃ and R₄ are defined as in formula (I) above, and L is a suitable leaving group, such as chloro, bromo, iodo, triflate or tosyl, to give a compound of formula (XIII).

The reactions are carried out at elevated temperatures using standard equipment or a single-node microwave oven. Optionally the reaction may be carried out in the presence of an organic base such as TEA or DIPEA.

d2) The compounds of formula (XXIII) can then be reacted 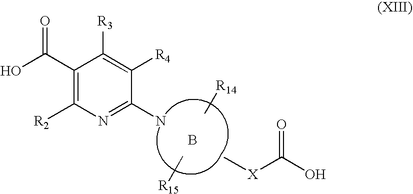
with a compound of the general formula (XIV), 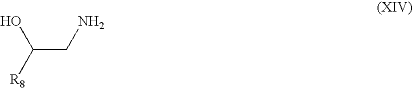
in which R₈ is defined as in formula (I) above, to give compounds of the general formula (XV). The reactions are carried out using standard conditions or in the presence of EDCI or the combination of EDCI and HOBT. Optionally the reaction may be carried out in the presence of an organic base such as TEA or DIPEA.

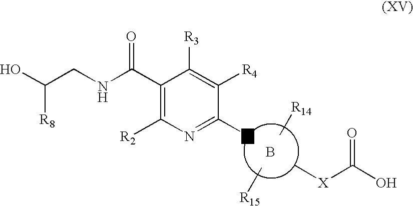

d3) This compound (XV) can then be transformed to a compound of the general formula (XVI)

d4) The preparation of compounds with the general formula (XVI), 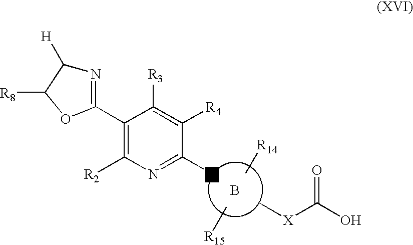
in which R₂, R₃, R₄, B, R₈, R₁₄ and R₁₅ are defined as in formula (I) above and X is a single bond, (—CH₂—), (—NH—CH₂—) or (—CH₂-)n n=2-6, using known methods or a known reagent such as methanesulfonyl chloride. Optionally the reaction may be carried out in the presence of an organic base such as TEA.

d5) a compound of the general Formula (XI) can be made by oxidizing the corresponding compound of the general formula (XVI) wherein, using a known oxidation reagent such as DDQ.

e) The preparation of compounds of the general formula (XI) also comprises the steps (e1-e4) below;

e1) Reacting a compound the general formula (XVII), 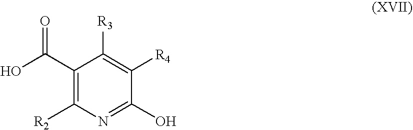
in which R₂, R₃ and R₄ are defined as in formula (I) above, with a compound of the general formula (XIX), in which R₈ is defined as in formula (I) above, 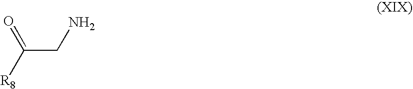
using standard conditions or in the presence of EDCI or the combination of EDCI and HOBT. Optionally the reaction may be carried out in the presence of an organic base such as TEA. This reaction gives a compound of the general formula (XX).

e2) The compound of the general formula (XX) obtained 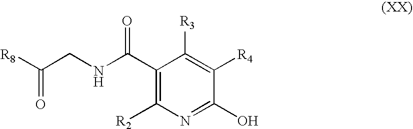
can then be transformed to a compound of the general formula (XXI), in which R₂, R₃, R₄ and R₈ are defined as in formula (I) above, using known techniques or using a known reagent such as POCl₃.

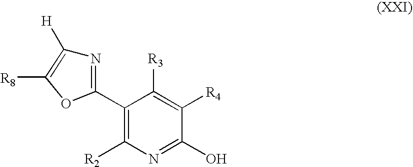

e3) A compound of the general formula (XXI) can then be transformed to a compound of the general formula (XXII), 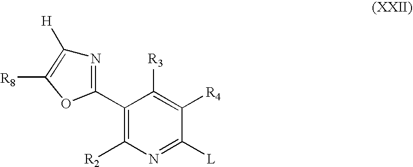
in which R₂, R₃, R₄, R₈ are defined as in formula (I) above and L is a sufficient leaving group, such as chloro, bromo, iodo, triflate or tosyl, using a known techniques or a reagent such as oxalyl chloride or thionyl chloride.

e4) The compound of formula (XXII) can then be reacted with a compound of the general formula (VIII), which is defined as above, to give a compound of the general formula (XI), defined as above. The reactions are carried out at elevated temperatures using standard equipment or a single-node microwave oven. Optionally the reactions may be carried out in the presence of an organic base such as TEA or DIPEA.

f) Preparation of compounds of the general formula (XXIII), 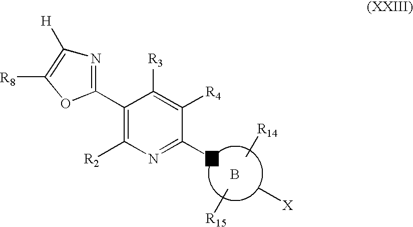
in which R₂, R₃, R₄, B, R₈, R₁₄ and R₁₅ are defined as in formula (I) above, X is a nitrogen, (—CH₂—NH—) or a single bond connected to a nitrogen which is a member of the B ring, comprises the below steps. (f1-f4)

f1) Reacting a compound of the general formula (IX) which is defined as above with a compound of the general formula (XII) which is defined as above, to give a compound of the general formula (XXIV). 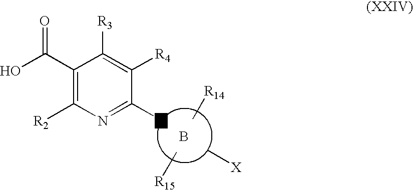
in which R₂, R₃, R₄, B, R₁₄ and R₁₅ are as defined in formula (I) above, X is a nitrogen, (—CH₂—NH—) or a single bond connected to a nitrogen which is a member of the B-ring. The reactions are carried out at elevated temperatures using standard equipment or a single-node microwave oven. Optionally the reaction may be carried out in the presence of an organic base such as TEA or DIPEA.

f2) The compound of formula (XXIV) can be reacted with a compound of formula (XIV), which is defined as above, to give compounds of the general formula (XXV). The reactions are carried out using standard conditions or in the presence of EDCI or the combination of EDCI and HOBT. Optionally the reactions may be carried out in the presence of an organic base such as TEA or DIPEA.

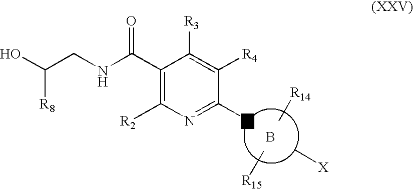

f3) This compound can then be transformed to a compound of the general formula (XXVI) in which R₂, R₃, R₄, B, R₈, R₁₄ and R₁₅, are defined as in formula (I) above, 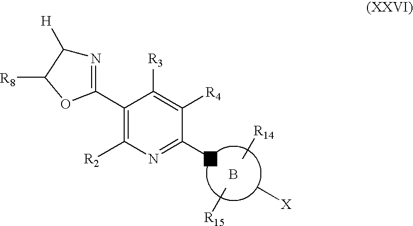
X is a nitrogen (—CH₂—NH—) or a hydrogen connected to a nitrogen which is a member of the B ring, using known methods or a sufficient reagent such as methanesulfonyl chloride. Optionally the reaction may be carried out in the presence of an organic base such as TEA.

f4) (XXIII) can then prepared by oxidising a compound of the general formula (XXVI), which is defined as above. The reaction can be performed using standard conditions or a reagent like DDQ.

Compounds of the general formula (II), in which R₁ is R₇C(O) and R₂, R₃, R₄, R₇, B, R₁₄ and R₁₅ are defined as in formula (I) above, X is a single bond comprises the following steps (g1-g2):

g1) Reacting a compound of the general formula (XIII), described above, with N,O-dimethylhydroxylamine. The reaction can be performed using known reagents like CDI to give a compound of the general formula (XXVII).

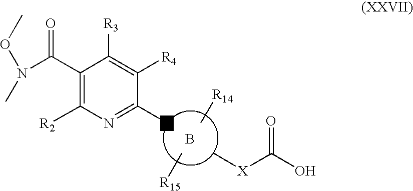

g2) Reacting compounds of the general formula (XXVII), defined as above, with a reagent of the general formula R₇—MgX′, in which R₇ is defined as in formula (I) above and X′ is a halogen, or a reagent of the formula R₇-M, in which M is a metal exemplified by Zn and Li.

Compounds of the general formula (IV), in which R₁ is R₇C(O) and R₂, R₃, R₄, R₇, B, R₁₄ and R₁₅ are defined as in formula (I) above, X is a nitrogen (—CH₂—NH—) or a single bond connected to a nitrogen which is a member of the B ring, comprises the following steps (h1-h2).

h1) Reacting a compound of the general formula (XXIV), defined as above, with N,O-dimethylhydroxylamine. The reaction can be performed using known reagents like CDI to give a compound of the general formula (XXVIII).

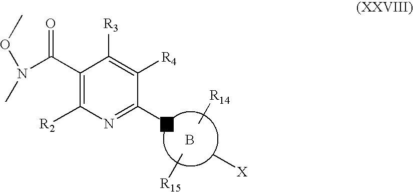

h2) A compound of the general formula (XXVIII), which is defined as above can be reacted with a reagent of the general formula R₇—MgX′, in which R₇ is defined as in formula (I) above and X′ is a halogen, or a reagent of the formula R₇-M, in which M is a metal exemplified by Zn and Li.

Compounds of the general formula (VII) can be formed in one of the processes (i1-i4). A ring nitrogen of compounds of formula (VIII) and (IX) used in the below steps may be protected by a protective group such as t-butyloxycarbonyl.

i1) Compounds of the general formula (VII) in which B, R₁₄, R₁₅ and R^{d} are defined as in formula (I) above R^{c} is absent, (—NR₁₉—) or an unsubstituted, monosubstituted or polysubstituted (—N(R₁₉)—(C₁-C₄)alkylene) group, Y is absent, Z is oxygen, X is a single bond, (—CH₂—), (—NH—CH₂—) or (—CH₂—)ₙ n=2-6, may be formed by reacting a compound of formula (VIII) with a compound of formula (III). The reaction is generally carried out in an inert organic solvent such as dichloromethane at ambient temperature. The reaction may be carried out using standard conditions or in the presence of EDCI or the combination of EDCI and HOBT. Optionally, the reaction may be carried out in the presence of an organic base such as triethylamine or DIPEA.

i2) Compounds of the general formula (VII) in which B, R₁₄, R₁₅, Z and R^{d} are defined as in formula (I) above Y is (—NH—), R^{c} is absent, an unsubstituted, monosubstituted or polysubstituted (C₁-C₄)alkylene group, (C₁-C₄)oxoalkylene group, (C₁-C₄)alkylenoxy group or oxy-(C₁-C₄)alkylene group, X is a nitrogen, (—CH₂—NH—) or a single bond connected to a nitrogen which is a member of the B ring, can be formed by reacting a compound of formula (IX) defined as above with a compound of formula (V), defined as above. The reaction is generally carried out in an inert solvent such as THF. The reaction may also be carried out in the presence of an organic base such as triethylamine or DIPEA.

i3) Compounds of the general formula (VII) in which B, R₁₄, R₁₅ and R^{d} are defined as in formula (I) above R^{c} is absent, (—NR₁₉—) or an unsubstituted, monosubstituted or polysubstituted (—N(R₁₉)—(C₁-C₄)alkylene) group, Y is absent, Z is oxygen, X is a nitrogen, (—CH₂—NH—) or a single bond connected to a nitrogen which is a member of the B ring, can also be formed by reacting a compound of formula (IX) with a compound of formula (III) which is defined as above. The reaction is generally carried out in an inert solvent such as DCM. This reaction may be carried out in the presence of CDI or a similar “—CO—” equivalent. Optionally the reaction may be carried out in the presence of an organic base such as triethylamine or DIPEA.

i4) A compound of formula (VII) which is protected with t-butoxy carbonyl may be transformed into a compound without the protective group using standard procedures or a reagent such as HCl or TFA.

(j) Compounds of the general formula (VI) which are defined as above can be formed by reacting a compound of formula (XXIX) using standard conditions or with a chlorinating reagent such as thionyl chloride or POCl₃. The reaction may be performed in an inert solvent. 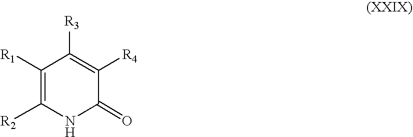
The preparation of compounds of the general formula (XXI) which is defined as above comprises the steps (k1-k3) below;

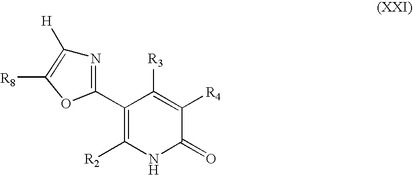

k1) Reacting a compound of the general formula (XVII) 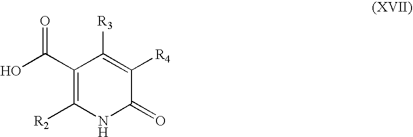
with a compound of the general formula (XIV). The reaction is generally carried out in DCM at ambient temperature. The reaction may be carried out using standard conditions or in the presence of EDCI or the combination of EDCI and HOBT. Optionally the reaction may be carried out in the presence of an organic base such as TEA or DIPEA.

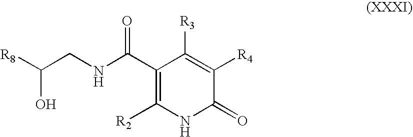

k2) The compound of formula (XXXI) can be transformed to a compound (XX) using standard conditions or an oxidising agent such as the mixture of oxalylchloride and DMSO.

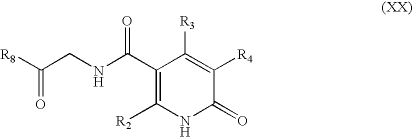

k3) The compound of formula (XX) can then be transformed into a compound of the general formula (XXI), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

l) Preparation of compounds of the general formula (XVII) which is defined as above except for R₃ which is hydrogen, comprises the following steps (l1-l3);

l1) Reacting a compound of the formula (XXXII), in which R₂ and R₆ are defined as for formula (I) with dimethoxy-N,N-dimethylmethaneamine to form a 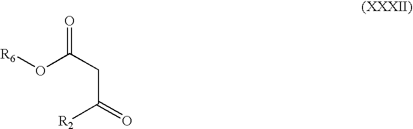
compound of formula (XXXIII).

l2) This compound (XXXIII) can then be reacted further with a compound of the 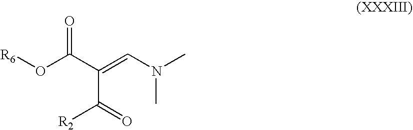
general formula R₄CH₂C(O)NH₂, in which R₄ is defined as in formula (I) above to give a compound of the general formula (XXXIV). The reaction is generally performed in an inert solvent such as ethanol, optionally in the presence of a strong base such as sodium ethoxide.

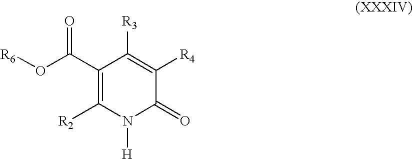

(l3) A compound of the general formula (XXXIV) can then be transformed to a compound of the general formula (XVII) defined as above except that R³ is hydrogen. The reaction is generally performed in a protic solvent such as water together with a co-solvent such as THF or methanol. The reaction can be performed using standard reagents or in the presence of LiOH, NaOH or KOH.

(m) The formation of a compound of the general formula (XI), which is defined as above can be made the below synthesis;

m1) A compound of the general formula (XXXV) where R₈ is defined as in formula (I) above can be 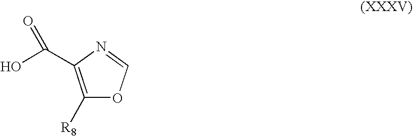
transformed in to a compound of the formula (XXXVI) 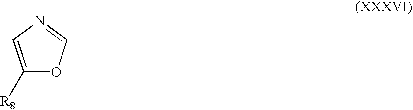
using standard conditions or using Cu(II)O and quinoline.

m2) The compound of the general formula (XXXVI) can be reacted with a compound of the general formula (XXXVII) in 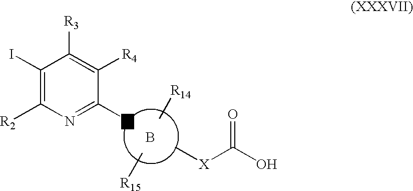
which R₂, R₃, R₄, B, R₁₄ and R₁₅ are defined as in formula (I) above and X is a single bond, (—CH₂—), (—NH—CH₂—) or (—CH₂-)n n=2-6, to give compounds of the general formula (XI). The reaction is generally performed in an inert solvent such as THF under inert atmosphere. The reaction can be performed using standard conditions or in the presence of AlkylLi such as BuLi followed by treatment with ZnCl₂ and Pd(PPh₃)₄ (preferably a catalytic amount).

(n) Compounds of the general formula (XXIII) can also be made by the step below;

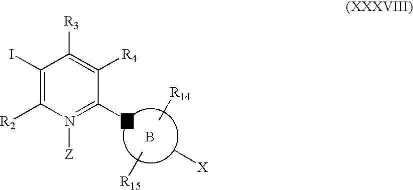

n1) Reacting a compound of the general formula (XXXVI), which is defined as above, with a compound of the general formula (XXXVIII), in which R₂, R₃, R₄, B, R₁₄ and R₁₅ are defined as in formula (I) above, X is a nitrogen, (—CH₂—NH—) or a single bond connected to a nitrogen which is a member of the B ring. The reaction is generally performed in an inert solvent such as THF under inert atmosphere. The reaction can be performed using standard conditions or in the presence of AlkylLi such as BuLi followed by treatment with ZnCl₂ and Pd(PPh₃)₄ (preferably a catalytic amount).

The preparation of compounds of the formula (III) comprises the below process. (p1)

p1) A compound of the formula LR^{c}R^{d} wherein L is a suitable leaving group, such as chloro, bromo, iodo could be transformed to the corresponding compound (III) using H₂NR¹⁹ in an inert solvent such as DMA, THF or CH₃CN. Optionally the reaction may be carried out in the presence of an organic base such as triethylamine, DIPEA or potassium carbonate.

At any stage in the synthesis of amine substituted pyridines, a chlorine substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone, thioamide or thiourea could be made from the corresponding ketone, amide and urea respectively, using known techniques or using Lawessons reagent.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscience (1999). Protected derivatives of the invention may be converted chemically to compounds of the invention using standard deprotection techniques (e.g. under alkaline or acidic conditions). The skilled person will also appreciate that certain compounds of Formula (II)-(XXXVIII) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diasteromeric derivatives by conventionals means (e.g. HPLC, chromatography over silica or crystallization). Stereocenters may also be introduced by asymmetric synthesis, (e.g metalloorganic reactions using chiral ligands). All stereoisomers are included within the scope of the invention.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

Pharmacological Data

Functional inhibition of- the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintillant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation
y=A+((B−A)/(1+((C/x)ˆD)))
and IC₅₀ estimated where:

A is the bottom plateau of the curve i.e. the final minimum y value

B is the top of the plateau of the curve i.e. the final maximum y value

C is the x value at the middle of the curve. This represents the log EC₅₀ value when A+B=100.

D is the slope factor.

x is the original known x values.

Y is the original known y values.

Most of the compounds of the invention have an activity, when tested in the functional inhibition of 2-Me-S-ADP induced P2Y₁₂ signalling assay described, at a concentration of around 3 μM or below.

For example the compounds described in Examples 14 and 63 gave the following test result in the functional inhibition of 2-Me-S-ADP induced P2Y₁₂ signalling assay described.

| | IC₅₀(μM) |
|---|---|
| Example 14 | 0.39 |
| Example 63 | 0.28 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system.

1H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Coupling constants are given in Hz.

Chromatography was performed using Biotage silica gel 40S, 40M, 121 or Merck silica gel 60 (0.063-0.200 mm). Flash chromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil C8, 10 μm columns. Reactions performed in a microwave reactor were performed in a Personal Chemistry Smith Creator, Smith synthesizer or an Emrys Optimizer.

List of Used Abbreviations:

| Abbreviation | Explanation |
|---|---|
| AcOH | Acetic acid |
| Aq | Aqueous |
| br | Broad |
| Brine | A saturated solution of sodium chloride in water |
| BSA | Bovine Serum Albumine |
| CDI | Carbonyldiimidazole |
| d | Doublet |
| DCE | 1,2-Dichloroethane |
| DCM | Dichloromethane |
| DDQ | 2,3-Dichloro-5,6-dicyano-1,4-benzoquinone |
| DIEA | N,N-Diisopropylethylamine |
| DIPEA | N,N-Diisopropylethylamine |
| DMA | N,N-Dimethylacetamide |
| DMAP | N,N-dimethylpyridin-4-amine |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| HATU | O-(7-Azabenzotriazol-1-yl)-1,1,3,3- |
| | tetramethyluromium hexafluorophosphate |
| HEPES | [4-(2-hydroxyethyl)-1-piperazineethanesulfonic acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| J | Coupling constant |
| LDA | Litiumdiisopropyl amide |
| M | Multiplet |
| MeOH | Methanol |
| MHz | Megahertz |
| mL | Millilitre |
| MS | Mass spectra |
| NBS | 1-Bromopyrrolidine-2,5-dione(N-bromo |
| | succinimide) |
| q | Quartet |
| r.t | Room temperature |
| s | Singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| TFA | Trifluoroacetic acid |
| THF | Tetrahydrofurane |
| PS-TRIS | Polymer supported Trisamine |
| TMEDA | N,N,N′,N′ Tetramethylethylenediamine |

Method A Exemplified by the Procedure from Example 56

### Ethyl 5-cyano-6-{4-[(2-naphthylamino)carbonyl]piperazin-1-yl}-2-(trifluoromethyl)nicotinate

2-isocyanatonaphthalene (20 mg, 0.12 mmol) was placed in a glass vial and a 0.14 M stock solution of ethyl 5-cyano-6-piperazin-1-yl-2-(trifluoromethyl)nicotinate (0.7 ml, 0.1 mmol) in THF was added. The reaction mixture was stirred in room temperature overnight followed by purification by HPLC (95% 0.1M ammonium acetate buffer: 5% CH₃CN→100% CH₃CN) to give ethyl 5-cyano-6-{4-[(2-naphthylamino)carbonyl]piperazin-1-yl}-2-(trifluoromethyl)nicotinate. Yield=38 mg (75%).

### Example 1

### Ethyl 6-(4-{[(4-chlorophenyl)amino]carbonyl}piperazin-1-yl)-5-cyano-2-(trifluoromethyl)nicotinate

Purchased from Maybridge Chemical Company, Cornwall UK.

### Example 2

### Ethyl 6-[4-(anilinocarbonyl)piperazin-1-yl]-5-chloronicotinate

### (a) Ethyl 5-chloro-6-piperazin-1-ylnicotinate

Ethyl 5,6-dichloronicotinate (2.20 g, 10.0 mol) was weighed into an Erlenmeyer flask. Piperazine (1.03 g, 12.0 mol), triethylamine (1.21 g, 12.0 mol), and absolute ethanol (20.0 mL) were added. The mixture was stirred until a clear solution appeared. This solution was divided into 10 microwave vials. Each vial was heated in the microwave reactor, at 120° C. for 10 minutes. The combined reaction mixtures were extracted with ethylacetate (3×80 mL) from a 10% potassium carbonate solution (80 mL). The combined organic extracts were evaporated in vacuo. The crude material was purified by flash chromatography (DCM/MeOH/triethylamine 9:1:0.1) to give Ethyl 5-chloro-6-piperazin-1-ylnicotinate. Yield: 1.60 g (61%).

¹H NMR (400 MHz, CDCl₃): 1.38 (3H, t, J=7.2 Hz), 1.77 (1H, br s), 3.01-3.05 (4H, m), 3.51-3.55 (4H, m), 4.36 (2H, t, J=7.2 Hz), 8.12 (1H, d, J=2.0 Hz), 8.75 (1H, d, J=2.0 Hz).

### (b) Ethyl 6-[4-(anilinocarbonyl)piperazin-1-yl]-5-chloronicotinate

To ethyl 5-chloro-6-piperazin-1-ylnicotinate (65 mg, 0.12 mmol) was dissolved in acetonitrile (4 mL) under a nitrogen atmosphere followed by addition of isocyanatobenzene (17 mg, 0.14 mmol). The mixture was stirred at r.t. for 22 h. PS-TRIS, ca. 100 mg, loading 4.1 mmol/g, was added and the reaction mixture was stirred gently for 2 h followed by filtration. The filtrate was further washed with DCM and the organics were combined. The solvents were removed in vacuo and the crude material was purified by flash chromatography on silica gel (pentane/ethyl acetate 5:1, then 3:1) to give ethyl 6-[4-(anilinocarbonyl)piperazin-1-yl]-5-chloronicotinate. Yield=43 mg (94%).

¹H NMR (300 MHz, CDCl₃): δ 1.38 (3H, t, J=7.1 Hz), 3.56-3.70 (8H, m), 4.36 (2H, q, J=7.1 Hz), 6.58 (1H, br. s), 7.00-7.08 (1H, m), 7.24-7.40 (4H, m), 8.15 (1H, d, J=2.0 Hz), 8.75 (1H, d, J=2.0 Hz)

### Example 3

### ethyl 6-[4-(anilinocarbonyl)piperazin-1-yl]-5-cyano-2-(trifluoromethyl)nicotinate

Purchased from Maybridge Chemical Company, Cornwall UK.

### Example 4

### ethyl 6-[4-(anilinocarbonyl)piperazin-1-yl]-5-bromonicotinate

a) Ethyl 5-bromo-6-chloronicotinate

5-Bromo-6-chloronicotinic acid (473 mg, 2.00 mmol) was suspended in absolute ethanol. Sulfuric acid (˜0.5 ml) was added and the mixture was heated to reflux for 5 h and then cooled to room temperature. The solvents were removed in vacuo. Dichloromethane (25 mL) and 1M NaOH (20 mL) was added to the residue. The phases were separated and the organic phase washed with 1M NaOH (20 mL), dried over MgSO4 and the solvents were removed in vacuo to give ethyl 5-bromo-6-chloronicotinate. Yield=410 mg. (78%)

¹H NMR (400 MHz, CDCl₃) δ 1.41 (3H, t, J=7.1 Hz), 4.42 (2H, q, J=7.1 Hz), 8.51 (1H, d, J=2 Hz), 8.91 (1H, d, J=2 Hz)

### b) ethyl 5-bromo-6-piperazin-1-ylnicotinate

Ethyl 5-bromo-6-chloronicotinate (265 mg 1.00 mmol) and piperazine (103 mg, 1.2 mmol) was suspended in ethanol. Triethylamine was added. The resulting mixture was stirred until complete dissolution of the starting materials, then it was heated at 120 degrees for 10 min in a single node microwave oven. After cooling to room temperature ethyl acetate (8 mL) and 10% aqueous K₂CO₃ (8 mL) was added. The phases were separated and the aqueous phase was extracted with ethyl acetate (2*8 mL). The combined organic extracts were evaporated in vacuo. The residue was submitted to flash chromatography (SiO2, CH₂Cl₂/MeOH/Et₃N 9:1:0.1) to give ethyl 5-bromo-6-piperazin-1-ylnicotinate. Yield=155 mg (66%).

¹H NMR (400 MHz, CDCl₃) δ 1.38 (3H, t, J=7.1 Hz), 2.24 (1H, br s), 3.03-3.07 (4H, m), 3.49-3.54 (4H, m), 4.36 (2H, d, J=7.1 Hz), 8.32 (1H, d, J=2.0 Hz), 8.79 (1H, d, J=2.0 Hz)

### c) ethyl 6-[4-(anilinocarbonyl)piperazin-1-yl]-5-bromonicotinate

A solution of isocyanatobenzene (78 mg, 0.66 mmol) acetonitrile (5 mL) was added to a solution of ethyl 5-bromo-6-piperazin-1-ylnicotinate (172 mg, 0.55 mmol) in acetonitrile (5 mL) at room temperature under nitrogen. The resulting mixture was stirred for 16 h: PS-TRIS (500 mg, 4.1 mmol/g) was added and the mixture was stirred for 2 hours. The reaction mixture was filtered and the filtrate washed with DCM. The organics were combined and the solvents were removed in vacuo to give ethyl 6-[4-(anilinocarbonyl)piperazin-1-yl]-5-bromonicotinate. Yield=226 mg (95%)

¹H NMR (400 MHz, CDCl₃) δ 1.39 (3H, t, J=7.1), 3.56-3.62 (4H, m), 3.64-3.70 (4H, m), 4.37 (2H, d, J=7.1), 6.42 (1H, br. s), 7.02-7.09 (1H, m), 7.27-7.33 (2H, m), 7.35-7.39 (2H, m), 8.36 (1H, d, J=1.8 Hz), 8.81 (1H, d, J=1.8 Hz)

### Example 5

### 3-{4-(anilinocarbonyl)-1-[3-chloro-5-(ethoxycarbonyl)pyridin-2-yl]piperazin-2-yl}propanoic acid

### a) tert-Butyl 3-[4-(anilinocarbonyl)piperazin-2-yl]propanoate

A solution of phenyl isocyanate (29 mg, 0.25 mmol) in acetonitrile (2.5 ml) was added to a solution of tert-butyl 3-piperazin-2-ylpropanoate (48 mg, 0.22 mmol) in acetonitrile (2.5 ml) under nitrogen. The resulting solution was stirred for 3 h at room temperature. PS-TRIS (200 mg, 4.1 mmol/g) was added and the suspension was stirred for 2 h. The solid material was filtered off and washed with CH₂Cl₂. The filtrate was evaporated in vacuo and the residue was submitted to flash chromatography (SiO₂, CH₂Cl₂/methanol 9:1). Yield: 40 mg (54%).

¹H NMR (400 MHz, CDCl₃): δ 1.45 (9H, s), 1.59-1.80 (2H, m), 2.34 (2H, t, J=7.5 Hz), 2.59-2.74 (2H, m), 2.82 (1H, dt, J=3.2 and 11.3 Hz), 2.92-3.08 (2H, m), 3.86-3.98 (2H, m), 6.49 (1H, s), 7.03 (1H, t, J=7.4 Hz), 7.28 (2H, t, J=7.5 Hz), 7.37 (2H, d, J=7.7 Hz). MS m/z: 334 (M+1).

### (b) Ethyl 6-[4-(anilinocarbonyl)-2-(3-tert-butoxy-3-oxopropyl)piperazin-1-yl]-5-chloronicotinate

5,6-Dichloronicotinic acid ethyl ester (26 mg, 0.12 mmol), tert-butyl 3-[4-(anilinocarbonyl)piperazin-2-yl]propanoate (37 mg, 0.11 mmol) and triethylamine (0.02 ml, 0.12 mmol) was dissolved in ethanol (1 ml). The solution was heated in a microwave reactor at 120° C. for 40 min and then at 150° C. for 20 min. A new portion of 5,6-dichloronicotinic acid ethyl ester (20 mg, 0.09 mmol) and triethylamine (0.02 ml, 0.12 mmol) was added and the solution was heated in the microwave reactor for 50 min at 120° C. The solvent was evaporated in vacuo. The residue was submitted to flash chromatography (SiO₂, heptane/ethyl acetate 3:1→2:1). Yield: 5 mg (9%).

¹H NMR (400 MHz, CDCl₃): δ 1.39 (3H, t, J=7.1 Hz), 1.50 (9H, s), 1.93-2.06 (1H, m), 2.15-2.26 (1H, m), 2.33-2.52 (2H, m), 3.06-3.24 (3H, m), 4.04-4.11 (2H, m), 4.11-4.18 (1H, m), 4.37 (2H, q, J=7.1 Hz), 4.39-4.45 (1H, m), 7.00 (1H, t, J=7.4 Hz), 7.29 (2H, t, J=8.0 Hz), 7.59 (2H, d, J=7.9 Hz), 8.15 (1H, d, J=1.8 Hz), 8.32 (1H, s br), 8.76 (1H, d, J=2.0 Hz). MS m/z: 517 (M+1).

### (c) 3-{4-(Anilinocarbonyl)-1-[3-chloro-5-(ethoxycarbonyl)pyridin-2-yl]piperazin-2-yl}propanoic acid

Ethyl 6-[4-(anilinocarbonyl)-2-(3-tert-butoxy-3-oxopropyl)piperazin-1-yl]-5-chloronicotinate (3 mg, 0.0058 mmol) was dissolved in CH₂Cl₂ (2 ml). Trifluoroacetic acid (1 ml) was added. The resulting solution was stirred at room temperature for 5 h. The solvents were removed in vacuo and the residue was coevaporated with toluene (2×3 ml). The residue was submitted to flash chromatography (SiO₂, CH₂Cl₂/methanol 15:1) to give 3-{4-(anilinocarbonyl)-1-[3-chloro-5-(ethoxycarbonyl)pyridin-2-yl]piperazin-2-yl}propanoic acid. Yield: 2 mg (75%).

¹H NMR (400 MHz, CDCl₃): δ 1.39 (3H, t, J=7.2 Hz), 2.00-2.22 (2H, m), 2.38-2.58 (2H, m), 3.01-3.17 (2H, m), 3.23 (1H, t, J=11.9 Hz), 4.04 (2H, d, J=12.7 Hz), 4.14-4.30 (2H, m), 4.37 (2H, q, J=7.1 Hz), 6.98 (1H, t, J=7.3 Hz), 7.24 (2H, t, J=7.5 Hz), 7.44 (2H, d, J=7.9 Hz), 7.79 (1H, s br), 8.15 (1H, s), 8.75 (1H, s). MS m/z: 461 (M+1).

### Example 6

### Ethyl 6-[4-(anilinocarbonyl)piperazin-1-yl]-5-cyanonicotinate

Ethyl 6-[4-(anilinocarbonyl)piperazin-1-yl]-5-bromonicotinate (43 mg, 0.100 mmol), Palladium(II) acetate (4 mg, 0.02 mmol), 1,5-bis(diphenylphosphino)pentane (18 mg, 0.04 mmol) and of TMEDA (7 drops) were mixed and toluene was added at 0° C. under a nitrogen atmosphere. The reaction mixture was stirred at r.t. for 10 minutes followed by addition of potassium cyamide (33.0 mg, 0.500 mmol). Stirred at r.t. for 30 minutes followed by stirring at 120° C. during 16 h. The reaction mixture was added 8 mL 10% sodium carbonate and was extracted with ethyl acetate (3*8 mL). The combined organic phases were dried over sodium sulphate and the solvents were removed in vacuo. The crude material was purified by flash chromatography on silica gel (pentane/ethyl acetate 2:1) to give Ethyl 6-[4-(anilinocarbonyl)piperazin-1-yl]-5-cyanonicotinate. Yield: 10 mg (26%).

¹H NMR (300 MHz, CDCl₃): δ 1.39 (3H, t, J=7.2 Hz), 3.67-3.74 (4H, m), 3.98-4.50 (4H, m), 4.37 (2H, d, J=7.2 Hz), 6.45 (1H, br s), 7.01-7.10 (1H, m), 7.24-7.4 (4H, m), 8.38 (1H, d, J=2.2 Hz), 8.90 (1H, d, J=2.2 Hz)

### Example 7

### ethyl 5-chloro-6-(4-{[(3,4-dichlorophenyl)amino]carbonyl}piperazin-1-yl)nicotinate

Prepared in according to method A from ethyl 5-chloro-6-piperazin-1-ylnicotinate and 1,2-dichloro-4-isocyanatobenzene to give Ethyl 5-chloro-6-(4-{[(3,4-dichlorophenyl)amino]carbonyl}piperazin-1-yl)nicotinate. Yield: 29.6 mg (65%).

¹H NMR (400 MHz, d₆-DMSO): δ 1.29 (3H, t, J=7.1 Hz), 3.49-3.62 (8H, m), 4.29 (2H, q, J=7.1 Hz), 7.44-7.47 (2H, m), 7.82-7.84 (1H, m), 8.10-8.12 (1H, m), 8.67-8.68 (1H, m), 8.86 (1H, s). MS m/z: 459 (M+1).

### Example 8

### ethyl 5-chloro-6-(4-{[(3,4-dichlorobenzyl)amino]carbonyl}piperazin-1-yl)nicotinate

Prepared in according to method A from ethyl 5-chloro-6-piperazin-1-ylnicotinate and 1,2-dichloro-4-(isocyanatomethyl)benzene to give Ethyl 5-chloro-6-(4-{[(3,4-dichlorobenzyl)amino]carbonyl}piperazin-1-yl)nicotinate. Yield: 32.2 mg (70%).

¹H NMR (400 MHz, d₆-DMSO): δ 1.29 (3H, t, J=7.1 Hz), 3.46 (8H, s), 4.22 (2H, d, J=5.7 Hz), 4.28 (2H, q, J=7.1 Hz), 7.19-7.26 (2H, m), 7.47-7.50 (1H, m), 7.54 (1H, d, J=8.3 Hz), 8.07-8.10 (1H, m), 8.64-8.67 (1H, m). MS m/z: 473 (M+1).

### Example 9

### ethyl 5-chloro-6-(4-{[(2-methylbenzyl)amino]carbonyl}piperazin-1-yl)nicotinate

Prepared in according to method A from ethyl 5-chloro-6-piperazin-1-ylnicotinate and 1-(isocyanatomethyl)-2-methylbenzene to give ethyl 5-chloro-6-(4-{[(2-methylbenzyl)amino]carbonyl}piperazin-1-yl)nicotinate. Yield: 26.8 mg (64%).

¹H NMR (400 MHz, d₆-DMSO): δ 1.29 (3H, t, J=7.1 Hz), 2.25 (3H, s), 3.44-3.51 (8H, m), 4.22 (2H, d, J=5.5 Hz), 4.28 (2H, q, J=7.1 Hz), 7.00 (1H, t, J=5.5 Hz), 7.08-7.15 (3H, m), 7.16-7.21 (1H, m), 8.08-8.10 (1H, m), 8.65-8.67 (1H, m). MS m/z: 418 (M+1).

### Example 10

### ethyl 5-chloro-6-(4-{[(4-fluorobenzyl)amino]carbonyl}piperazin-1-yl)nicotinate

Prepared in according to method A from ethyl 5-chloro-6-piperazin-1-ylnicotinate and 1-fluoro-4-(isocyanatomethyl)benzene to give ethyl 5-chloro-6-(4-{[(4-fluorobenzyl)amino]carbonyl}piperazin-1-yl)nicotinate. Yield=32 mg (76%)

¹H NMR (400 MHz, d₆-DMSO): δ 1.28 (3H, t, J=7.1 Hz), 3.28-3.30 (8H, m), 4.20-4.35 (2H, m), 4.28 (2H, q, J=7.1 Hz), 7.06-7.13 (2H, m), 7.14-7.19 (1H, m), 7.25-7.30 (2H, m), 8.09 (1H, d, J=2.0 Hz), 8.66 (1H, d, J=2.0 Hz)

### Example 11

### ethyl 5-chloro-6-(4-{[(3-methylbenzyl)amino]carbonyl}piperazin-1-yl)nicotinate

Prepared in according to method A from ethyl 5-chloro-6-piperazin-1-ylnicotinate and 1-(isocyanatomethyl)-3-methylbenzene to give ethyl 5-chloro-6-(4-{[(3-methylbenzyl)amino]carbonyl}piperazin-1-yl)nicotinate. Yield: 19.5 mg (47%).

¹H NMR (400 MHz, d₆-DMSO): δ 1.29 (3H, t, J=7.1 Hz), 2.26 (3H, s), 3.46 (8H, s), 4.20 (2H, d, J=5.8 Hz), 4.28 (2H, q, J=7.1 Hz), 6.97-7.07 (3H, m), 7.10 (1H, t, J=5.8 Hz), 7.16 (1H, t, J=7.5 Hz), 8.08-8.10 (1H, m), 8.65-8.67 (1H, m). MS m/z: 418 (M+1).

### Example 12

### ethyl 5-chloro-6-(4-{[(4-methylbenzyl)amino]carbonyl}piperazin-1-yl)nicotinate

Prepared in according to method A from ethyl 5-chloro-6-piperazin-1-ylnicotinate and 1-(isocyanatomethyl)-4-methylbenzene to give ethyl 5-chloro-6-(4-{[(4-methylbenzyl)amino]carbonyl}piperazin-1-yl)nicotinate. Yield: 29.2 mg (70%).

¹H NMR (400 MHz, d₆-DMSO): δ 1.29 (3H, t, J=7.1 Hz), 2.24 (3H, s), 3.45 (8H, s), 4.19 (2H, d, J=5.7 Hz), 4.28 (2H, q, J=7.1 Hz), 7.05-7.15 (5H, m), 8.07-8.10 (1H, m), 8.65-8.67 (1H, m). MS m/z: 418 (M+1).

### Example 13

### ethyl 5-chloro-6-(4-{[(3-methoxyphenyl)amino]carbonyl}piperazin-1-yl)nicotinate

Prepared in according to method A from ethyl 5-chloro-6-piperazin-1-ylnicotinate and 1-isocyanato-3-methoxybenzene to give ethyl 5-chloro-6-(4-{[(3-methoxyphenyl)amino]carbonyl}piperazin-1-yl)nicotinate. Yield: 34.7 mg (83%).

¹H NMR (400 MHz, d₆-DMSO): δ 1.29 (3H, t, J=7.1 Hz), 3.49-3.61 (8H, m), 3.69 (3H, s), 4.28 (2H, q, J=7.1 Hz), 6.48-6.52 (1H, m), 7.01-7.06 (1H, m), 7.08-7.16 (2H, m), 8.10 (1H, d, J=2.0 Hz), 8.55 (1H, s), 8.67 (1H, d, J=2.0 Hz). MS m/z: 420 (M+1).

### Example 14

### ethyl 5-chloro-6-{4-[(2-naphthylamino)carbonyl]piperazin-1-yl}nicotinate

Prepared in according to method A from ethyl 5-chloro-6-piperazin-1-ylnicotinate and 2-isocyanatonaphthalene to give ethyl 5-chloro-6-{4-[(2-naphthylamino)carbonyl]piperazin-1-yl}nicotinate. Yield: 42.4 mg (96%).

¹H NMR (400 MHz, d₆-DMSO): δ 1.29 (3H, t, J=7.1 Hz), 3.52-3.57 (4H, m), 3.62-3.67 (4H, m), 4.29 (2H, q, J=7.1 Hz), 7.29-7.35 (1H, m), 7.38-7.44 (1H, m), 7.59-7.63 (1H, m), 7.71-7.80 (3H, m), 8.00-8.03 (1H, m), 8.10-8.12 (1H, m), 8.68-8.69 (1H, m), 8.80 (1H, s). MS m/z: 440 (M+1).

### Example 15

### ethyl 6-(4-{[(3-bromophenyl)amino]carbonyl}piperazin-1-yl)-5-chloronicotinate

Prepared in according to method A from ethyl 5-chloro-6-piperazin-1-ylnicotinate and 1-bromo-3-isocyanatobenzene to give ethyl 6-(4-{[(3-bromophenyl)amino]carbonyl}piperazin-1-yl)-5-chloronicotinate. Yield: 34.6 mg (74%).

¹H NMR (400 MHz, d₆-DMSO): δ 1.29 (3H, t, J=7.1 Hz), 3.49-3.62 (8H, m), 4.28 (2H, q, J=7.1 Hz), 7.07-7.11 (1H, m), 7.18 (1H, t, J=8.1 Hz), 7.42-7.46 (1H, m), 7.77-7.79 (1H, m), 8.09-8.11 (1H, m), 8.66-8.68 (1H, m), 8.74 (1H, s). MS m/z: 469 (M+1).

### Example 16

### ethyl 5-chloro-6-[4-({[4-(methylthio)phenyl]amino}carbonyl)piperazin-1-yl]nicotinate

Prepared in according to method A from ethyl 5-chloro-6-piperazin-1-ylnicotinate and 1-isocyanato-4-(methylthio)benzene to give ethyl 5-chloro-6-[4-({[4-(methylthio)phenyl]amino}carbonyl)piperazin-1-yl]nicotinate. Yield=6.3 mg (14%).

¹H NMR (400 MHz, d₆-DMSO): δ 1.29 (3H, t, J=7.3 Hz), 2.45 (3H, s), 3.49-3.61 (8H, m), 4.29 (2H, d, J=7.1 Hz), 7.14-7.19 (2H, m), 7.40-7.45 (2H, m), 8.11 (1H, d, J=2.0 Hz), 8.59 (1H, s), 8.68 (1H, d, J=2.0 Hz)

### Example 17

### ethyl 5-chloro-6-[4-({[3-(methylthio)phenyl]amino}carbonyl)piperazin-1-yl]nicotinate

Prepared in according to method A from ethyl 5-chloro-6-piperazin-1-ylnicotinate and 1-isocyanato-3-(methylthio)benzene to give ethyl 5-chloro-6-[4-({[3-(methylthio)phenyl]amino}carbonyl)piperazin-1-yl]nicotinate. Yield: 29.1 mg (67%).

¹H NMR (400 MHz, d₆-DMSO): δ 1.29 (3H, t, J=7.1 Hz), 2.42 (3H, s), 3.49-3.55 (4H, m), 3.56-3.62 (4H, m), 4.28 (2H, q, J=7.1 Hz), 6.79-6.83 (1H, m), 7.15 (1H, t, J=7.9 Hz), 7.23-7.28 (1H, m), 7.41-7.44 (1H, m), 8.09-8.11 (1H, m), 8.59 (1H, s), 8.66-8.68 (1H, m). MS m/z: 436 (M+1).

### Example 18

### ethyl 5-chloro-6-(4-{[(3,5-dinitrophenyl)amino]carbonyl}piperazin-1-yl)nicotinate

Prepared in according to method A from ethyl 5-chloro-6-piperazin-1-ylnicotinate and 1-isocyanato-3,5-dinitrobenzene to give ethyl 5-chloro-6-(4-{[(3,5-dinitrophenyl)amino]carbonyl}piperazin-1-yl)nicotinate. Yield=39.3 mg (82%)

¹H NMR (400 MHz, d₆-DMSO): δ 1.29 (3H, t, J=7.3 Hz), 3.52-3.61 (4H, m), 3.61-3.70 (4H, m), 4.29 (2H, q, J=7.3), 7.92-7.94 (1H, m), 8.10-8.12 (1H, m), 8.36-8.38 (1H, m), 8.65-8.69 (1H, m), 8.82-8.84 (1H, m)

### Example 19

### ethyl 5-chloro-6-(4-{[(2-methoxy-5-methylphenyl)amino]carbonyl}piperazin-1-yl)nicotinate

Prepared in according to method A from ethyl 5-chloro-6-piperazin-1-ylnicotinate and 2-isocyanato-1-methoxy-4-methylbenzene to give ethyl 5-chloro-6-(4-{[(2-methoxy-5-methylphenyl)amino]carbonyl}piperazin-1-yl)nicotinate. Yield: 34.8 mg (80%).

¹H NMR (400 MHz, d₆-DMSO): δ 1.29 (3H, t, J=7.1 Hz), 2.19 (3H, s), 3.50-3.58 (8H, m), 3.75 (3H, s), 4.29 (2H, q, J=7.1 Hz), 6.77-6.81 (1H, m), 6.84-6.88 (1H, m), 7.47-7.49 (1H, m), 7.65 (1H, s), 8.09-8.12 (1H, m), 8.66-8.68 (1H, m). MS m/z: 434 (M+1).

### Example 20

### ethyl 5-chloro-6-(4-{[(3-methylphenyl)amino]carbonyl}piperazin-1-yl)nicotinate

Prepared in according to method A from ethyl 5-chloro-6-piperazin-1-ylnicotinate and 1-isocyanato-3-methylbenzene to give ethyl 5-chloro-6-(4-{[(3-methylphenyl)amino]carbonyl}piperazin-1-yl)nicotinate. Yield: 40 mg (99%).

¹H NMR (400 MHz, d₆-DMSO): δ 1.29 (3H, t, J=7.1 Hz), 2.23 (3H, s), 3.48-3.60 (8H, m), 4.29 (2H, q, J=7.1 Hz), 6.72-6.76 (1H, m), 7.09 (1H, t, J=7.8 Hz), 7.21-7.29 (2H, m), 8.10-8.12 (1H, m), 8.49 (1H, s), 8.67-8.69 (1H, m). MS m/z: 404 (M+1).

### Example 21

### ethyl 5-chloro-6-(4-{[(4-chlorophenyl)amino]carbonyl}piperazin-1-yl)nicotinate

Prepared in according to method A from ethyl 5-chloro-6-piperazin-1-ylnicotinate and 1-chloro-4-isocyanatobenzene to give ethyl 5-chloro-6-(4-{[(4-chlorophenyl)amino]carbonyl}piperazin-1-yl)nicotinate. Yield: 18.3 mg (43%).

¹H NMR (400 MHz, d₆-DMSO): δ 1.29 (3H, t, J=7.1 Hz), 3.50-3.62 (8H, m), 4.29 (2H, q, J=7.1 Hz), 7.24-7.29 (2H, m), 7.46-7.52 (2H, m), 8.11 (1H, d, J=2.0), 8.69 (1H, d, J=2.0), 8.70 (1H, s)

### Example 22

### ethyl 5-chloro-6-(4-{[(3,5-dichlorophenyl)amino]carbonyl}piperazin-1-yl)nicotinate

Prepared in according to method A from ethyl 5-chloro-6-piperazin-1-ylnicotinate and 1,3-dichloro-5-isocyanatobenzene to give ethyl 5-chloro-6-(4-{[(3,5-dichlorophenyl)amino]carbonyl}piperazin-1-yl)nicotinate. Yield: 31.1 mg (68%).

¹H NMR (400 MHz, d₆-DMSO): δ 1.29 (3H, t, J=7.1 Hz), 3.50-3.62 (8H, m), 4.29 (2H, q, J=7.1 Hz), 7.10-7.12 (1H, m), 7.57-7.60 (1H, m), 8.10-8.12 (1H, m), 8.67-8.69 (1H, m), 8.91 (1H, s). MS m/z: 459 (M+1).

### Example 23

### ethyl 5-chloro-6-(4-{[(2-isopropylphenyl)amino]carbonyl}piperazin-1-yl)nicotinate

Prepared in according to method A from ethyl 5-chloro-6-piperazin-1-ylnicotinate and 1-isocyanato-2-isopropylbenzene to give ethyl 5-chloro-6-(4-{[(2-isopropylphenyl)amino]carbonyl}piperazin-1-yl)nicotinate Yield: 24.4 mg (56%).

¹H NMR (400 MHz, d₆-DMSO): δ 1.11 (6H, d, J=6.7 Hz), 1.29 (3H, t, J=7.1 Hz), 3.12 (1H, septet, J=6.7 Hz), 3.49-3.60 (8H, m), 4.29 (2H, q, J=7.1), 7.06-7.18 (3H, m), 7.24-7.28 (1H, m), 8.10 (1H, s), 8.11 (1H, d, J=2.1 Hz), 8.68 (1H, d, J=2.1 Hz)

### Example 24

### ethyl 5-chloro-6-[4-({[(1S)-1-phenylethyl]amino}carbonyl)piperazin-1-yl]nicotinate

Prepared in according to method A from ethyl 5-chloro-6-piperazin-1-ylnicotinate and [(1S)-1-isocyanatoethyl]benzene to give ethyl 5-chloro-6-[4-({[(1S)-1-phenylethyl]amino}carbonyl)piperazin-1-yl]nicotinate. Yield: 31.9 mg (76%).

¹H NMR (400 MHz, d₆-DMSO): δ 1.29 (3H, t, J=7.1 Hz), 1.35 (3H, d, J=7.2 Hz), 3.41-3.49 (8H, m), 4.28 (2H, q, J=7.1 Hz), 4.83 (1H, p, J=7.2 Hz), 6.84 (1H, d, J=7.9 Hz), 7.14-7.20 (1H, m), 7.24-7.33 (4H, m), 8.08-8.10 (1H, m), 8.65-8.67 (1H, m). MS m/z: 418 (M+1).

### Example 25

### ethyl 5-chloro-6-[4-({[(1S)-1-(1-naphthyl)ethyl]amino}carbonyl)piperazin-1-yl]nicotinate

Prepared in according to method A from ethyl 5-chloro-6-piperazin-1-ylnicotinate and 1-[(1S)-1-isocyanatoethyl]naphthalene to give ethyl 5-chloro-6-[4-({[(1S)-1-(1-naphthyl)ethyl]amino}carbonyl)piperazin-1-yl]nicotinate. Yield: 38 mg (81%).

¹H NMR (400 MHz, d₆-DMSO): δ 1.28 (3H, t, J=7.1 Hz), 1.48 (3H, d, J=7.0 Hz), 3.40-3.52 (8H, m), 4.28 (2H, q, J=7.1 Hz), 5.65 (1H, p, 7.0 Hz), 7.02 (1H, d, J=7.6 Hz), 7.43-7.58 (4H, m), 7.77 (1H, d, J=8.1 Hz), 7.90 (1H, d, J=8.1 Hz), 8.07-8.10 (1H, m), 8.13 (1H, d, J=8.3 Hz), 8.64-8.67 (1H, m). MS m/z: 468 (M+1).

### Example 26

### ethyl 5-chloro-6-{4-[(1-naphthylamino)carbonyl]piperazin-1-yl}nicotinate

Prepared in according to method A from ethyl 5-chloro-6-piperazin-1-ylnicotinate and 1-isocyanatonaphthalene to give ethyl 5-chloro-6-{4-[(1-naphthylamino)carbonyl]piperazin-1-yl}nicotinate. Yield: 10 mg (22%).

¹H NMR (400 MHz, CDCl₃): δ 1.39 (3H, t, J=7.2 Hz), 3.63-3.75 (m, 8H), 4.38 (2H, q, J=7.2 Hz), 6.72 (1H, s br), 7.42-7.55 (3H, m), 7.64-7.70 (2H, m), 7.84-7.89 (2H, m), 8.17-8.20 (1H, m), 8.76-8.78 (1H, m). MS m/z: 440 (M+1).

### Example 27

### ethyl 5-chloro-6-(4-{[(4-methylphenyl)amino]carbonyl}piperazin-1-yl)nicotinate

Prepared in according to method A from ethyl 5-chloro-6-piperazin-1-ylnicotinate and 1-isocyanato-4-methylbenzene to give ethyl 5-chloro-6-(4-{[(4-methylphenyl)amino]carbonyl}piperazin-1-yl)nicotinate. Yield: 18.8 mg (46%).

¹H NMR (400 MHz, d₆-DMSO): δ 1.29 (3H, t, J=7.1 Hz), 2.21 (3H, s), 3.48-3.61 (8H, m), 4.28 (2H, q, J=7.1 Hz), 7.02 (2H, d, J=8.4 Hz), 7.32 (2H, d, J=8.4 Hz), 8.09-8.11 (1H, m), 8.47 (1H, s), 8.66-8.68 (1H, m). MS m/z: 404 (M+1).

### Example 28

### ethyl 5-chloro-6-(4-{[(2-methylphenyl)amino]carbonyl}piperazin-1-yl)nicotinate

Prepared in according to method A from ethyl 5-chloro-6-piperazin-1-ylnicotinate and 1-isocyanato-2-methylbenzene to give ethyl 5-chloro-6-(4-{[(2-methylphenyl)amino]carbonyl}piperazin-1-yl)nicotinate. Yield: 31 mg (77%).

¹H NMR (400 MHz, d₆-DMSO): δ 1.29 (3H, t, J=7.1), 2.15 (3H, s), 3.49-3.61 (8H, m), 4.29 (2H, d, J=7.1), 7.00-7.06 (1H, m), 7.08-7.13 (1H, m), 7.14-7.20 (2H, m), 8.10 (1H, s), 8.11 (1H, d, J=2.0 Hz), 8.68 (1H, d, J=2.0)

### Example 29

### ethyl 5-cyano-6-(4-{[(2,6-dimethoxyphenyl)amino]carbonyl}piperazin-1-yl)-2-(trifluoromethyl)nicotinate

### (a) Ethyl 5-cyano-6-piperazin-1-yl-2-(trifluoromethyl)nicotinate

Ethyl 6-chloro-5-cyano-2-(trifluoromethyl)nicotinate (1.00 g, 3.41 mmol) and piperazine (0.928 g, 10.77 mmol) was taken in ethanol (3 ml). Triethylamine (727 mg, 7.18 mmol) was added. The mixture was heated in a microwave reactor at 170° C. for 20 min. The mixture was diluted with dichloromethane (200 mL) and washed in succession with saturated sodium hydrogen carbonate solution and brine respectively. The organics were dried (Na₂SO₄), filtered and evaporated. Flash chromatography (CH₂Cl₂/MeOH 100:1 to 30:1) gave ethyl 5-cyano-6-piperazin-1-yl-2-(trifluoromethyl)nicotinate. Yield: 751 mg (67%).

¹H NMR (400, CD₃OD): δ 1.36 (3H, t, J=7.14 Hz), 2.93-2.99 (4H, m), 3.92-3.98 (4H, m), 4.34 (2H, q, J=7.22 Hz), 8.42 (1H, s). MS m/z: 329 (M+1).

### (b) Ethyl 5-cyano-6-(4-{[(2,6-dimethoxyphenyl)amino]carbonyl}piperazin-1-yl)-2-(trifluoromethyl)nicotinate

Can be prepared in according to method A from ethyl 5-cyano-6-piperazin-1-yl-2-(trifluoromethyl)nicotinate and 2-isocyanato-1,3-dimethoxybenzene to give ethyl 5-cyano-6-(4-{[(2,6-dimethoxyphenyl)amino]carbonyl}piperazin-1-yl)-2-(trifluoromethyl)nicotinate.

### Example 30

### ethyl 5-cyano-6-(4-{[(2-methoxy-5-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 2-isocyanato-1-methoxy-4-methylbenzene to give ethyl 5-cyano-6-(4-{[(2-methoxy-5-methylphenyl)amino]carbonyl}piperazin-1-yl)-2-(trifluoromethyl)nicotinate. Yield: 29.7 mg (60%).

¹H NMR (400 MHz, d₆-DMSO): δ 1.28 (3H, t, J=7.2 Hz), 2.19 (3H, s), 3.59-3.65 (4H, m), 3.76 (3H, s), 3.91-3.98 (4H, m), 4.28 (2H, q, J=7.2 Hz), 6.77-6.81 (1H, m), 6.86 (1H, d, J=8.3 Hz), 7.48-7.50 (1H, m), 7.64 (1H, s), 8.56 (1H, s). MS m/z: 492 (M+1).

### Example 31

### ethyl 5-cyano-6-(4-{[(2-isopropylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-2-isopropylbenzene to give ethyl 5-cyano-6-(4-{[(2-isopropylphenyl)amino]carbonyl}piperazin-1-yl)-2-(trifluoromethyl)nicotinate. Yield: 23 mg (47%).

¹H NMR (400 MHz, d₆-DMSO): δ 1.11 (6H, d, J=6.9 Hz), 1.28 (3H, t, J=7.1 Hz), 3.08-3.16 (1H, m), 3.59-3.66 (4H, m), 3.91-3.97 (4H, m), 4.28 (2H, q, J=7.1 Hz), 7.09-7.18 (3H, m), 7.24-7.28 (1H, m), 8.09 (1H, s), 8.56 (1H, s). MS m/z: 491 (M+1).

### Example 32

### ethyl 5-cyano-6-(4-{[(4-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-4-methylbenzene to give ethyl 5-cyano-6-(4-{[(4-methylphenyl)amino]carbonyl}piperazin-1-yl)-2-(trifluoromethyl)nicotinate. Yield: 8.9 mg (19%).

¹H NMR (400 MHz, d₆-DMSO): δ 1.28 (3H, t, J=7.1 Hz), 2.21 (3H, s), 3.60-3.66 (4H, m), 3.90-3.96 (4H, m), 4.28 (2H, q, J=7.1 Hz), 7.03 (2H, d, J=8.3 Hz), 7.33 (2H, d, J=8.3 Hz), 8.46 (1H, s), 8.56 (1H, s). MS m/z: 462 (M+1).

### Example 33

### ethyl 5-cyano-6-(4-{[(3-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-3-methylbenzene to give ethyl 5-cyano-6-(4-{[(3-methylphenyl)amino]carbonyl}piperazin-1-yl)-2-(trifluoromethyl)nicotinate. Yield: 30.3 mg (65%).

¹H NMR (400 MHz, d₆-DMSO): δ 1.28 (3H, t, J=7.1 Hz), 2.23 (3H, s), 3.60-3.66 (4H, m), 3.91-3.96 (4H, m), 4.28 (2H, q, J=7.1 Hz), 6.75 (1H, d, J=7.6 Hz), 7.10 (1H, t, J=7.8 Hz), 7.22-7.27 (1H, m), 7.27-7.30 (1H, m), 8.48 (1H, s), 8.55 (1H, s). MS m/z: 462 (M+1).

### Example 34

### ethyl 5-cyano-6-[4-({[(1S)-1-phenylethyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and [(1S)-1-isocyanatoethyl]benzene to give ethyl 5-cyano-6-[4-({[(1S)-1-phenylethyl]amino}carbonyl)piperazin-1-yl]-2-(trifluoromethyl)nicotinate. Yield: 27.4 mg (57%).

¹H NMR (400 MHz, d₆-DMSO): δ 1.27 (3H, t, J=7.1 Hz), 1.35 (3H, d, J=7.1 Hz), 3.48-3.55 (4H, m), 3.83-3.90 (4H, m), 4.27 (2H, q, J=7.1 Hz), 4.81-4.85 (1H, m), 6.84 (1H, d, J=7.9 Hz), 7.14-7.20 (1H, m), 7.24-7.33 (4H, m), 8.54 (1H, s). MS m/z: 476 (M+1).

### Example 35

### ethyl 5-cyano-6-(4-{[(2-ethoxyphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-ethoxy-2-isocyanatobenzene to give ethyl 5-cyano-6-(4-{[(2-ethoxyphenyl)amino]carbonyl}piperazin-1-yl)-2-(trifluoromethyl)nicotinate. Yield: 30.5 mg (62%).

¹H NMR (400 MHz, d₆-DMSO): δ 1.28 (3H, t, J=7.1), 1.29 (3H, t, J=7.1), 3.62-3.68 (4H, m), 3.93-3.99 (4H, m), 4.05 (2H, q, J=7.1), 4.28 (2H, q, J=7.1), 6.82-6.90 (1H, m), 6.94-7.00 (2H, m), 7.61-7.64 (1H, m), 7.68-7.74 (1H, m), 8.54-8.57 (1H, m)

### Example 36

### ethyl 6-(4-{[(2-chlorophenyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-chloro-2-isocyanatobenzene to give ethyl 6-(4-{[(2-chlorophenyl)amino]carbonyl}piperazin-1-yl)-5-cyano-2-(trifluoromethyl)nicotinate. Yield: 31 mg (64%).

¹H NMR (400 MHz, d₆-DMSO): δ 1.28 (3H, t, J=7.1 Hz), 3.62-3.69 (4H, m), 3.92-3.98 (4H, m), 4.28 (2H, q, J=7.1 Hz), 7.10-7.16 (1H, m), 7.25-7.30 (1H, m), 7.42-7.46 (1H, m), 7.49-7.54 (1H, m), 8.24 (1H, s), 8.56 (1H, s). MS m/z: 483 (M+1).

### Example 37

### ethyl 5-cyano-6-(4-{[(2-methylbenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-(isocyanatomethyl)-2-methylbenzene to give ethyl 5-cyano-6-(4-{[(2-methylbenzyl)amino]carbonyl}piperazin-1-yl)-2-(trifluoromethyl)nicotinate. Yield: 25.5 mg (53%).

¹H NMR (400 MHz, d₆-DMSO): δ 1.28 (3H, t, J=7.1 Hz), 2.26 (3H, s), 3.50-3.57 (4H, m), 3.85-3.91 (4H, m), 4.22 (2H, d, J=5.4 Hz), 4.27 (2H, q, J=7.1 Hz), 6.96-7.02 (1H, m), 7.08-7.15 (3H, m), 7.16-7.22 (1H, m), 8.54 (1H, s). MS m/z: 476 (M+1).

### Example 38

### ethyl 6-(4-{[(2-chlorobenzyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-chloro-2-(isocyanatomethyl)benzene to give ethyl 6-(4-{[(2-chlorobenzyl)amino]carbonyl}piperazin-1-yl)-5-cyano-2-(trifluoro-methyl)nicotinate. Yield: 31.6 mg (63%).

¹H NMR (400 MHz, d₆-DMSO): δ 1.28 (3H, t, J=7.1 Hz), 3.53-3.59 (4H, m), 3.87-3.93 (4H, m), 4.24-4.34 (4H, m), 7.13-7.19 (1H, m), 7.21-7.35 (3H, m), 7.37-7.41 (1H, m), 8.55 (1H, s). MS m/z: 497 (M+1).

### Example 39

### ethyl 5-cyano-6-(4-{[(4-fluorobenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-fluoro-4-(isocyanatomethyl)benzene to give ethyl 5-cyano-6-(4-{[(4-fluorobenzyl)amino]carbonyl}piperazin-1-yl)-2-(trifluoro-methyl)nicotinate. Yield: 28.2 mg (58%).

¹H NMR (400 MHz, d₆-DMSO): δ 1.27 (3H, t, J=7.3 Hz), 3.49-3.55 (4H, m), 3.84-3.91 (4H, m), 4.22 (2H, d, J=5.8 Hz), 4.27 (2H, q, J=7.3 Hz), 7.06-7.18 (3H, m), 7.25-7.32 (2H, m), 8.54 (1H, s). MS m/z: 480 (M+1).

### Example 40

### ethyl 5-cyano-6-[4-({[(1R,2R)-2-phenylcyclopropyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and [(1R,2R)-2-isocyanatocyclopropyl]benzene to give ethyl 5-cyano-6-[4-({[(1R,2R)-2-phenylcyclopropyl]amino}carbonyl)piperazin-1-yl]-2-(trifluoromethyl)nicotinate. Yield: 32.2 mg (66%).

¹H NMR (400 MHz, d₆-DMSO): δ 1.04-1.11 (1H, m), 1.12-1.19 (1H, m), 1.27 (3H, t, J=7.1 Hz), 1.85-1.92 (1H, m), 2.66-2.72 (1H, m), 3.44-3.50 (4H, m), 3.83-3.89 (4H, m), 4.27 (2H, q, J=7.1 Hz), 6.85-6.89 (1H, m), 7.06-7.15 (3H, m), 7.20-7.25 (2H, m), 8.54 (1H, s). MS m/z: 488 (M+1).

### Example 41

### ethyl 5-cyano-6-(4-{[(3-methylbenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-(isocyanatomethyl)-3-methylbenzene to give ethyl 5-cyano-6-(4-{[(3-methylbenzyl)amino]carbonyl}piperazin-1-yl)-2-(trifluoromethyl)nicotinate. Yield: 39.7 mg (83%).

¹H NMR (400 MHz, d₆-DMSO): δ 1.27 (3H, t, J=7.1 Hz), 2.26 (3H, s), 3.49-3.55 (4H, m), 3.85-3.90 (4H, m), 4.21 (2H, d, J=5.6 Hz), 4.27 (2H, q, J=7.1 Hz), 6.98-7.12 (4H, m), 7.16 (1H, t, J=7.5 Hz), 8.54 (1H, s). MS m/z: 476 (M+1).

### Example 42

### ethyl 5-cyano-6-(4-{[(4-methylbenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-(isocyanatomethyl)-4-methylbenzene to give ethyl 5-cyano-6-(4-{[(4-methylbenzyl)amino]carbonyl}piperazin-1-yl)-2-(trifluoromethyl)nicotinate. Yield: 23.5 mg (49%).

¹H NMR (400 MHz, d₆-DMSO): δ 1.27 (3H, t, J=7.1 Hz), 2.24 (3H, s), 3.48-3.54 (4H, m), 3.84-3.90 (4H, m), 4.20 (2H, d, J=5.7 Hz), 4.27 (2H, q, J=7.1 Hz), 7.05-7.11 (3H, m), 7.11-7.16 (2H, m), 8.53 (1H, s). MS m/z: 476 (M+1).

### Example 43

### ethyl 5-cyano-6-(4-{[(3,4-dichlorobenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1,2-dichloro-4-(isocyanatomethyl)benzene to give ethyl 5-cyano-6-(4-{[(3,4-dichlorobenzyl)amino]carbonyl}piperazin-1-yl)-2-(trifluoromethyl)nicotinate. Yield: 53 mg (99%).

¹H NMR (400 MHz, d₆-DMSO): δ 1.27 (3H, t, J=7.1 Hz), 3.49-3.55 (4H, m), 3.85-3.91 (4H, m), 4.22 (2H, d, J=5.7 Hz), 4.27 (2H, q, J=7.1 Hz), 7.18-7.27 (2H, m), 7.48-7.50 (1H, m), 7.55 (1H, d, J=8.2 Hz), 8.54 (1H, s). MS m/z: 531 (M+1).

### Example 44

### ethyl 5-cyano-6-(4-{[(3-methoxyphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-3-methoxybenzene to give ethyl 5-cyano-6-(4-{[(3-methoxyphenyl)amino]carbonyl}piperazin-1-yl)-2-(trifluoromethyl)nicotinate. Yield: 32.7 mg (68%).

¹H NMR (400 MHz, d₆-DMSO): δ 1.28 (3H, t, J=7.1 Hz), 3.61-3.66 (4H, m), 3.69 (1H, s), 3.91-3.96 (4H, m), 4.28 (2H, q, J=7.1 Hz), 6.48-6.53 (1H, m), 7.02-7.06 (1H, m), 7.08-7.17 (2H, m), 8.53 (1H, s), 8.56 (1H, s). MS m/z: 478 (M+1).

### Example 45

### ethyl 5-cyano-6-(4-{[(2-fluoro-5-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-fluoro-2-isocyanato-4-methylbenzene to give ethyl 5-cyano-6-(4-{[(2-fluoro-5-methylphenyl)amino]carbonyl}piperazin-1-yl)-2-(trifluoromethyl)nicotinate. Yield: 27.3 mg (57%).

¹H NMR (400 MHz, d₆-DMSO): δ 1.28 (3H, t, J=7.1 Hz), 2.23 (3H, s), 3.59-3.66 (4H, m), 3.90-3.97 (4H, m), 4.28 (2H, q, J=7.1 Hz), 3.86-3.92 (1H, m), 7.00-7.09 (1H, m), 7.25 (1H, d, J=7.7 Hz), 8.28 (1H, s), 8.56 (1H, s). MS m/z: 480 (M+1).

### Example 46

### ethyl 6-(4-{[(3-chlorophenyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-chloro-3-isocyanatobenzene to give ethyl 6-(4-{[(3-chlorophenyl)amino]carbonyl}piperazin-1-yl)-5-cyano-2-(trifluoromethyl)nicotinate. Yield: 30.6 mg (63%).

¹H NMR (400 MHz, d₆-DMSO): δ 1.28 (3H, t, J=7.1 Hz), 3.62-3.68 (4H, m), 3.91-3.97 (4H, m), 4.28 (2H, q, J=7.1 Hz), 6.95-6.99 (1H, m), 7.24 (1H, t, J=8.1 Hz), 7.37-7.41 (1H, m), 7.63-7.66 (1H, m), 8.56 (1H, s), 8.74 (1H, s). MS m/z: 483 (M+1).

### Example 47

### ethyl 5-cyano-6-[4-({[2-(2-thienyl)ethyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 2-(2-isocyanatoethyl)thiophene to give ethyl 5-cyano-6-[4-({[2-(2-thienyl)ethyl]amino}carbonyl)piperazin-1-yl]-2-(trifluoromethyl)nicotinate. Yield: 30.5 mg (63%).

¹H NMR (400 MHz, d₆-DMSO): δ 1.27 (3H, t, J=7.1 Hz), 2.93 (2H, t, J=7.2 Hz), 3.22-3.31 (2H, m), 3.45-3.51 (4H, m), 3.83-3.89 (4H, m), 4.27 (2H, q, J=7.1 Hz), 6.72-6.78 (1H, m), 6.83-6.87 (1H, m), 6.90-6.95 (1H, m), 7.30 (1H, d, J=5.1 Hz), 8.54 (1H, s).

MS m/z: 483 (M+1).

### Example 48

### ethyl 5-cyano-6-(4-{[(3-cyanophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 3-isocyanatobenzonitrile to give ethyl 5-cyano-6-(4-{[(3-cyanophenyl)amino]carbonyl}piperazin-1-yl)-2-(trifluoromethyl)nicotinate. Yield: 30.8 mg (65%).

¹H NMR (400 MHz, d₆-DMSO): δ 1.28 (3H, t, J=7.1 Hz), 3.63-3.70 (4H, m), 3.92-3.98 (4H, m), 4.28 (2H, q, J=7.1 Hz), 7.35-7.39 (1H, m), 7.45 (1H, t, J=8.0 Hz), 7.72-7.77 (1H, m), 7.92-7.95 (1H, m), 8.56 (1H, s), 8.90 (1H, s). MS m/z: 473 (M+1).

### Example 49

### ethyl 5-cyano-6-(4-{[(2-methoxyphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-2-methoxybenzene to give ethyl 5-cyano-6-(4-{[(2-methoxyphenyl)amino]carbonyl}piperazin-1-yl)-2-(trifluoromethyl)nicotinate. Yield: 31.4 mg (65%).

¹H NMR (400 MHz, d₆-DMSO): δ 1.28 (3H, t, J=7.1 Hz), 3.60-3.66 (4H, m), 3.80 (3H, s), 3.92-3.98 (4H, m), 4.28 (2H, q, J=7.1 Hz), 6.83-6.89 (1H, m), 6.97-7.02 (2H, m), 7.63-7.67 (1H, m), 7.70 (1H, s), 8.55 (1H, s). MS m/z: 478 (M+1).

### Example 50

### ethyl 6-{4-[(benzylamino)carbonyl]piperazin-1-yl}-5-cyano-2-(trifluoromethyl)nicotinate

Prepared in according to method A from ethyl 5-cyano-6-piperazin-1-yl-2-(trifluoromethyl)nicotinate and (isocyanatomethyl)benzene to give ethyl 6-{4-[(benzylamino)carbonyl]piperazin-1-yl}-5-cyano-2-(trifluoromethyl)nicotinate. Yield: 32.9 mg (71%).

¹H NMR (400 MHz, d₆-DMSO): δ 1.27 (3H, t, J=7.1 Hz), 3.50-3.55 (4H, m), 3.85-3.91 (4H, m), 4.23-4.31 (4H, m), 7.10-7.32 (6H, m), 8.54 (1H, s). MS m/z: 462 (M+1).

### Example 51

### ethyl 6-(4-{[(5-chloro-2,4-dimethoxyphenyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-chloro-5-isocyanato-2,4-dimethoxybenzene to give ethyl 6-(4-{[(5-chloro-2,4-dimethoxyphenyl)amino]carbonyl}piperazin-1-yl)-5-cyano-2-(trifluoromethyl)nicotinate. Yield: 32.3 mg (59%).

¹H NMR (400 MHz, d₆-DMSO): δ 1.28 (3H, t, J=7.1 Hz), 3.57-3.65 (4H, m), 3.84 (3H, s), 3.85 (3H, s), 3.90-3.97 (4H, m), 4.28 (2H, q, J=7.1 Hz), 6.80 (1H, s), 7.56 (1H, s), 7.73 (1H, s), 8.55 (1H, s). MS m/z: 543 (M+1).

### Example 52

### ethyl 5-cyano-6-(4-{[(3-nitrophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-3-nitrobenzene to give ethyl 5-cyano-6-(4-{[(3-nitrophenyl)amino]carbonyl}piperazin-1-yl)-2-(trifluoromethyl)nicotinate. Yield: 25.8 mg (52%).

¹H NMR (400 MHz, d₆-DMSO): δ 1.28 (3H, t, J=7.1 Hz), 3.65-3.71 (4H, m), 3.93-3.99 (4H, m), 4.28 (2H, q, J=7.1 Hz), 7.52 (1H, t, J=8.2 Hz), 7.75-7.80 (1H, m), 7.87-7.92 (1H, m), 8.46-8.49 (1H, m), 8.56 (1H, s), 9.07 (1H, s). MS m/z: 493 (M+1).

### Example 53

### ethyl 5-cyano-6-[4-({[3-fluoro-5-(trifluoromethyl)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 1-fluoro-3-isocyanato-5-(trifluoromethyl)benzene to give ethyl 5-cyano-6-[4-({[3-fluoro-5-(trifluoromethyl)phenyl]amino}carbonyl)piperazin-1-yl]-2-(trifluoromethyl)nicotinate. Yield: 33.3 mg (62%). MS m/z: 534 (M+1).

### Example 54

### ethyl 5-cyano-6-[4-({[3-(methylthio)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 1-isocyanato-3-(methylthio)benzene to give ethyl 5-cyano-6-[4-({[3-(methylthio)phenyl]amino}carbonyl)piperazin-1-yl]-2-(trifluoromethyl)nicotinate. Yield: 28.1 mg (57%).

¹H NMR (400 MHz, d₆-DMSO): δ 1.28 (3H, t, J=7.1 Hz), 2.42 (3H, s), 3.60-3.68 (4H, m), 3.90-3.98 (4H, m), 4.28 (2H, q, J=7.1 Hz), 6.82 (1H, d, J=7.8 Hz), 7.16 (1H, t, J=8.0 Hz), 7.26 (1H, d, J=8.1 Hz), 7.43 (1H, s), 8.56 (1H, s), 8.57 (1H, s). MS m/z: 495 (M+1).

### Example 55

### ethyl 5-cyano-6-(4-{[(3-fluorobenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-fluoro-3-(isocyanatomethyl)benzene to give ethyl 5-cyano-6-(4-{[(3-fluorobenzyl)amino]carbonyl}piperazin-1-yl)-2-(trifluoromethyl)nicotinate. Yield: 48 mg (100%).

¹H NMR (400 MHz, d₆-DMSO): δ 1.27 (3H, t, J=7.2 Hz), 3.50-3.56 (4H, m), 3.85-3.91 (4H, m), 4.23-4.31 (4H, m), 6.97-7.12 (3H, m), 7.15-7.22 (1H, m), 7.28-7.36 (1H, m), 8.54 (1H, s). MS m/z: 480 (M+1).

### Example 56

### ethyl 5-cyano-6-{4-[(2-naphthylamino)carbonyl]piperazin-1-yl}-2-(trifluoromethyl)nicotinate

Prepared in according to method A from ethyl 5-cyano-6-piperazin-1-yl-2-(trifluoromethyl)nicotinate and 2-isocyanatonaphthalene to give ethyl 5-cyano-6-{4-[(2-naphthylamino)carbonyl]piperazin-1-yl}-2-(trifluoromethyl)nicotinate. Yield=38 mg (75%).

¹H NMR (400 MHz, d₆-DMSO): δ 1.28 (3H, t, J=7.2 Hz), 3.67-3.73 (4H, m), 3.94-4.01 (4H, m), 4.29 (2H, q, J=7.2 Hz), 7.3-7.36 (1H, m), 7.41 (1H, t, J=7.3 Hz), 7.58-7.64 (1H, m), 7.73 (1H, d, J=8.3 Hz), 7.78 (2H, d, J=8.5 Hz), 8.03 (1H, s), 8.56 (1H, s), 8.78 (1H, s). MS m/z: 498 (M+1).

### Example 57

### ethyl 6-(4-{[(3-bromophenyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-bromo-3-isocyanatobenzene to give ethyl 6-(4-{[(3-bromophenyl)amino]carbonyl}piperazin-1-yl)-5-cyano-2-(trifluoromethyl)nicotinate. Yield: 35.7 mg (67%).

¹H NMR (400 MHz, d₆-DMSO): δ 1.28 (3H, t, J=7.1 Hz), 3.62-3.68 (4H, m), 3.91-3.97 (4H, m), 4.28 (2H, q, J=7.1 Hz), 7.08-7.12 (1H, m), 7.19 (1H, t, J=8.0 Hz), 7.42-7.46 (1H, m), 7.77-7.80 (1H, m), 8.56 (1H, s), 8.73 (1H, s). MS m/z: 527 (M+1).

### Example 58

### ethyl 6-(4-{[(4-bromophenyl)amino]carbonyl}piperazin-1-yl)-5-cyano-2-(trifluoromethyl)nicotinate

Prepared in according to method A from ethyl 5-cyano-6-piperazin-1-yl-2-(trifluoromethyl)nicotinate and 1-bromo-4-isocyanatobenzene to give ethyl 6-(4-{[(4-bromophenyl)amino]carbonyl}piperazin-1-yl)-5-cyano-2-(trifluoromethyl)nicotinate. Yield: 17.6 mg (33%).

¹H NMR (400 MHz, d₆-DMSO): δ 1.28 (3H, t, J=7.1 Hz), 3.61-3.67 (4H, m), 3.91-3.97 (4H, m), 4.28 (2H, q, J=7.1 Hz), 7.37-7.42 (2H, m), 7.42-7.47 (2H, m), 8.55 (1H, s), 8.68 (1H, s). MS m/z: 427 (M+1).

### Example 59

### ethyl 6-(4-{[(2-bromophenyl)amino]carbonyl}piperazin-1-yl)-5-chloronicotinate

Prepared in according to method A from 5-chloro-6-piperazin-1-ylnicotinate and 1-bromo-2-isocyanatobenzene to give ethyl 6-(4-{[(2-bromophenyl)amino]carbonyl}piperazin-1-yl)-5-chloronicotinate. Yield: 31.7 mg (67%).

¹H NMR (400 MHz, d₆-DMSO): δ 1.29 (3H, t, J=7.1), 3.50-3.64 (8H, m), 4.06 (2H, q, J=7.1), 7.04-7.10 (1H, m), 7.29-7.35 (1H, m), 7.45-7.50 (1H, m), 7.57-7.62 (1H, m), 8.11 (1H, d, J=2.0), 8.24 (1H, s), 8.68 (1H, d, J=2.0)

### Example 60

### ethyl 5-chloro-6-[4-({[1-(3-isopropenylphenyl)-1-methylethyl]amino}carbonyl)piperazin-1-yl]nicotinate

Prepared in according to method A from 5-chloro-6-piperazin-1-ylnicotinate and 1-(1-isocyanato-1-methylethyl)-3-isopropenylbenzene to give ethyl 5-chloro-6-[4-({[1-(3-isopropenylphenyl)-1-methylethyl]amino}carbonyl)piperazin-1-yl]nicotinate. Yield: 26.4 mg (56%).

¹H NMR (400 MHz, d₆-DMSO): δ 1.29 (3H, t, J=7.1 Hz), 1.55 (6H, s), 2.07 (3H, s), 3.44 (8H, s), 4.29 (2H, q, J=7.1 Hz), 5.03-5.06 (1H, m), 5.34 (1H, s), 6.59 (1H, s), 7.19-7.27 (3H, m), 7.41-7.43 (1H, m), 8.09-8.11 (1H, m), 8.66-8.68 (1H, m). MS m/z: 472 (M+1).

### Example 61

### ethyl 5-chloro-6-(4-{[(2-methyl-3-nitrophenyl)amino]carbonyl}piperazin-1-yl)nicotinate

Prepared in according to method A from 5-chloro-6-piperazin-1-ylnicotinate and 1-isocyanato-2-methyl-3-nitrobenzene to give Ethyl 5-chloro-6-(4-{[(2-methyl-3-nitrophenyl)amino]carbonyl}piperazin-1-yl)nicotinate. Yield: 7.5 mg (16%).

¹H NMR (400 MHz, d₆-DMSO): δ 1.29 (3H, t, J=7.1 Hz), 2.20 (3H, s), 3.51-3.64 (8H, m), 4.29 (2H, q, J=7.1 Hz), 7.37 (1H, t, J=8.1 Hz), 7.48-7.52 (1H, m), 7.63-7.68 (1H, m), 8.10-8.13 (1H, m), 8.56 (1H, s), 8.67-8.70 (1H, m). MS m/z: 449 (M+1).

### Example 62

### ethyl 5-chloro-6-{4-[(2-thienylamino)carbonyl]piperazin-1-yl}nicotinate

Prepared in according to method A from 5-chloro-6-piperazin-1-ylnicotinate and 2-isocyanatothiophene to give ethyl 5-chloro-6-{4-[(2-thienylamino)carbonyl]piperazin-1-yl}nicotinate. Yield: 11.1 mg (28%).

¹H-NMR (400 MHz, d₆-DMSO): δ 1.29 (3H, t, J=7.1), 3.49-3.62 (8H, m), 4.29 (2H, q, J=7.1), 6.58-6.62 (1H, m), 6.74-6.81 (2H, m), 8.10-13 (1H, m), 8.67-8.69 (1H, m)

### Example 63

### ethyl 5-chloro-6-(4-{[(3-chlorophenyl)amino]carbonyl}piperazin-1-yl)nicotinate

Prepared in according to method A from 5-chloro-6-piperazin-1-ylnicotinate and 1-isocyanato-2-methyl-3-nitrobenzene to give Ethyl 5-chloro-6-(4-{[(3-chlorophenyl)amino]carbonyl}piperazin-1-yl)nicotinate. Yield: 31.9 mg (75%).

¹H NMR (400 MHz, d₆-DMSO): δ 1.29 (3H, t, J=7.1 Hz), 3.49-3.63 (8H, m), 4.29 (2H, q, J=7.1 Hz), 6.94-6.99 (1H, m), 7.24 (1H, t, J=8.1 Hz), 7.37-7.41 (1H, m), 7.63-7.66 (1H, m), 8.10-8.12 (1H, m), 8.66-8.69 (1H, m), 8.76 (1H, s). MS m/z: 424 (M+1).

### Example 64

### ethyl 5-cyano-6-(4-{[(3,5-dichlorophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1,3-dichloro-5-isocyanatobenzene to give ethyl 5-cyano-6-(4-{[(3,5-dichlorophenyl)amino]carbonyl}piperazin-1-yl)-2-(trifluoromethyl)nicotinate. Yield: 11.8 mg (22%).

¹H NMR (400 MHz, d₆-DMSO): δ 1.28 (3H, t, J=7.1 Hz), 3.62-3.68 (4H, m), 3.91-3.97 (4H, m), 4.28 (2H, q, J=7.1 Hz), 7.10-7.12 (1H, m), 7.58-7.60 (2H, m), 8.56 (1H, s), 8.91 (1H, s). MS m/z: 517 (M+1).

### Example 65

### ethyl 5-cyano-6-(4-{[(2-methyl-3-nitrophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-2-methyl-3-nitrobenzene to give ethyl 5-cyano-6-(4-{[(2-methyl-3-nitrophenyl)amino]carbonyl}piperazin-1-yl)-2-(trifluoromethyl)nicotinate. Yield: 5.9 mg (111%).

¹H NMR (400 MHz, d₆-DMSO): δ 1.28 (3H, t, J=7.1 Hz), 2.21 (3H, s), 3.63-3.69 (4H, m), 3.92-3.99 (4H, m), 4.28 (2H, q, J=7.1 Hz), 7.37 (1H, t, J=8.1 Hz), 7.49-7.53 (1H, m), 7.63-7.68 (1H, m), 8.54 (1H, s), 8.57 (1H, s). MS m/z: 507 (M+1).

### Example 66

### ethyl 6-{4-[(biphenyl-2-ylamino)carbonyl]piperazin-1-yl}-5-cyano-2-(trifluoromethyl)nicotinate

Prepared in according to method A from ethyl 5-cyano-6-piperazin-1-yl-2-(trifluoromethyl)nicotinate and 2-isocyanatobiphenyl to give ethyl 6-{4-[(biphenyl-2-ylamino)carbonyl]piperazin-1-yl}-5-cyano-2-(trifluoromethyl)nicotinate. Yield: 24.4 mg (46%).

¹H NMR (400 MHz, d₆-DMSO): δ 1.28 (3H, t, J=7.1 Hz), 3.43-3.49 (4H, m), 3.76-3.81 (4H, m), 4.28 (2H, q, J=7.1 Hz), 7.20-7.40 (9H, m), 8.04 (1H, s), 8.54 (1H, s).

MS m/z: 525 (M+1).

### Example 67

### ethyl 5-cyano-6-(4-{[(3,4-dichlorophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1,2-dichloro-4-isocyanatobenzene to give ethyl 5-cyano-6-(4-{[(3,4-dichlorophenyl)amino]carbonyl}piperazin-1-yl)-2-(trifluoromethyl)nicotinate. Yield: 27.2 mg (52%).

¹H NMR (400 MHz, d₆-DMSO): δ 1.28 (3H, t, J=7.1 Hz), 3.61-3.69 (4H, m), 3.91-3.98 (4H, m), 4.27 (2H, q, J=7.1 Hz), 7.44-7.47 (2H, m), 7.82-7.85 (1H, m), 8.55 (1H, s), 8.84 (1H, s). MS m/z: 517 (M+1).

### Example 68

### ethyl 5-cyano-6-[4-({[1-(3-isopropenylphenyl)-1-methylethyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 1-(1-isocyanato-1-methylethyl)-3-isopropenylbenzene to give ethyl 5-cyano-6-[4-({[1-(3-isopropenylphenyl)-1-methylethyl]amino}carbonyl)piperazin-1-yl]-2-(trifluoromethyl)nicotinate. Yield: 9.3 mg (17%).

¹H NMR (400 MHz, d₆-DMSO): δ 1.28 (3H, t, J=7.1 Hz), 1.56 (6H, s), 2.07 (3H, s), 3.45-3.52 (4H, m), 3.82-3.89 (4H, m), 4.27 (2H, q, J=7.1 Hz), 5.04 (1H, s), 5.33 (1H, s), 6.59 (1H, s), 7.18-7.27 (3H, m), 7.42 (1H, s), 8.55 (1H, s). MS m/z: 531 (M+1).

### Example 69

### ethyl 5-cyano-6-(4-{[(4-phenoxyphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-4-phenoxybenzene to give ethyl 5-cyano-6-(4-{[(4-phenoxyphenyl)amino]carbonyl}piperazin-1-yl)-2-(trifluoromethyl)nicotinate. Yield: 31.9 mg (59%).

¹H NMR (400 MHz, d₆-DMSO): δ 1.28 (3H, t, J=7.1 Hz), 3.61-3.68 (4H, m), 3.91-3.98 (4H, m), 4.28 (2H, q, J=7.1 Hz), 6.90-6.96 (4H, m), 7.03-7.09 (1H, m), 7.30-7.36 (2H, m), 7.43-7.49 (2H, m), 8.56 (1H, s), 8.58 (1H, s). MS m/z: 541 (M+1).

### Example 70

### ethyl 5-cyano-6-(4-{[(4-methoxybenzyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-(isocyanatomethyl)-4-methoxybenzene to give ethyl 5-cyano-6-(4-{[(4-methoxybenzyl)amino]carbonyl}piperazin-1-yl)-2-(trifluoromethyl)nicotinate. Yield: 23 mg (46%).

¹H NMR (400 MHz, d₆-DMSO): δ 1.27 (3H, t, J=7.1 Hz), 3.48-3.53 (4H, m), 3.70 (3H, s), 3.84-3.89 (4H, m), 4.17 (2H, d, J=5.7 Hz), 4.27 (2H, q, J=7.1 Hz), 6.82-6.86 (2H, m), 7.06 (1H, t, J=5.7 Hz), 7.15-7.20 (2H, m), 8.53 (1H, s). MS m/z: 492 (M+1).

### Example 71

### 3-{1-(anilinocarbonyl)-4-[3-cyano-5-(ethoxycarbonyl)-6-(trifluoromethyl)pyridin-2-yl]piperazin-2-yl}propanoic acid

### (a) Ethyl 6-[3-(3-tert-butoxy-3-oxopropyl)piperazin-1-yl]-5-cyano-2-(trifluoromethyl)nicotinate

Ethyl 6-chloro-5-cyano-2-(trifluoromethyl)nicotinate (250 mg, 0.90 mmol) and tert-butyl 3-piperazin-2-ylpropanoate (192 mg, 0.90 mmol) was dissolved in ethanol (2 ml). Triethylamine (0.15 ml, 1.08 mmol) was added. The solution was heated in a microwave reactor at 150° C. for 20 min. The solvent was evaporated in vacuo and the residue was dissolved in CH₂Cl₂ (50 ml). This solution washed with water (50 ml), dried (MgSO₄) and evaporated in vacuo. The residue was submitted to flash chromatography (SiO₂, CH₂Cl₂/methanol 50:1). Yield: 162 mg (40%).

¹H NMR (400 MHz, CDCl₃): δ 1.36 (3H, t, J=7.2 Hz), 1.44 (9H, s), 1.58-1.84 (3H, m), 2.35 (2H, t, J=7.7 Hz), 2.75-2.83 (1H, m), 2.85-2.93 (2H, m), 3.13 (1H, dt, J=2.7 and 12.5 Hz), 3.18-3.28 (1H, m), 4.35 (2H, q, J=7.2 Hz), 4.59-4.67 (2H, m), 8.34 (1H, s).

MS m/z: 457 (M+1).

### (b) Ethyl 6-[4-(anilinocarbonyl)-3-(3-tert-butoxy-3-oxopropyl)piperazin-1-yl]-5-cyano-2-(trifluoromethyl)nicotinate

Ethyl 6-[3-(3-tert-butoxy-3-oxopropyl)piperazin-1-yl]-5-cyano-2-(trifluoromethyl)nicotinate (21 mg, 0.046 mmol) was dissolved in CH₂Cl₂ (2 ml) under nitrogen. Phenyl isocyanate (10 μl, 0.055 mmol) was added. The solution was stirred at room temperature under nitrogen over night. PS-TRIS (50 mg, 4.1 mmol/g) was added and the stirring was continued for 5 h. The solid material was removed by filtration and the filtrate was evaporated in vacuo. Yield: 21 mg (79%).

¹H NMR (400 MHz, CDCl₃): δ 1.38 (3H, t, J=7.2 Hz), 1.51 (9H, s), 1.80-1.90 (1H, m), 1.90-2.00 (1H, m), 2.41-2.48 (2H, m), 3.24 (1H, dt, J=3.5 and 12.4 Hz), 3.53-3.64 (2H, m), 4.16-4.25 (1H, m), 4.38 (2H, q, J=7.2 Hz), 4.39-4.44 (1H, m), 4.50-4.65 (2H, m), 7.01 (1H, t, J=7.5 Hz), 7.29 (2H, t, J=8.0 Hz), 7.59 (2H, d, J=7.7 Hz), 8.38 (1H, s), 8.42 (1H, s br). MS m/z: 576 (M+1).

### (c) 3-{1-(Anilinocarbonyl)-4-[3-cyano-5-(ethoxycarbonyl)-6-(trifluoromethyl)pyridin-2-yl]piperazin-2-yl}propanoic acid

Ethyl 6-[4-(anilinocarbonyl)-3-(3-tert-butoxy-3-oxopropyl)piperazin-1-yl]-5-cyano-2-(trifluoromethyl)nicotinate (21 mg, 0.037 mmol) was dissolved in CH₂Cl₂ (4 ml). Trifluoroacetic acid (2 ml) was added. The solution was stirred at room temperature for 3.5 h. The solvents were evaporated in vacuo and the residue was coevaporated with toluene (2×3 ml). The residue was submitted to flash chromatography (SiO₂, CH₂Cl₂/methanol 12:1) to give 3-{1-(Anilinocarbonyl)-4-[3-cyano-5-(ethoxycarbonyl)-6-(trifluoromethyl)pyridin-2-yl]piperazin-2-yl}propanoic acid. Yield: 15 mg (79%).

¹H NMR (400 MHz, CDCl₃): δ 1.38 (3H, t, J=7.2 Hz), 1.81-1.92 (1H, m), 1.93-2.03 (1H, m), 2.54 (2H, t, J=5.9 Hz), 3.21-3.31 (1H, m), 3.47-3.58 (2H, m), 4.16-4.23 (1H, m), 4.23-4.30 (1H, m), 4.38 (2H, q, J=7.2 Hz), 4.48-4.55 (1H, m), 4.58-4.65 (1H, m), 7.00 (1H, t, J=7.4 Hz), 7.24 (2H, t, J=7.5 Hz), 7.44 (2H, d, J=7.7 Hz), 7.95 (1H, s), 8.37 (1H, s). MS m/z: 520 (M+1).

### Example 72

### ethyl 6-{4-[(anilinocarbonyl)amino]piperidin-1-yl}-5-chloronicotinate

### (a) Ethyl 6-{4-[(tert-butoxycarbonyl)amino]piperidin-1-yl}-5-chloronicotinate

Ethyl 5,6-dichloronicotinate (1.00 g, 4.5 mmol) and 4-(N-Boc amino)-piperidine (0.765 g, 3.8 mmol) were dissolved in CH₃CN (8 mL) at room temperature. DIPEA (1.66 g, 9.5 mmol) was added and the system heated at reflux for 16 h. The reaction mixture was cooled to room temperature and the solvent concentrated under reduced pressure. The material was partitioned between EtOAc (50 mL) and saturated aqueous NH₄Cl (2×30 mL). The organics were washed with brine (30 mL), dried (MgSO₄) and concentrated under reduced pressure to afford the crude product. Flash chromatography (6:1 hexanes/EtOAc) gave ethyl 6-{4-[(tert-butoxycarbonyl)amino]piperidin-1-yl}-5-chloronicotinate. Yield: 1.04 g (84%).

¹H NMR (400 MHz, CDCl₃): δ 1.38 (3H, t, J=7.0 Hz), 1.46 (9H, s), 2.01-2.12 (2H, m), 3.04 (2H, m), 3.64-3.78 (1H, s), 4.02-4.06 (2H, m), 4.36 (2H, q, J=7.0 Hz), 4.50-4.52 (1H, m), 8.11 (1H, s), 8.73 (1H, s). MS m/z: 384 (M+1).

### (b) Ethyl 6-(4-aminopiperidin-1-yl)-5-chloronicotinate dihydrochloride

Ethyl 6-{4-[(tert-butoxycarbonyl)amino]piperidin-1-yl}-5-chloronicotinate (1.00 g, 2.8 mmol) was dissolved in DCM (2 mL) at room temperature. HCl (3.50 mL, 14 mmol) was added and the system stirred for 16 h. The solvent was concentrated under reduced pressure. The material was azeotroped using hexanes and toluene, and concentrated under reduced pressure to afford ethyl 6-(4-aminopiperidin-1-yl)-5-chloronicotinate dihydrochloride product as a solid. Yield: 1.00 g (91%).

¹H NMR (400 MHz, CD₃OD): δ 1.38 (3H, t, J=7.1 Hz), 1.76-1.86 (2H, m), 2.13-2.16 (2H, m), 3.11-3.18 (2H, m), 3.40-3.46 (1H, m), 4.21-4.25 (2H, m), 4.37 (2H, q, J=7.1 Hz), 8.28 (1H, s), 8.68 (1H, s). MS m/z: 284 (M+1).

### (c) ethyl 6-{4-[(anilinocarbonyl)amino]piperidin-1-yl}-5-chloronicotinate

Ethyl 6-(4-aminopiperidin-1-yl)-5-chloronicotinate dihydrochloride (0.100 g, 0.254 mmol) and TEA (0.177 mL, 1.27 mmol) were dissolved in CH₂Cl₂ (1 mL), at room temperature. Phenyl isocyanate (0.031 mL, 0.280 mmol), was added and the system stirred for 1 h. DCM (30 mL) was added and the combined organics were washed with saturated NH₄Cl (2×20 mL) and brine (1×20 mL). The organics were then dried (MgSO₄) and concentrated under reduced pressure. Trituration (50% Et₂O in Hexanes) afforded ethyl 6-{4-[(anilinocarbonyl)amino]piperidin-1-yl}-5-chloronicotinate product as a solid. Yield: 0.078 g (76%).

¹H NMR (400 MHz, CDCl₃): δ 1.38 (3H, t, J=7.0 Hz), 1.47-1.66 (2H, m), 2.04-2.15 (2H, m), 3.00-3.13 (2H, m), 3.92-4.09 (3H, m), 4.36 (2H, q, J=7.0 Hz), 4.76-4.86 (1H, m), 7.07-7.15 (1H, m), 7.24-7.37 (5H, m), 8.10 (1H, s), 8.72 (1H, s). MS m/z: 403 (M+1).

### Example 73

### ethyl 6-{3-[(anilinocarbonyl)amino]azetidin-1-yl}-5-chloronicotinate

### (a) Ethyl 6-(3-(tert-butoxycarbonylamino)azetidin-1-yl)-5-chloronicotinate

Ethyl 5,6-dichloronicotinate (0.630 g, 2.86 mmol), tert-butyl azetidin-3-ylcarbamate (0.591 g, 3.43 mmol), and DIEA (1.66 g, 9.5 mmol), were dissolved in DMA (10 mL), and the system heated at 120° C. for 16 h. The reaction mixture was cooled to room temperature and the solvent concentrated under reduced pressure. EtOAc (100 mL) was added and the combined organics were washed with a 50% mixture of saturated aqueous NH₄Cl in brine (80 mL), dried (MgSO₄) and concentrated under reduced pressure. Flash chromatography (20% EtOAc in Hexanes), gave ethyl 6-(3-(tert-butoxycarbonylamino)azetidin-1-yl)-5-chloronicotinate. Yield: 0.510 g (50%).

¹H NMR (400 MHz, CDCl₃): δ 1.37 (3H, t, J=7.1 Hz), 1.46 (9H, s), 4.10-4.17 (2H, m), 4.34 (2H, q, J=7.1 Hz), 4.51-4.73 (3H, m), 4.96-5.05 (1H, m), 7.98 (1H, s), 8.65 (1H, s). MS m/z: 384 (M+1).

### (b) Ethyl 6-(3-aminoazetidin-1-yl)-5-chloronicotinate dihydrochloride

Ethyl 6-(3-(tert-butoxycarbonylamino)azetidin-1-yl)-5-chloronicotinate (0.510 g, 1.43 mmol) was dissolved in DCM (4 mL). HCl (4 M in dioxane, 1.80 mL, 7.17 mmol) was added slowly. The reaction mixture was stirred at room temperature for 16 h. The reaction mixture was concentrated under reduced pressure and azeotroped (Hexane, Toluene), to afford ethyl 6-(3-aminoazetidin-1-yl)-5-cyano-2-methylnicotinate dihydrochloride as a solid, which was used crude assuming a 100% conversion.

¹H NMR (400 MHz, CD₃OD): δ 1.33-1.42 (3H, m), 4.22-4.42 (3H, m), 4.55-4.66 (2H, m), 8.25 (1H, s), 8.54 (1H, s).

### (c) ethyl 6-{3-[(anilinocarbonyl)amino]azetidin-1-yl}-5-chloronicotinate

Ethyl 6-(4-aminopiperidin-1-yl)-5-chloronicotinate dihydrochloride (0.100 g, 0.254 mmol) and TEA (0.177 mL, 1.27 mmol) were dissolved in CH₂Cl₂ (1 mL), at room temperature. Phenyl isocyanate (0.031 mL, 0.280 mmol), was slowly added and the system stirred for 1 h at room temperature. DCM (30 mL) was added and the combined organics were washed with saturated NH₄Cl (2×20 mL) and brine (1×20 mL). The organics were then dried (MgSO₄) and concentrated under reduced pressure. Trituration (50% Et₂O in Hexanes) ethyl 6-{3-[(anilinocarbonyl)amino]azetidin-1-yl}-5-chloronicotinate product as a solid. Yield: 0.078 g (76.0%).

¹H NMR (400 MHz, CDCl₃): δ 1.36 (3H, t, J=6.7 Hz), 4.08-4.16 (2H, m), 4.33 (2H, q, J=6.7 Hz), 4.66-4.79 (3H, m), 5.09-5.18 (1H, m), 6.32 (1H, s), 7.10-7.19 (1H, m), 7.23-7.40 (4H, m), 7.98 (1H, s), 8.64 (1H, s). MS m/z: 375 (M+1).

### Example 74

### ethyl 6-(3-{[(anilinocarbonyl)amino]methyl}azetidin-1-yl)-5-cyano-2-methylnicotinate

### (a) Ethyl 2-((dimethylamino)methylene)-3-oxobutanoate

Ethyl 3-oxobutanoate (250 mL, 1961 mmol) was stirred at r.t and 1,1-dimethoxy-N,N-dimethylmethanamine (327 mL, 2452 mmol) was added drop-wise. The reaction mixture was allowed to stir at r.t overnight. The reaction mixture was concentrated under vacuum and then azeotroped with toluene (3×300 mL) and placed under high vacuum to afford ethyl 2-((dimethylamino)methylene)-3-oxobutanoate as an oil, which was used without further purification. Yield: 363 g (100%). MS m/z: 186 (M+1).

### (b) Ethyl 5-cyano-2-methyl-6-oxo-1,6-dihydropyridine-3-carboxylate

2-Cyanoacetamide (33.0 g, 392 mmol) was suspended in THF (250 mL) and slowly added to a suspension of NaH (60% dispersion in mineral oil, 16.5 g, 412 mmol) in THF (500 mL). The mixture was stirred for 2 h at r.t followed by the drop-wise addition of ethyl 2-((dimethylamino)methylene)-3-oxobutanoate (72.6 g, 392 mmol) suspended in THF (250 mL). The reaction mixture was stirred at r.t for 16 h and then acidified to pH 6 with acetic acid. Concentration under reduced pressure afforded crude material, which was suspended in 1 N HCl (1 L) and stirred for 30 minutes. The suspension was filtered and the product collected as a solid, which was azeotroped with Toluene (3×1 L) to afford ethyl 5-cyano-2-methyl-6-oxo-1,6-dihydropyridine-3-carboxylate as a solid. Yield: 75.3 g (93%).

¹H NMR (400 MHz, DMSO-d₆): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylnicotinate

Ethyl 5-cyano-2-methyl-6-oxo-1,6-dihydropyridine-3-carboxylate (70.33 g, 341 mmol) was suspended in phosphoryl trichloride (124.5 mL, 1364 mmol) and the system heated at 100° C. overnight. The reaction mixture was cooled to r.t and concentrated under reduced pressure. The residue was diluted with DCM and poured onto ice. The bi-phasic mixture was stirred at r.t and slowly quenched with solid K₂CO₃ until all the POCl₃ had hydrolysed. The aqueous phase was extracted into DCM and the organics, dried (MgSO₄) and passed through a silica plug. The organics were concentrated under reduced pressure to afford ethyl 6-chloro-5-cyano-2-methylnicotinate as a solid, which was used without further purification. Yield: 61 g (80%).

¹H NMR (400 MHz, CDCl₃): δ 1.42 (3H, t, J=7.1 Hz), 2.91 (3H, s), 4.40 (2H, q, J=7.1 Hz), 8.49 (1H, s).

### (d) Ethyl 6-(3-((tert-butoxycarbonylamino)methyl)azetidin-1-yl)-5-cyano-2-methylnicotinate

Ethyl 6-chloro-5-cyano-2-methylnicotinate (1.00 g, 4.5 mmol), tert-butyl azetidin-3-ylmethylcarbamate (0.99 g, 5.30 mmol), and DIPEA (3.90 mL, 22.0 mmol) were dissolved in DCM (20 mL) and stirred at room temperature for 2 h. The reaction mixture was concentrated under reduced pressure and diluted with EtOAc (40 mL). The combined organics were washed with saturated NH₄Cl (2×30 mL), H₂O (1×20 mL), brine (1×30 mL), dried (MgSO₄) and concentrated under reduced pressure to afford the crude product. Flash chromatography (25 to 35% EtOAc in hexanes) gave ethyl 6-(3-((tert-butoxycarbonylamino)methyl)azetidin-1-yl)-5-cyano-2-methylnicotinate as a solid. Yield: 1.49 g (90%).

¹H NMR (400 MHz, CDCl₃): δ 1.37 (3H, t, J=7.2 Hz), 1.45 (9H, s), 2.70 (3H, s), 2.88-2.99 (1H, m), 3.35-3.46 (2H, m), 4.02-4.14 (2H, m), 4.30 (2H, q, J=7.2 Hz), 4.39-4.50 (2H, m), 4.64-4.76 (1H, m), 8.26 (1H, s). MS m/z: 375 (M+1).

### (e) Ethyl 6-(3-(aminomethyl)azetidin-1-yl)-5-cyano-2-methylnicotinate dihydrochloride

Ethyl 6-(3-((tert-butoxycarbonylamino)methyl)azetidin-1-yl)-5-cyano-2-methylnicotinate (1.50 g. 4.00 mmol) was dissolved HCl (4 M, 20.0 mL, 80.0 mmol). The reaction mixture was stirred at room temperature for 16 h and concentrated under reduced pressure to yield ethyl 6-(3-(aminomethyl)azetidin-1-yl)-5-cyano-2-methylnicotinate dihydrochloride as a solid, which was used crude assuming 100% conversion.

¹H NMR (400 MHz, CDCl₃): δ 1.30 (3H, t, J=7.1 Hz), 2.60 (3H, s), 2.94-3.05 (1H, m), 3.10-3.20 (2H, m), 4.11-4.19 (2H, m), 4.23 (2H, q, J=7.1 Hz), 4.34-4.57 (2H, m), 7.93-8.04 (2H, m), 8.29 (1H, s). MS m/z: 275 (M+1).

### (f) ethyl 6-(3-{[(anilinocarbonyl)amino]methyl}azetidin-1-yl)-5-cyano-2-methylnicotinate

Ethyl 6-(3-(aminomethyl)azetidin-1-yl)-5-cyano-2-methylnicotinate dihydrochloride (0.200 g, 0.580 mmol), phenyl isocyanate (0.076 mL, 0.690 mmol) and DIEA (0.500 mL, 2.90 mmol) were dissolved in DCM (10 mL) and stirred at room temperature for 3 h. DCM (50 mL) added and the combined organics were washed with saturated NaHCO₃ (2×40 mL), dried (MgSO4) and concentrated under reduced pressure to afford the crude product. Trituration (DCM) gave ethyl 6-(3-{[(anilinocarbonyl)amino]methyl}azetidin-1-yl)-5-cyano-2-methylnicotinate as a solid. Yield: 0.145 g (64%)

¹H NMR (400 MHz, d6-DMSO): δ 1.29 (3H, t, J=7.1 Hz), 2.60 (3H, s), 2.89-2.93 (1H, m), 3.28-3.42 (2H, m), 4.01-4.12 (2H, m), 4.22 (2H, q, J=7.1 Hz), 4.31-4.44 (2H, m), 6.38-6.46 (1H, m), 6.84-6.94 (1H, m), 7.17-7.26 (2H, m), 7.34-7.43 (2H, m), 8.26 (1H, s), 8.46 (1H, m). MS m/z: 394 (M+1).

### Example 75

### ethyl 6-[3-({[(benzylamino)carbonyl]amino}methyl)azetidin-1-yl]-5-cyano-2-methylnicotinate

Ethyl 6-(3-(aminomethyl)azetidin-1-yl)-5-cyano-2-methylnicotinate dihydrochloride (0.200 g, 0.580 mmol), benzyl isocyanate (0.085 mL, 0.690 mmol) and DIEA (0.500 mL, 2.90 mmol) were dissolved in DCM (10 mL) and stirred at room temperature for 3 h. DCM (50 mL) added and the combined organics were washed with saturated NaHCO₃ (2×40 mL), dried (MgSO4) and concentrated under reduced pressure to afford the crude product. Trituration (DCM) gave ethyl 6-[3-({[(benzylamino)carbonyl]amino}methyl)azetidin-1-yl]-5-cyano-2-methylnicotinate as a solid. Yield: 0.213 g (91%)

¹H NMR (400 MHz, d6-DMSO): δ 1.30 (3H, t, J=7.1 Hz), 2.60 (3H, s), 2.77-2.90 (1H, m), 3.97-4.11 (2H, m), 4.16-4.27 (4H, m), 4.28-4.43 (2H, m), 6.33-6.42 (1H, m), 7.17-7.32 (5H, m), 8.26 (1H, s). MS m/z: 408 (M+1).

### Example 76

### ethyl 6-{3-[(anilinocarbonyl)amino]azetidin-1-yl}-5-cyano-2-methylnicotinate

### (a) Ethyl 6-{3-[(tert-butoxycarbonyl)amino]azetidin-1-yl}-5-cyano-2-methylnicotinate

Ethyl 6-chloro-5-cyano-2-methylnicotinate (6.20 g, 29.4 mmol), tert-butyl azetidin-3-ylcarbamate (5.07 g, 29.4 mmol), and DIPEA (5.13 mL, 29.4 mmol) were dissolved in DCE (40 mL) and stirred at r.t for 1 h. The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Flash chromatography (1:6 EtOAc/hexanes) gave ethyl 6-{3-[(tert-butoxycarbonyl)amino]azetidin-1-yl}-5-cyano-2-methylnicotinate as a solid. Yield: 7.00 g (66%)

¹H NMR (400 MHz, CDCl₃): δ 1.37 (3H, t, J=7.2 Hz), 1.46 (9H, s), 2.70 (1H, s), 4.18-4.22 (2H, m), 4.30 (2H, q, J=7.2 Hz), 4.59 (1H, s), 4.67-4.72 (2H, m), 5.00 (1H, s), 8.26 (1H, s). MS m/z: 361 (M+1).

### (b) Ethyl 6-(3-aminoazetidin-1-yl)-5-cyano-2-methylnicotinate bis(trifluoroacetate)

Ethyl 6-{3-[(tert-butoxycarbonyl)amino]azetidin-1-yl}-5-cyano-2-methylnicotinate (1.00 g, 2.77 mmol) was dissolved in DCM (10 mL). TFA (6.40 mL, 83.2 mmol) was added slowly. The reaction mixture was stirred at r.t for 30 minutes. The mixture was concentrated under reduced pressure to afford ethyl 6-(3-aminoazetidin-1-yl)-5-cyano-2-methylnicotinate bis(trifluoroacetate) as a solid, which was used crude assuming a 100% conversion.

### (c) ethyl 6-{3-[(anilinocarbonyl)amino]azetidin-1-yl}-5-cyano-2-methylnicotinate

Ethyl 5-cyano-6-(1,4-diazepan-1-yl)-2-methylnicotinate (0.100 g, 0.35 mmol) was dissolved in DCM (2 mL) and DIEA (0.30 mL, 1.7 mmol) was added. Benzenesulfonyl isocyanate (0.046 mL, 0.35 mmol) was added and the reaction mixture was stirred at room temperature for 16 h. The reaction mixture was concentrated under reduced pressure, diluted with EtOAc (40 mL) and washed with saturated aqueous NH₄Cl (2×25 mL) and brine (25 mL). The organics were dried (MgSO₄) and concentrated under reduced pressure to afford the crude product. Trituration (50% EtOAc in hexanes) gave ethyl 5 ethyl 6-{3-[(anilinocarbonyl)amino]azetidin-1-yl}-5-cyano-2-methylnicotinate as a solid. Yield: 0.077 g (61%).

¹H NMR (400 MHz, d₆-DMSO): δ 1.30 (3H, t, J=7.1 Hz), 2.62 (3H, s), 4.11-4.29 (4H, m), 4.52-4.64 (3H, m), 6.82-6.95 (2H, m), 7.18-7.27 (2H, m), 7.37-7.43 (2H, m), 8.30 (1H, m), 8.62 (1H, s). MS m/z: 380 (M+1).

### Example 77

### ethyl 6-(3-{[(benzylamino)carbonyl]amino}azetidin-1-yl)-5-cyano-2-methylnicotinate

Ethyl 6-(3-aminoazetidin-1-yl)-5-cyano-2-methylnicotinate bistrifluoroacetate (0.151 g, 0.333 mmol) and DIEA (0.290 mL, 1.66 mmol) were dissolved in CH₂Cl₂ (2 mL), at room temperature. Phenyl isocyanate (0.041 mL, 0.333 mmol), was slowly added and the system stirred for 16 h at room temperature. DCM (30 mL) was added and the combined organics were washed with saturated NaHCO₃ (2×30 mL). The organics were then dried (MgSO₄) and concentrated under reduced pressure. Trituration (50% EtOAc in Hexanes) afforded ethyl 6-(3-{[(benzylamino)carbonyl]amino}azetidin-1-yl)-5-cyano-2-methylnicotinate product as a solid. Yield: 0.076 g (58%).

¹H NMR (400 MHz, d₆-DMSO): δ 1.29 (3H, t, J=6.7 Hz), 2.61 (3H, s), 4.07-4.16 (2H, m), 4.18-4.27 (4H, m), 4.46-4.60 (3H, m), 6.55-6.62 (1H, m), 6.70-6.76 (1H, m), 7.18-7.35 (5H, m), 8.28 (1H, s). MS m/z: 394 (M+1).

### Example 78

### ethyl 6-{4-[(benzoylamino)carbonothioyl]piperazin-1-yl}-5-chloronicotinate

Ethyl 5-chloro-6-piperazin-1-ylnicotinate (50 mg, 0.19 mmol) was dissolved in dry THF (1 mL) under inert atmosphere and was cooled to 0° C. Benzoyl isothiocyanate (30 mg, 0.19 mmol) was added and the temperature was allowed to take r.t. followed by stirring for 50 h. at that temperature. The reaction mixture was added PS-trisamin, stirred for 1 h and filtered. The reaction mixture was purified by preparative HPLC (C8 25×300, 0.1 M NH4Ac/MeCN, gradient) to give ethyl 6-{4-[(benzoylamino)carbonothioyl]piperazin-1-yl}-5-chloronicotinate. Yield=35 mg (44%).

¹H NMR (400 MHz, d₆-DMSO): δ 1.38 (3H, t, J=7.1), 3.70-3.90 (8H, m), 4.37 (2H, q, J=7.1), 7.46-7.53 (2H, m), 7.57-7.63 (1H, m), 7.83-7.89 (2H, m), 8.16 (1H, d, J=2.0), 8.54 (1H, br s), 8.75 (1H, d, J=2.0) MS m/z: 433 (M+1).

### Example 79

### ethyl 5-cyano-2-methyl-6-(3-{[(phenylacetyl)amino]methyl}azetidin-1-yl)nicotinate

Ethyl 6-(3-(aminomethyl)azetidin-1-yl)-5-cyano-2-methylnicotinate dihydrochloride (0.200 g, 0.580 mmol), phenyl acetyl chloride (0.092 mL, 0.690 mmol) and DIEA (0.500 mL, 2.90 mmol) were dissolved in DCM (10 mL) and stirred at room temperature for 3 h. DCM (50 mL) added and the combined organics were washed with saturated NH₄Cl (2×40 mL), dried (MgSO4) and concentrated under reduced pressure to afford the crude product. Flash chromatography (0 to 2.5% MeOH in DCM) gave ethyl 5-cyano-2-methyl-6-(3-{[(phenylacetyl)amino]methyl}azetidin-1-yl)nicotinate as a solid. Yield: 0.217 g (96%)

¹H NMR (400 MHz, d6-DMSO): δ 1.30 (3H, t, J=7.1 Hz), 2.60 (3H, s), 2.81-2.89 (1H, m), 3.41 (2H, m), 3.97-4.06 (2H, m), 4.23 (2H, q, J=7.1 Hz), 4.27-4.36 (2H, m), 7.14-7.25 (5H, m), 8.25-8.33 (2H, m). MS m/z: 393 (M+1).

### Example 80

### ethyl 6-{3-[(benzoylamino)methyl]azetidin-1-yl}-5-cyano-2-methylnicotinate

Ethyl 6-(3-(aminomethyl)azetidin-1-yl)-5-cyano-2-methylnicotinate dihydrochloride (0.200 g, 0.580 mmol), benzoyl chloride (0.080 mL, 0.690 mmol) and DIEA (0.500 mL, 2.90 mmol) were dissolved in DCM (10 mL) and stirred at room temperature for 3 h. DCM (50 mL) added and the combined organics were washed with saturated NaHCO₃ (2×40 mL), dried (MgSO4) and concentrated under reduced pressure to afford the crude product. Flash chromatography (0 to 2.5% MeOH in DCM) gave ethyl 6-{3-[(benzoylamino)methyl]azetidin-1-yl}-5-cyano-2-methylnicotinate as a solid. Yield: 0.202 g (93%).

¹H NMR (400 MHz, d6-DMSO): δ 1.29 (3H, t, J=7.1 Hz), 2.59 (3H, s), 2.89-3.03 (1H, m), 3.52-3.59 (2H, m), 4.07-4.19 (2H, m), 4.23 (2H, q, J=7.1 Hz), 4.33-4.44 (2H, m), 7.42-7.55 (3H, m), 7.79-7.85 (2H, m), 8.26 (1H, s), 8.65-8.72 (1H, m). MS m/z: 379 (M+1).

### Example 81

### ethyl 6-[4-(2-anilino-2-oxoethyl)piperidin-1-yl]-5-cyano-2-methylnicotinate

### (a) 2-(1-(3-cyano-5-(ethoxycarbonyl)-6-methylpyridin-2-yl)piperidin-4-yl)acetic acid

Ethyl 6-chloro-5-cyanonicotinate (0.500 g, 2.4 mmol), 2-(piperidin-4-yl)acetic acid (0.410 g, 2.80 mmol), and DIPEA (2.10 mL, 12.0 mmol) were dissolved in DCM (4 mL) and stirred at room temperature for 8 h. The reaction mixture was cooled to room temperature and the solvent concentrated under reduced pressure. The material was partitioned between EtOAc (50 mL) and saturated aqueous NaHCO₃ (2×30 mL). The organics were washed with brine (30 mL), dried (MgSO₄) and concentrated under reduced pressure to afford the crude product. No purification was done.

¹H NMR (400 MHz, CDCl₃): δ 1.34-1.42 (5H, m), 1.87-1.98 (2H, m), 2.08-2.22 (1H, m), 2.31-2.38 (2H, m), 2.71 (3H, s), 3.03-3.15 (2H, m), 4.31 (2H, q, J=7.1 Hz), 4.71-4.81 (2H, m), 8.34 (1H, s). MS m/z: 332 (M+1).

### (b) ethyl 6-[4-(2-anilino-2-oxoethyl)piperidin-1-yl]-5-cyano-2-methylnicotinate

2-(1-(3-cyano-5-(ethoxycarbonyl)-6-methylpyridin-2-yl)piperidin-4-yl)acetic acid (0.100 g, 0.302 mmol), EDCI (0.069 g, 0.360 mmol) and HOBT (0.049 g, 0.360 mmol) were dissolved in DCM (2 mL) at room temperature. The reaction mixture was stirred at room temperature for 10 minutes and then aniline (0.033 mL, 0.360 mmol) and DIEA (0.160 mL, 0.91 mmol) were added. The reaction mixture was stirred at room temperature for 18 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH), gave ethyl 6-[4-(2-anilino-2-oxoethyl)piperidin-1-yl]-5-cyano-2-methylnicotinate as a solid. Yield: 0.096 g (78.0%).

¹H NMR (400 MHz, CDCl₃): δ 1.32-1.46 (5H, m), 1.89-2.01 (2H, m), 2.23-2.37 (3H, m), 2.71 (3H, s), 3.02-3.15 (2H, m), 4.26-4.37 (2H, q, J=7.1 Hz), 4.71-4.81 (2H, m), 7.08-7.17 (2H, m), 7.28-7.38 (2H, m), 7.47-7.55 (2H, m). MS m/z: 407 (M+1).

### Example 82

### ethyl 6-{4-[2-(benzylamino)-2-oxoethyl]piperidin-1-yl}-5-cyano-2-methylnicotinate

2-(1-(3-cyano-5-(ethoxycarbonyl)-6-methylpyridin-2-yl)piperidin-4-yl)acetic acid (0.100 g, 0.302 mmol), EDCI (0.069 g, 0.360 mmol) and HOBT (0.049 g, 0.360 mmol) were dissolved in DCM (2 mL) at room temperature. The reaction mixture was stirred at room temperature for 10 minutes and then benzylamine (0.040 mL, 0.360 mmol) and DIEA (0.160 mL, 0.91 mmol) were added. The reaction mixture was stirred at room temperature for 18 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH), gave ethyl 6-(4-(2-(benzylamino)-2-oxoethyl)piperidin-1-yl)-5-cyano-2-methylnicotinate as a solid. Yield: 0.079 g (62.0%).

¹H NMR (400 MHz, CDCl₃): δ 1.23-1.42 (5H, m), 1.85-1.95 (2H, m), 2.14-2.20 (2H, m), 2.21-2.30 (1H, m), 2.71 (3H, s), 3.01-3.13 (2H, m), 4.32 (2H, q, J=7.1 Hz), 4.44-4.50 (2H, m), 4.71-4.80 (2H, m), 5.66-5.73 (1H, m), 7.24-7.40 (5H, m), 8.34 (1H, m).

MS m/z: 421 (M+1).

### Example 83

### N-({1-[3-cyano-5-(ethoxycarbonyl)-6-methylpyridin-2-yl]azetidin-3-yl}carbonyl)phenylalanine

### (a) 1-[3-Cyano-5-(ethoxycarbonyl)-6-methylpyridine-2-yl]azetidine-3-carboxylic acid

Ethyl 6-chloro-5-cyano-2-methylnicotinate (50.98 g, 227 mmol), azetidine-3-carboxylic acid (24.09 g, 238 mmol) and DIPEA (118.9 mL, 681 mmol) were suspended in EtOH (250 mL) and heated at reflux for 1 h. The reaction mixture was cooled to r.t and added drop-wise to KHSO₄ (154.5 g, 1135 mmol) in water (3000 mL). The solids were collected by filtration and dried under vacuum to afford 1-[3-Cyano-5-(ethoxycarbonyl)-6-methylpyridine-2-yl]azetidine-3-carboxylic acid as a solid, which was used without further purification. Yield: 65.33 g (100%).

¹H NMR (400 MHz, CDCl₃): δ 1.37 (3H, t, J=7.1 Hz), 2.72 (3H, s), 3.59-3.68 (1H, m), 4.31 (2H, q, J=7.1 Hz), 4.55-4.68 (4H, m), 8.28 (1H, s). MS m/z: 290 (M+1).

### (b) N-({1-[3-cyano-5-(ethoxycarbonyl)-6-methylpyridin-2-yl]azetidin-3-yl}carbonyl)phenylalanine

HATU (19 mg, 0.05 mmol) and DIPEA (32 mg, 0.250 mmol) were added to a stirred solution of 1-[3-Cyano-5-(ethoxycarbonyl)-6-methylpyridine-2-yl]azetidine-3-carboxylic acid (14 mg, 0.05 mmol) in DMF (0.5 mL) and the stirring was continued for 0.5 hours at r.t. Phenylalanine (12 mg, 0.075 mmol) was added and the mixture was stirred at r.t for 16 hours. Another equivalent of HATU (19 mg, 0.05 mmol) was added and stirring at rt was continued for 16 h. LC/MS showed 40% product and 27% A. Another eq. HATU (19 mg, 0.05 mmol) and the stirring was continued for another 16 h further. Purification by preparative HPLC was performed using Waters Fraction Lynx Purification System with Kromasil C8 5 mm 20×100 mm column. The mobile phase used was varying gradients of acetonitrile and 0.1 M ammonium acetate buffer. MS triggered fraction collection was used. Yield 8 mg (36%).

¹H NMR (400 MHz, d6-DMSO): δ 1.31 (t, J=6.9 Hz, 3H), 2.63 (s, 3H), 2.83-2.91 (m, 2H), 3.09-3.17 (m, 2H), 3.42-3.55 (m, 2H), 4.04-4.11 (m, 1H), 4.25 (q, J=6.9 Hz, 2H), 4.33-4.45 (m, 2H), 7.16-7.29 (m, 5H), 8.20-8.26 (m, 1H), 8.29 (s, 1H). MS m/z: 437 (M+1).

### Example 84

### ethyl 5-chloro-6-(4-{[(2,4,5-trichlorophenyl)amino]carbonyl}piperazin-1-yl)nicotinate

Prepared in according to method A from ethyl 5-chloro-6-piperazin-1-ylnicotinate and 1,2,4-trichloro-5-isocyanatobenzene to give ethyl 5-chloro-6-(4-{[(2,4,5-trichlorophenyl)amino]carbonyl}piperazin-1-yl)nicotinate. Yield: 23.6 mg (48%).

¹H NMR (400 MHz, d₆-DMSO): δ 1.29 (3H, t, J=7.1 Hz), 3.48-3.55 (4H, m), 3.56-3.62 (4H, m), 4.29 (2H, q, J=7.1 Hz), 7.70 (2H, s), 8.11 (1H, d, J=2.0 Hz), 8.56 (1H, s), 8.68 (1H, d, J=2.0 Hz). MS m/z: 493 (M+1).

### Example 85

### ethyl 6-{4-[(1,3-benzodioxol-5-ylamino)carbonyl]piperazin-1-yl}-5-cyano-2-(trifluoromethyl)nicotinate

Can be prepared in according to method A from ethyl 5-cyano-6-piperazin-1-yl-2-(trifluoromethyl)nicotinate and 5-isocyanato-1,3-benzodioxole to give ethyl 6-{4-[(1,3-benzodioxol-5-ylamino)carbonyl]piperazin-1-yl}-5-cyano-2-(trifluoromethyl)nicotinate

### Example 86

### ethyl 5-cyano-6-(4-{[(4-isopropylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-4-isopropylbenzene to give ethyl 5-cyano-6-(4-{[(4-isopropylphenyl)amino]carbonyl}piperazin-1-yl)-2-(trifluoromethyl)nicotinate. Yield: 8.4 mg (17%).

¹H NMR (400 MHz, d₆-DMSO): δ 1.15 (6H, d, J=6.9 Hz), 1.28 (3H, t, J=7.1 Hz), 2.77-2.81 (1H, m), 3.60-3.66 (4H, m), 3.90-3.96 (4H, m), 4.27 (2H, q, J=7.1 Hz), 7.09 (2H, d, J=8.5 Hz), 7.34 (2H, d, J=8.5 Hz), 8.47 (1H, s), 8.55 (1H, s). MS m/z: 491 (M+1).

### Example 87

### ethyl 5-cyano-6-(4-{[(2-phenylethyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and (2-isocyanatoethyl)benzene to give ethyl 5-cyano-6-(4-{[(2-phenylethyl)amino]carbonyl}piperazin-1-yl)-2-(trifluoromethyl)nicotinate.

Yield: 29.6 mg (62%).

¹H NMR (400 MHz, d₆-DMSO): δ 1.27 (3H, t, J=7.1 Hz), 2.68-2.75 (2H, m), 3.20-3.28 (2H, m), 3.43-3.50 (4H, m), 3.81-3.88 (4H, m), 4.27 (2H, q, J=7.1 Hz), 6.64-6.70 (1H, m), 7.14-7.20 (3H, m), 7.23-7.29 (2H, m), 8.54 (1H, s). MS m/z: 476 (M+1).

### Example 88

### ethyl 6-{4-[(benzylamino)carbonyl]-1,4-diazepan-1-yl}-5-cyano-2-methylnicotinate

Ethyl 5-cyano-6-(1,4-diazepan-1-yl)-2-methylnicotinate (0.100 g, 0.35 mmol) was dissolved in DCM (2 mL) and DIEA (0.30 mL, 1.7 mmol) was added. (isocyanatomethyl)benzene (0.046 mL, 0.35 mmol) was added and the reaction mixture was stirred at room temperature for 16 h. The reaction mixture was concentrated under reduced pressure, diluted with EtOAc (40 mL) and washed with saturated aqueous NH₄Cl (2×25 mL) and brine (25 mL). The organics were dried (MgSO₄) and concentrated under reduced pressure to afford the crude product. Flash chromatography (40% EtOAc in hexanes with 0.5% AcOH) gave ethyl 6-{4-[(benzylamino)carbonyl]-1,4-diazepan-1-yl}-5-cyano-2-methylnicotinate as a solid. Yield: 0.116 g (79.0%).

¹H NMR (400 MHz, CDCl₃): δ 1.38 (3H, t, J=7.1 Hz), 2.00-2.12 (2H, m), 2.69 (3H, s), 3.36 (2H, m), 3.64-3.74 (2H, m), 3.98-4.14 (4H, m), 4.32 (2H, q, J=7.1 Hz), 4.37-4.46 (2H, m), 4.65-4.74 (1H, m), 7.18-7.37 (5H, m), 8.33 (1H, s). MS m/z: 422 (M+1).

### Example 89

### ethyl 5-chloro-6-[4-({[(1R,2R)-2-phenylcyclopropyl]amino}carbonyl)piperazin-1-yl]nicotinate

Prepared in according to method A from ethyl 5-chloro-6-piperazin-1-ylnicotinate and [(1R,2R)-2-isocyanatocyclopropyl]benzene give ethyl 5-chloro-6-[4-({[(1R,2R)-2-phenylcyclopropyl]amino}carbonyl)piperazin-1-yl]nicotinate. Yield: 29.9 mg (69%).

¹H NMR (400 MHz, d₆-DMSO): δ 1.29 (3H, t, J=7.1 Hz), 1.03-1.10 (1H, m), 1.12-1.18 (1H, m), 1.28 (3H, t, J=7.1 Hz), 1.83-1.91 (1H, m), 2.66-2.72 (1H, m), 3.39-3.47 (8H, m), 4.28 (2H, q, J=7.1 Hz), 6.86-6.89 (1H, m), 7.06-7.14 (3H, m), 7.19-7.25 (2H, m), 8.07-8.09 (1H, m), 8.65-8.67 (1H, m). MS m/z: 430 (M+1).

### Example 90

### ethyl 5-cyano-6-(4-{[(3,4-difluorophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1,2-difluoro-4-isocyanatobenzene to give Ethyl 5-cyano-6-(4-{[(3,4-difluorophenyl)amino]carbonyl}piperazin-1-yl)-2-(trifluoromethyl)nicotinate. Yield: 32.5 mg (67%).

¹H NMR (400 MHz, d₆-DMSO): δ 1.28 (3H, t, J=7.1 Hz), 3.61-3.67 (4H, m), 3.91-3.97 (4H, m), 4.28 (2H, q, J=7.1 Hz), 7.19-7.33 (2H, m), 7.57-7.66 (1H, m), 8.56 (1H, s), 8.77 (1H, s). MS m/z: 484 (M+1).

### Example 91

### ethyl 5-cyano-6-(4-{[(2-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-2-methylbenzene to give ethyl 5-cyano-6-(4-{[(2-methylphenyl)amino]carbonyl}piperazin-1-yl)-2-(trifluoromethyl)nicotinate. Yield: 24.9 mg (54%).

¹H NMR (400 MHz, d₆-DMSO): δ 1.28 (3H, t, J=7.1 Hz), 2.16 (3H, s), 3.60-3.67 (4H, m), 3.91-3.97 (4H, m), 4.28 (2H, q, J=7.1 Hz), 7.00-7.22 (4H, m), 8.08 (1H, s), 8.56 (1H, s). MS m/z: 462 (M+1).

### Example 92

### ethyl 5-cyano-6-(4-{[(4-ethoxyphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-ethoxy-4-isocyanatobenzene to give ethyl 5-cyano-6-(4-{[(4-ethoxyphenyl)amino]carbonyl}piperazin-1-yl)-2-(trifluoromethyl)nicotinate. Yield: 48 mg (97%).

¹H NMR (400 MHz, d₆-DMSO): δ 1.28 (3H, t, J=7.1 Hz), 3.59-3.65 (4H, m), 3.90-3.98 (6H, m), 4.28 (2H, q, J=7.1 Hz), 6.77-6.82 (2H, m), 7.29-7.34 (2H, m), 8.39 (1H, s), 8.56 (1H, s). MS m/z: 492 (M+1).

### Example 93

### ethyl 5-cyano-6-[4-({[4-(methylthio)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 1-isocyanato-4-(methylthio)benzene to give ethyl 5-cyano-6-[4-({[4-(methylthio)phenyl]amino}carbonyl)piperazin-1-yl]-2-(trifluoromethyl)nicotinate. Yield: 42.9 mg (87%).

¹H NMR (400 MHz, d₆-DMSO): δ 1.28 (3H, t, J=7.1 Hz), 2.40 (3H, s), 3.60-3.67 (4H, m), 3.90-3.96 (4H, m), 4.28 (2H, q, J=7.1 Hz), 7.14-7.19 (2H, m), 7.40-7.45 (2H, m), 8.55 (1H, s), 8.57 (1H, s). MS m/z: 495 (M+1).

### Example 94

### ethyl 6-{4-[(1,3-benzodioxol-5-ylamino)carbonyl]piperazin-1-yl}-5-chloronicotinate

Prepared in according to method A from ethyl 5-chloro-6-piperazin-1-ylnicotinate and 5-isocyanato-1,3-benzodioxole give ethyl 6-{4-[(1,3-benzodioxol-5-ylamino)carbonyl]piperazin-1-yl}-5-chloronicotinate. Yield: 27.9 mg (64%).

¹H NMR (400 MHz, d₆-DMSO): δ 1.28 (3H, t, J=7.1 Hz), 3.48-3.59 (8H, m), 4.28 (2H, q, J=7.1 Hz), 5.92 (2H, s), 6.75-6.84 (2H, m), 7.11-7.13 (1H, m), 8.09-8.11 (1H, m), 8.45 (1H, s), 8.66-8.68 (1H, m). MS m/z: 434 (M+1).

### Example 95

### 3-{1-{[(5-chloro-2-thienyl)amino]carbonyl}-4-[3-cyano-5-(ethoxycarbonyl)-6-(trifluoromethyl)pyridin-2-yl]piperazin-2-yl}propanoic acid

### (a) Ethyl 6-(3-(3-tert-butoxy-3-oxopropyl)-4-{[(5-chloro-2-thienyl)amino]carbonyl}piperazin-1-yl)-5-cyano-2-(trifluoromethyl)nicotinate

Ethyl 6-[3-(3-tert-butoxy-3-oxopropyl)piperazin-1-yl]-5-cyano-2-(trifluoromethyl)nicotinate (28 mg, 0.06 mmol) was dissolved in CH₂Cl₂ (1 ml) under nitrogen. A solution of 2-chloro-5-isocyanatothiophene (15 mg, 0.09 mmol) in CH₂Cl₂ (1 ml) was added. The resulting solution was stirred at room temperature over night. Water (10 ml) and CH₂Cl₂ (8 ml) was added. The phases were separated and the organic phase washed with water (10 ml), dried (MgSO₄) and evaporated in vacuo. Yield: 41 mg (quant.).

¹H NMR (400 MHz, CDCl₃): δ 1.38 (3H, t, J=7.2 Hz), 1.53 (9H, s), 1.77-1.96 (2H, m), 2.42-2.50 (2H, m), 3.35 (1H, dt, J=3.7 and 12.1 Hz), 3.58-3.74 (2H, m), 3.98-4.07 (1H, m), 4.28-4.36 (1H, m), 4.38 (2H, q, J=7.1 Hz), 4.42-4.56 (2H, m), 6.42 (1H, d, J=4.0 Hz), 6.65 (1H, d, J=4.0 Hz), 8.39 (1H, s), 9.75 (1H, s). MS m/z: 616 (M+1).

### (b) 3-{1-{[(5-Chloro-2-thienyl)amino]carbonyl}-4-[3-cyano-5-(ethoxycarbonyl)-6-(trifluoromethyl)pyridin-2-yl]piperazin-2-yl}propanoic acid

Ethyl 6-(3-(3-tert-butoxy-3-oxopropyl)-4-{[(5-chloro-2-thienyl)amino]carbonyl}piperazin-1-yl)-5-cyano-2-(trifluoromethyl)nicotinate (41 mg, 0.067 mmol) was dissolved in CH₂Cl₂ (2 ml). Trifluoroacetic acid (1 ml) was added. The resulting solution was stirred at room temperature for 1.5 h. The solvents were removed in vacuo. The residue was submitted to flash chromatography (SiO₂, CH₂Cl₂/methanol 50:1→20:1). The fractions containing the product were combined and evaporated in vacuo. The residue was dissolved in CH₂Cl₂ (20 ml) and activated carbon (0.3 g) was added. The suspension was refluxed for 10 min and filtered through Celite. The filter cake washed with CH₂Cl₂ and methanol. The filtrate was evaporated in vacuo. The residue was purified by preparative HPLC (0.1M ammonium acetate buffer/acetonitrile 80:20→60:40). The pure fractions were combined and concentrated to about 10 ml in vacuo. This suspension was extracted with CH₂Cl₂ (3×10 ml). The combined organic extracts were dried (Na₂SO₄) and evaporated in vacuo to give 3-{1-{[(5-Chloro-2-thienyl)amino]carbonyl}-4-[3-cyano-5-(ethoxycarbonyl)-6-(trifluoromethyl)pyridin-2-yl]piperazin-2-yl}propanoic acid. Yield: 3 mg (9%).

¹H NMR (400 MHz, CDCl₃): δ 1.38 (3H, t, J=7.2 Hz), 1.88-2.00 (2H, m), 2.60-2.66 (2H, m), 3.28-3.40 (1H, m), 3.57-3.68 (2H, m), 4.03-4.12 (1H, m), 4.31 (1H, dt, J=3.9 and 13.9 Hz), 4.38 (2H, q, J=7.1 Hz), 4.46-4.54 (1H, m), 4.61 (1H, d, J=14.1 Hz), 6.36 (1H, d, J=4.0 Hz), 6.62 (1H, d, J=4.0 Hz), 8.39 (1H, s), 9.14 (1H, s br). MS m/z: 560 (M+1).

### Example 96

### ethyl 5-chloro-6-(4-{[(2,4-dichlorophenyl)amino]carbonyl}piperazin-1-yl)nicotinate

Prepared in according to method A from ethyl 5-chloro-6-piperazin-1-ylnicotinate and 2,4-dichloro-1-isocyanatobenzene to give ethyl 5-chloro-6-(4-{[(2,4-dichlorophenyl)amino]carbonyl}piperazin-1-yl)nicotinate. Yield: 32.2 mg (70%).

¹H NMR (400 MHz, d₆-DMSO): δ 1.29 (3H, t, J=7.1 Hz), 3.50-3.62 (8H, m), 4.29 (2H, q, J=7.1 Hz), 7.35 (1H, dd, J₁=8.7 Hz, J₂=2.4 Hz), 7.52 (1H, d, J=8.7 Hz), 7.59 (1H, d, J=2.4 Hz), 8.10-8.12 (1H, m), 8.34 (1H, s), 8.67-8.69 (1H, m). MS m/z: 459 (M+1).

### Example 97

### ethyl 5-chloro-6-(4-{[(3-nitrophenyl)amino]carbonyl}piperazin-1-yl)nicotinate

Prepared in according to method A from ethyl 5-chloro-6-piperazin-1-ylnicotinate and 1-isocyanato-3-nitrobenzene to give ethyl 5-chloro-6-(4-{[(3-nitrophenyl)amino]carbonyl}piperazin-1-yl)nicotinate. Yield: 19.8 mg (45%).

¹H NMR (400 MHz, d₆-DMSO): δ 1.29 (3H, t, J=7.1 Hz), 3.50-3.67 (8H, m), 4.28 (2H, q, J=7.1 Hz), 7.51 (1H, t, J=8.1 Hz), 7.75-7.80 (1H, m), 7.86-7.92 (1H, m), 8.09-8.12 (1H, m), 8.45-8.49 (1H, m), 8.66-8.69 (1H, m), 9.10 (1H, s). MS m/z: 435 (M+1).

### Example 98

### ethyl 5-cyano-6-(4-{[(4-fluoro-3-nitrophenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-fluoro-4-isocyanato-2-nitrobenzene to give ethyl 5-cyano-6-(4-{[(4-fluoro-3-nitrophenyl)amino]carbonyl}piperazin-1-yl)-2-(trifluoromethyl)nicotinate. Yield: 39.7 mg (77%).

¹H NMR (400 MHz, d₆-DMSO): δ 1.29 (3H, t), 3.65-3.71 (4H, m), 3.94-3.99 (4H, m), 4.29 (2H, q), 7.45-7.52 (1H, dd), 7.83-7.89 (1H, dt), 8.35-8.39 (1H, dd), 8.57 (1H, s), 9.05 (1H, s).

### Example 99

### ethyl 5-cyano-6-[4-({[4-(dimethylamino)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 4-isocyanato-N,N-dimethylaniline to give ethyl 5-cyano-6-[4-({[4-(dimethylamino)phenyl]amino}carbonyl)piperazin-1-yl]-2-(trifluoromethyl)nicotinate. Yield: 18.3 mg (37%).

¹H NMR (400 MHz, d₆-DMSO): δ 1.29 (3H, t), 2.81 (6H, s), 3.59-3.65 (4H, m), 3.91-3.97 (4H, m), 4.29 (2H, q), 6.65 (2H, d), 7.24 (2H, d), 8.26 (1H, s), 8.56 (1H, s).

### Example 100

### ethyl 5-chloro-6-(4-{[(4,5-dimethyl-2-nitrophenyl)amino]carbonyl}piperazin-1-yl)nicotinate

Prepared in according to method A from ethyl 5-chloro-6-piperazin-1-ylnicotinate and 1-isocyanato-4,5-dimethyl-2-nitrobenzene to give ethyl 5-chloro-6-(4-{[(4,5-dimethyl-2-nitrophenyl)amino]carbonyl}piperazin-1-yl)nicotinate. Yield: 13 mg (28%).

¹H NMR (400 MHz, d₆-DMSO): δ 1.30 (3H, t), 2.22 (3H, s), 2.25 (3H, s), 3.53-3.57 (4H, m), 3.59-3.64 (4H, m), 4.30 (2H, q), 7.58 (1H, s), 7.78 (1H, s), 8.13 (1H, d), 8.69 (1H, d), 9.27 (1H, s).

### Example 101

### ethyl 5-cyano-6-(4-{[(4-methoxy-2-methylphenyl)amino]carbonyl}piperazin-1-yl)-2-(trifluoromethyl)nicotinate

Prepared in according to method A from ethyl 5-cyano-6-piperazin-1-yl-2-(trifluoromethyl)nicotinate and 1-isocyanato-4-methoxy-2-methylbenzene to give ethyl 5-cyano-6-{4-[(4-methoxy-2-methylphenyl)carbamoyl]piperazin-1-yl}-2-(trifluoromethyl)nicotinate. Yield: 29.7 mg (60%).

¹H NMR (400 MHz, d₆-DMSO): δ 1.29 (3H, t), 2.13 (3H, s) 3.60-3.65 (4H, m), 3.71 (3H, s), 3.92-3.96 (4H, m), 4.29 (2H, q), 6.67-6.72 (1H, dd), 6.76 (1H, d), 7.05 (1H, d), 7.99 (1H, s), 8.55 (1H, s).

### Example 102

### ethyl 5-chloro-6-(4-{[(2-methoxyphenyl)amino]carbonyl}piperazin-1-yl)nicotinate

Prepared in according to method A from ethyl 5-chloro-6-piperazin-1-ylnicotinate and 1-isocyanato-2-methoxybenzene to give ethyl 5-chloro-6-(4-{[(2-methoxyphenyl)amino]carbonyl}piperazin-1-yl)nicotinate. Yield: 25.5 mg (60%).

¹H NMR (400 MHz, d₆-DMSO): δ 1.30 (3H, t), 3.51-3.61 (8H, m), 3.81 (3H, s), 4.30 (2H, q), 6.83-6.91 (1H, m), 6.98-7.03 (2H, m), 7.66 (1H, d), 7.72 (1H, s), 8.11 (1H, d), 8.68 (1H, d).

### Example 103

### ethyl 6-(4-{[(4-butoxyphenyl)amino]carbonyl}piperazin-1-yl)-5-chloronicotinate

Prepared in according to method A from ethyl 5-chloro-6-piperazin-1-ylnicotinate and 1-butoxy-4-isocyanatobenzene to give ethyl 6-(4-{[(4-butoxyphenyl)amino]carbonyl}piperazin-1-yl)-5-chloronicotinate. Yield: 7.6 mg (16%) MS m/z: 461 (M+1).

### Example 104

### ethyl 6-{4-[(benzylamino)carbonyl]piperazin-1-yl}-5-chloronicotinate

Prepared in according to method A from ethyl 5-chloro-6-piperazin-1-ylnicotinate and (isocyanatomethyl)benzene to give ethyl 6-{4-[(benzylamino)carbonyl]piperazin-1-yl}-5-chloronicotinate. Yield: 25.1 mg (62%).

¹H NMR (400 MHz, d₆-DMSO): δ 1.30 (3H, t), 3.48 (8H, apparent br s), 4.24-4.33 (4H, m), 7.13-7.22 (2H, m), 7.24-7.33 (4H, m), 8.10 (1H, d), 8.66 (1H, d).

### Example 105

### ethyl 5-cyano-6-{4-[(octylamino)carbonyl]piperazin-1-yl}-2-(trifluoromethyl)nicotinate

Prepared in according to method A from ethyl 5-cyano-6-piperazin-1-yl-2-(trifluoromethyl)nicotinate and 1-isocyanatooctane to give ethyl 5-cyano-6-{4-[(octylamino)carbonyl]piperazin-1-yl}-2-(trifluoromethyl)nicotinate. Yield: 25.6 mg (53%).

¹H NMR (400 MHz, d₆-DMSO): δ 0.85 (3H, t), 1.24 (10H, apparent br s), 1.28 (3H, t), 1.35-1.45 (2H, m), 3.01 (2H, q), 3.44-3.50 (4H, m), 3.83-3.89 (4H, m), 4.28 (2H, q), 6.56 (1H, br t), 8.55 (1H, s).

### Example 106

### ethyl 5-chloro-6-(4-{[(2-phenylethyl)amino]carbonyl}piperazin-1-yl)nicotinate

Prepared in according to method A from ethyl 5-chloro-6-piperazin-1-ylnicotinate and (2-isocyanatoethyl)benzene to give ethyl 5-chloro-6-(4-{[(2-phenylethyl)amino]carbonyl}piperazin-1-yl)nicotinate. Yield: 35.2 mg (84%).

¹H NMR (400 MHz, d₆-DMSO): δ 1.30 (3H, t), 2.72 (2H, apparent t), 3.20-3.28 (2H, m), 3.43 (8H, apparent br s), 4.30 (2H, q), 6.63 (1H, t), 7.15-7.21 (3H, m), 7.25-7.30 (2H, m), 8.10 (1H, s), 8.67 (1H, d).

### Example 107

### ethyl 6-[4-(anilinocarbonyl)piperidin-1-yl]-5-chloronicotinate

### (a) 1-(3-Chloro-5-(ethoxycarbonyl)pyridin-2-yl)piperidine-4-carboxylic acid

Prepared in essentially the same way as described in Example 2a starting from ethyl 5,6-dichloronicotinate and piperidine-4-carboxylic acid (replacing the piperazine). Purification was done by flash chromatography (eluant 25% EtOAc/Hexanes to 25% EtOAc 1% AcOH/Hexanes).

### (b) ethyl 6-[4-(anilinocarbonyl)piperidin-1-yl]-5-chloronicotinate

1-(3-Chloro-5-(ethoxycarbonyl)pyridin-2-yl)piperidine-4-carboxylic acid (0.250 g, 0.80 mmol), EDCI (0.199 g, 1.04 mmol) and HOBT (0.140 g, 1.04 mmol) were suspended in DCM (5 mL) at room temperature. The reaction mixture was stirred at room temperature for 30 minutes and then aniline (0.149 g, 1.60 mmol) and DIEA (0.42 mL, 2.40 mmol) were added drop-wise. The reaction mixture was stirred at room temperature until complete consumption of the starting material was observed by HPLC analysis. The reaction mixture was diluted with DCM (20 mL) and washed with saturated NaHCO₃ (1×20 mL). The combined organics were dried (MgSO₄) and concentrated under reduced pressure to afford the crude material. Flash chromatography (gradient elution 1-2% MeOH/DCM) gave ethyl 6-[4-(anilinocarbonyl)piperidin-1-yl]-5-chloronicotinate as a solid. Yield: 0.278 g (90%).

¹H NMR (400 MHz, CDCl₃): δ 1.39 (3H, t, J=7.0 Hz), 1.98-2.10 (4H, m), 2.45-2.56 (1H, m), 2.95-3.05 (2H, m), 4.17-4.26 (2H, m), 4.37 (2H, q, J=7.0 Hz), 7.08-7.21 (2H, m), 7.30-7.38 (2H, m), 7.50-7.57 (2H, m), 8.14 (1H, s), 8.77 (1H, s). MS m/z: 388 (M+1).

### Example 108

### ethyl 5-chloro-6-(4-{[(2-ethyl-6-isopropylphenyl)amino]carbonyl}piperazin-1-yl)nicotinate

Prepared in according to method A from ethyl 5-chloro-6-piperazin-1-ylnicotinate and 1-ethyl-2-isocyanato-3-isopropylbenzene to give ethyl 5-chloro-6-(4-{[(2-ethyl-6-isopropylphenyl)amino]carbonyl}piperazin-1-yl)nicotinate. Yield: 22.5 mg (49%).

¹H NMR (400 MHz, d₆-DMSO): δ 1.07-1.15 (9H, m), 1.30 (3H, t), 2.42-2.50 (2H, m, overlapping with the signal from DMSO), 3.11 (1H, m), 3.49-3.54 (4H, m), 3.58-3.63 (4H, m), 4.30 (2H, q), 7.04-7.07 (1H, m), 7.10-7.20 (2H, m), 7.92 (1H, s), 8.12 (1H, d), 8.70 (1H, d).

### Example 109

### ethyl 5-cyano-6-[4-({[3-(methoxycarbonyl)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and methyl 3-isocyanatobenzoate to give ethyl 5-cyano-6-[4-({[3-(methoxycarbonyl)phenyl]amino}carbonyl)piperazin-1-yl]-2-(trifluoromethyl)nicotinate. Yield: 7 mg (13%).

¹H NMR (400 MHz, d₆-DMSO): δ 1.29 (3H, t), 3.65-3.70 (4H, m), 3.84 (3H, s), 3.4-3.99 (4H, m), 4.29 (2H, q), 7.38 (1H, t), 7.54 (1H, m), 7.78 (1H, m), 8.14 (1H, m), 8.57 (1H, s), 8.83 (1H, s).

### Example 110

### ethyl 5-cyano-6-[4-({[4-(difluoromethoxy)phenyl]amino}carbonyl)piperazin-1-yl]-2-(trifluoromethyl)nicotinate

Prepared in according to method A from ethyl 5-cyano-6-piperazin-1-yl-2-(trifluoromethyl)nicotinate and 1-(difluoromethoxy)-4-isocyanatobenzene to give ethyl 5-cyano-6-[4-({[4-(difluoromethoxy)phenyl]amino}carbonyl)piperazin-1-yl]-2-(trifluoromethyl)nicotinate. Yield: 31.4 mg (61%).

1.28 (3H, t), 3.61-3.67 (4H, m), 3.91-3.97 (4H, m), 4.27 (2H, q), 7.05 (2H, d), 7.09 (1H, t, OCHF₂), 7.48 (2H, d), 8.55 (1H, s), 8.63 (1H, s).

### Example 111

### ethyl 5-chloro-6-[4-({[3-fluoro-5-(trifluoromethyl)phenyl]amino}carbonyl)piperazin-1-yl]nicotinate

Prepared in according to method A from ethyl 5-chloro-6-piperazin-1-ylnicotinate and 1-fluoro-3-isocyanato-5-(trifluoromethyl)benzene to give ethyl 5-chloro-6-[4-({[3-fluoro-5-(trifluoromethyl)phenyl]amino}carbonyl)piperazin-1-yl]nicotinate. Yield: 30.3 mg (63%).

¹H NMR (400 MHz, d₆-DMSO): δ 1.30 (3H, t), 3.52-3.58 (4H, m), 3.60-3.65 (4H, m), 4.29 (2H, q), 7.16 (1H, apparent d), 7.70-7.75 (2H, m), 7.94 (1H, s), 8.11 (1H, d), 8.68 (1H, d), 9.11 (1H, s).

### Example 112

### ethyl 5-chloro-6-(4-{[(2,6-dimethoxyphenyl)amino]carbonyl}piperazin-1-yl)nicotinate

Prepared in according to method A from ethyl 5-chloro-6-piperazin-1-ylnicotinate and 2-isocyanato-1,3-dimethoxybenzene to give ethyl 5-chloro-6-(4-{[(2,6-dimethoxyphenyl)amino]carbonyl}piperazin-1-yl)nicotinate. Yield: 20.3 mg (45%).

¹H NMR (400 MHz, d₆-DMSO): δ 1.30 (3H, t), 3.48-3.57 (8H, m), 3.71 (6H, s), 4.30 (2H, q), 6.64 (2H, d), 7.14 (1H, t), 7.50 (1H, s), 8.11 (1H, d), 8.69 (1H, d).

### Example 113

### N-benzyl-1-[3-chloro-5-(5-ethyl-1,3-oxazol-2-yl)pyridin-2-yl]piperidine-4-carboxamide

### (a) 5,6-Dichloro-N-(2-hydroxybutyl)nicotinamide

5,6-Dichloronicotinic acid (20.0 g, 104 mmol), EDCI (26.0 g, 135 mmol) and HOBt (18.3 g, 135 mmol) were dissolved in DCM (500 mL) at r.t. The reaction mixture was stirred at r.t for 90 minutes and then 1-aminobutan-2-ol (15.0 g, 156 mmol) and DIPEA (54.4 mL, 313 mmol) were added. The reaction mixture was stirred at r.t for 18 h. The reaction mixture was diluted with DCM (400 mL) and the combined organics were washed with saturated NH₄Cl (2×100 mL), saturated NaHCO₃ (2×100 mL), dried (MgSO₄) and concentrated under reduced pressure to afford 5,6-dichloro-N-(2-hydroxybutyl)nicotinamide as a solid, which was used crude assuming a 100% conversion.

### (b) 5,6-Dichloro-N-(2-oxobutyl)nicotinamide

Oxalyl Chloride (16.3 mL, 187 mmol) was dissolved in DCM (500 mL) and cooled to −78° C. DMSO (26.3 mL, 374 mmol) was added drop-wise and stirred at −78° C. for 10 minutes. 5,6-Dichloro-N-(2-hydroxybutyl)nicotinamide (30 g, 94 mmol) was dissolved in DCM/DMSO (3:1) and added slowly to the solution. The solution was stirred at −78° C. for 30 minutes. TEA (65.2 mL, 467 mmol) was added to the solution and stirred for 30 minutes. The solution was warmed to r.t and stirred for 3 h. The reaction mixture was diluted with DCM (200 mL) and the combined organics were washed with water (2×200 mL), brine (2×200 mL), dried (MgSO₄) and concentrated under reduced pressure to afford 5,6-dichloro-N-(2-oxobutyl)nicotinamide as a solid, which was used crude assuming a 100% conversion.

### (c) 2,3-dichloro-5-(5-ethyl-1,3-oxazol-2-yl)pyridine

5,6-Dichloro-N-(2-oxobutyl)nicotinamide (26.7 g, 78 mmol) and POCl₃ (59.6 g, 389 mmol) were dissolved in DMF (500 mL) and heated at 90° C. for 30 minutes. The reaction mixture was poured onto ice. Solid NaHCO₃ was added in portions until the pH was raised to pH>8. The reaction mixture was diluted with water (500 mL) and the combined aqueous were washed with EtOAc (3×400 mL), dried (MgSO₄) and concentrated under reduced pressure to afford the crude product as a solid. Flash chromatography (EtOAc/hexanes, 1/9) gave 2,3-dichloro-5-(5-ethyl-1,3-oxazol-2-yl)pyridine as a solid. Yield: 7.08 g (37.5%).

¹H NMR (400 MHz, CDCl₃): δ 1.33 (2H, t, J=7.5 Hz), 2.78 (2H, q, J=7.5 Hz), 6.91 (1H, s), 8.35 (1H, d, J=1.9 Hz) 8.29 (1H, d, J=1.9 Hz). MS m/z: 244 (M+1).

### (d) 1-[3-chloro-5-(5-ethyl-1,3-oxazol-2-yl)pyridin-2-yl]piperidine-4-carboxylic acid

A suspension of 2,3-dichloro-5-(5-ethyl-1,3-oxazol-2-yl)pyridine (1.0 g, 4.11 mmol) and piperidine-4-carboxylic acid (0.797 g, 6.17 mmol) and DIPEA (1.59 g, 12.34 mmol) in DMA (20 mL) was heated to 120 degrees until the starting materials was completely consumed by HPLC analysis. The reaction mixture was concentrated. The crude material was partioned between DCM and 1 N HCl and the organics was separated dried (MgSO₄), filtered and evaporated to give 1-[3-chloro-5-(5-ethyl-1,3-oxazol-2-yl)pyridin-2-yl]piperidine-4-carboxylic acid which was used without further purification. Yield 1.27 g (92%).

### (e) N-benzyl-1-[3-chloro-5-(5-ethyl-1,3-oxazol-2-yl)pyridin-2-yl]piperidine-4-carboxamide

DIPEA (116 mg, 0.89 mmol) was added after 30 minutes to a stirred solution of 1-[3-chloro-5-(5-ethyl-1,3-oxazol-2-yl)pyridin-2-yl]piperidine-4-carboxylic acid (100 mg, 0.298 mmol), EDCI (74 mg, 0.39 mmol), HOBT (52 mg, 0.39 mmol) and bensylamine (48 mg, 0.45 mmol) at r.t. and the stirring was continued until complete consumption of starting materials was observed by HPLC analysis. The reaction mixture was diluted with DCM and washed with NH₄Cl (saturated): The combined organics were dried (MgSO4) and concentrated under reduced pressure to afford the crude product, which was purified by flash column chromatography using a gradient of EtOAc/hexanes (30%-70% EtOAc) to give N-benzyl-1-[3-chloro-5-(5-ethyl-1,3-oxazol-2-yl)pyridin-2-yl]piperidine-4-carboxamide. Yield: 91.4 mg (72%).

¹H NMR (400 MHz, CDCl₃): δ 1.50 (3H, t), 1.90-2.10 (4H, m), 2.30-3.02 (1H, m), 2.75 (q, 2H), 2.83-3.0 (2H, m), 4.05 (2H, apparent d), 4.5 (2H, d), 5.68 (1H, m), 6.80 (s, 1H), 7.20-7.40 (5H, m), 8.18 (1H, s), 8.67 (1H, s). MS m/z: 425 (M+1).

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

What is claimed is:

1. A compound of formula I or a pharmaceutically acceptable salt thereof: 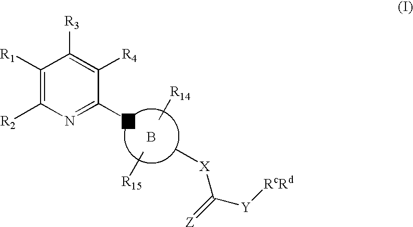
wherein:
R₁ represents R₆OC(O), R₇C(O), R₁₆SC(O), R₁₇S, R₁₈C(S) or a group gII 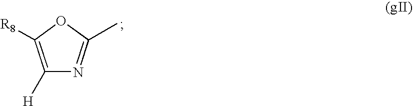
R₂ represents H, CN, halogen, NO₂, (C₁-C₁₂)alkyl optionally interrupted by oxygen and/or optionally substituted by OH, aryl, cycloalkyl, heterocyclyl or one or more halogen atoms; or
R₂ represents (C₁-C₁₂)alkoxy optionally substituted by one or more halogen atoms; or
R₂ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine; or
R₁ and R₂, together (with two carbon atoms of the pyridine ring), may form a 5-membered or 6-membered cyclic lactone;
R₃ represents H, CN, NO₂, halogen, (C₁-C₁₂)alkyl optionally interrupted by oxygen and/or optionally substituted by OH, aryl, cycloalkyl, heterocyclyl or one or more halogen atoms; or
R₃ represents (C₁-C₁₂)alkoxy optionally substituted by one or more halogen atoms; or
R₃ represents (C₃-C₆)cycloalkyl, hydroxy(C₁-C₁₂)alkyl, (C₁-C₁₂)alkylC(O), (C₁-C₁₂)alkylthiOC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;
R₄ represents a halogen atom or is CN;
Z represents O or S;
R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₁₂)alkyl, aryl or heterocyclyl;
R₇ represents (C₁-C₁₂)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₁₂)alkyl, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O), (C₁-C₁₂)alkoxyC(O) or R^{a(15)} and R^{b(15)} together with the nitrogen atom represent piperidine, pyrrolidine, azetidine or aziridine;
R₁₆ represents (C₁-C₁₂)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;
R₁₇ represents (C₁-C₁₂)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;
Y represents imino (—NH—) or is absent;
R^{c} represents imino or (C₁-C₄)alkyleneimino or an unsubstituted or monosubstituted or polysubstituted (C₁-C₄)alkylene group or (C₁-C₄)oxoalkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogen, hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine;
R₁₉ represents H or (C₁-C₄)alkyl;
R^{d} represents (C₁-C₁₂)alkyl, (C₃-C₈)cycloalkyl, aryl or heterocyclyl, and anyone of these groups optionally substituted with one or more halogen atoms and/or one or more of the following groups: OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, aryloxy, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; or
X represents a group (—CH₂—)ₙ wherein n=2-6, which optionally is unsaturated and/or substituted by one or more substituent chosen among halogen, hydroxyl or (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections);
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

2. A compound according to claim 1 wherein:
R₂ represents H, CN, NO₂, (C₁-C₆)alkyl optionally interrupted by oxygen and/or optionally substituted by OH, aryl, cycloalkyl, heterocyclyl or one or more halogen atoms; or
R₂ represents (C₁-C₆)alkoxy optionally substituted by one or more halogen atoms; or
R₂ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine; or
R₁ and R₂ together (with two carbons from the pyridine ring) may form a 5-membered or 6-membered cyclic lactone;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₆)alkyl, aryl or heterocyclyl;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ represents (C₁-C₆)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;
R₁₇ represents (C₁-C₆)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₆)alkyl, (C₁-C₆)alkoxy, (C₃-C₆)cycloalkoxy, aryl or heterocyclyl; and
R^{d} represents (C₁-C₁₀)alkyl, (C₃-C₈)cycloalkyl, aryl or heterocyclyl, and anyone of these groups optionally substituted with one or more halogen atoms and/or one or more of the following groups: OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, aryloxy, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

3. A compound according to claim 2 wherein:
R₁ represents R₆OC(O) or a group gII, 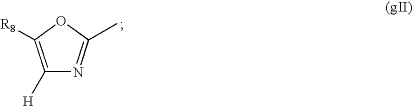
R₂ represents H, CN, NO₂, (C₁-C₆)alkyl optionally interrupted by oxygen and/or optionally substituted by OH, aryl, cycloalkyl, heterocyclyl or one or more halogen atoms; or
R₂ represents (C₁-C₆)alkoxy optionally substituted by one or more halogen atoms; or
R₂ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthiOC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl or heterocyclyl;
R₁₄ represents H or OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H or OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R^{d} represents (C₁-C₁₀)alkyl, (C₃-C₈)cycloalkyl, aryl or heterocyclyl, and anyone of these groups optionally substituted with one or more halogen atoms and/or one or more of the following groups, OH, CN, NO₂, (C₁-C₆)alkyl, (C₁-C₆)alkoxy, halosubstituted (C₁-C₆)alkyl, (C₃-C₆)cycloalkyl, aryl, aryloxy, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

4. A compound according to claim 1 wherein:
R₁ represents R₆OC(O) or a group gII 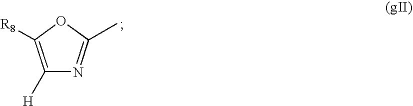
R₂ represents H or (C₁-C₆)alkyl optionally interrupted by oxygen and/or optionally substituted by OH, aryl, cycloalkyl, heterocyclyl or one or more halogen atoms;
R₃ represents H;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms;
R₈ represents H, (C₁-C₆)alkyl optionally interrupted by oxygen, and/or optionally substituted by aryl, cycloalkyl, heterocyclyl or one or more halogen atoms;
R₁₄ represents H or OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl;
R₁₅ represents H;
R^{d} represents (C₁-C₁₀)alkyl, (C₃-C₆)cycloalkyl, aryl or heterocyclyl, and anyone of these groups optionally substituted with one or more halogen atoms and/or one or more of the following groups: CN, NO₂, (C₁-C₆)alkyl, (C₁-C₆)alkoxy, (C₁-C₆)alkylthio, halosubstituted (C₁-C₆)alkyl, aryl and aryloxy;
X represents a single bond, imino (—NH—), methylene (—CH₂—) or iminomethylene (—CH₂—NH—); and
B is a monocyclic, 4 to 7-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections);
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

5. A compound according to claim 1 wherein:
R₁ is ethoxycarbonyl;
R₂ is chosen from a group consisting of H, methyl and trifluoromethyl;
R₃ is H;
R₄ is chosen from a group consisting of bromo, chloro and cyano;
Z is O or S;
R₆ is ethyl;
R₈ is ethyl;
R₁₄ is H or carboxyethyl;
R₁₅ is H;
R^{c} is chosen from being absent or from a group consisting of methylene (—CH₂—), methylmethylene (—CH(CH₃)—), dimethylmethylene (—C(CH₃)₂—), ethylene (—CH₂CH₂—), imino (—NH—), carbonyl (—CO—) and 1-carboxy-ethylene;
R^{d} is chosen from a group consisting of n-octyl, 2-phenyl-cyclopropyl, phenyl, 2-methylphenyl, 3-methoxycarbonyl-phenyl, 2-methoxy-5-methyl-phenyl, 4-methoxy-2-methyl-phenyl, 3-methylphenyl, 4-methylphenyl, 2-methoxyphenyl, 3-methoxyphenyl, 4-methoxyphenyl, 4-butoxy-phenyl, 2,6-dimethoxy-phenyl, 3-thiomethyl-phenyl, 4-thiomethyl-phenyl, 2-ethyl-6-isopropyl-phenyl, 2-fluoro-5-methyl-phenyl, 3-fluoro-5-(trifluoromethyl)-phenyl, 3-fluorophenyl, 4-fluorophenyl, 4-fluoro-3-nitro-phenyl, 3,4-difluorophenyl, (difluoromethoxy)-phenyl, 2-chlorophenyl, 3-chlorophenyl, 4-chlorophenyl, 5-chloro-2,4-dimethoxy-phenyl, 2-bromophenyl, 3-bromophenyl, 4-bromophenyl, 3-cyanophenyl, 2-ethoxyphenyl, 4-ethoxyphenyl, 3-nitrophenyl, 2-methyl-3-nitrophenyl, 3,5-dinitrophenyl, 2,4-dichlorophenyl, 3,4-dichlorophenyl, 3,5-dichlorophenyl, 2,4,5-trichloro-phenyl, 4,5-dimethyl-2-nitro-phenyl, 4-(dimethylamino)-phenyl, 2-isopropylphenyl, 4-isopropylphenyl, 3-isopropenylphenyl, 2-phenyl-phenyl, 4-phenoxy-phenyl, 2-naphtyl, 3-naphtyl, 2-thienyl, 5-chloro-2-thienyl, and 1,3-benzodioxol-5-yl;
X represents a single bond, imino (—NH—), methylene (—CH₂—) or iminomethylene (—CH₂—NH—); and
B is chosen from the group consisting of 1,4-diazepan-1-ylene, 4-piperazin-1-ylene, 4-piperidin-1-ylene, and 3-azetidin-1-ylene, and the substituents R₁₄ and R₁₅ are connected to the B ring/ring system, in such a way that no quarternary ammonium compounds are formed (by these connections);
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

6. A compound according to claim 1 which is of the formula (Ia): 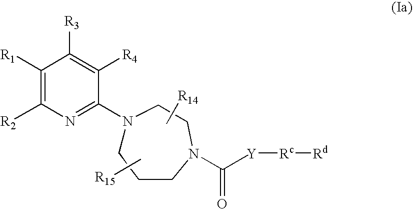
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

7. A compound according to claim 1 which is of the formula (Ib): 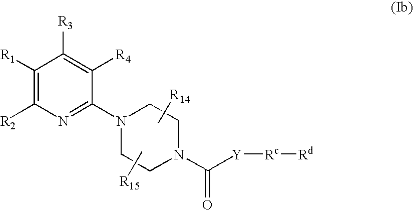
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

8. A compound according to claim 1 which is of the formula (Ic): 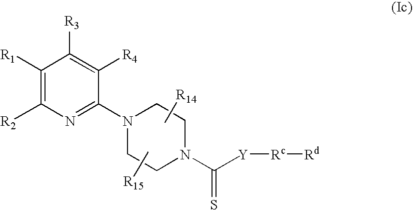
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

9. A compound according to claim 1 which is of the formula (Id): 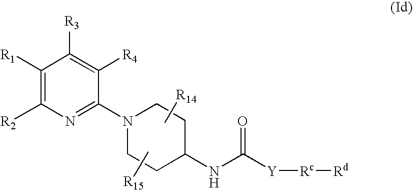
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

10. A compound according to claim 1 which is of the formula (Ie): 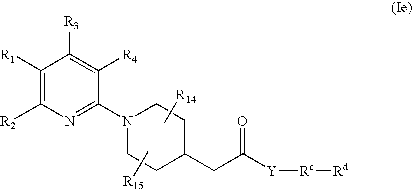
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

11. A compound according to claim 1 which is of the formula (If): 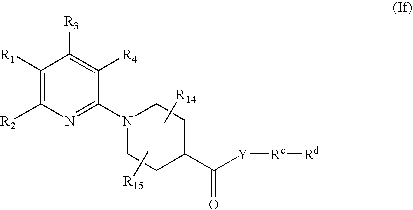
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

12. A compound according to claim 1 which is of the formula (Ig): 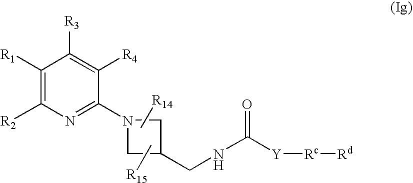
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

13. A compound according to claim 1 which is of the formula (Ih): 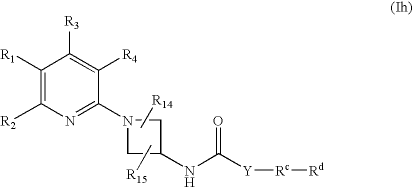
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

14. A compound according to claim 1 which is of the formula (Ii): 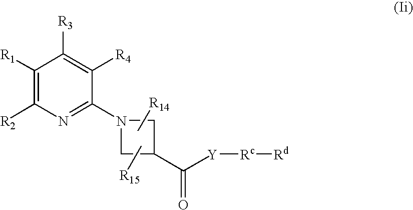
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

15. A compound according to claim 1 wherein R₁ represents R₆OC(O) with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

16. A compound according to claim 1 wherein R₁ represents a group gII

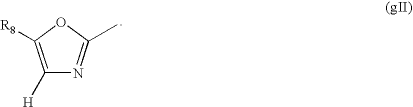

17. A compound according to claim 15 which is of the formula (Iaa): 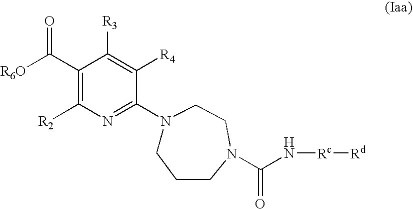
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

18. A compound according to claim 15 which is of the formula (Ibb): 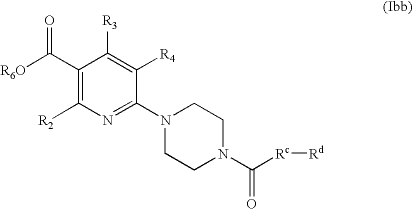
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

19. A compound according to claim 15 which is of the formula (Ibc): 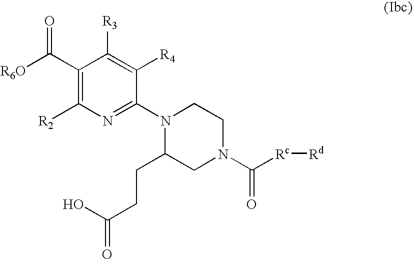
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

20. A compound according to claim 15 which is of the formula (Ibd): 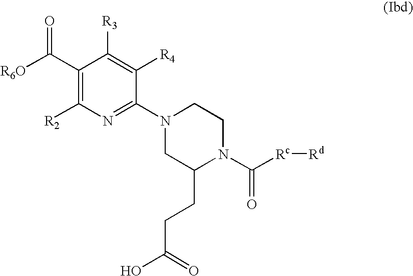
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

21. A compound according to claim 15 which is of the formula (Ibe): 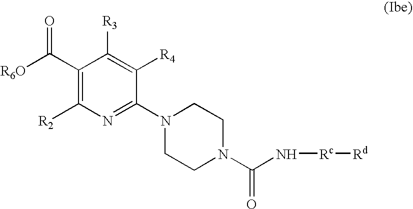
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

22. A compound according to claim 15 which is of the formula (Icc): 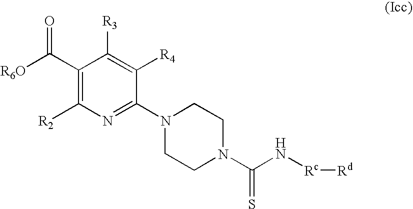
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

23. A compound according to claim 15 which is of the formula (Idd): 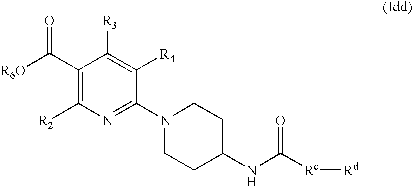
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

24. A compound according to claim 15 which is of the formula (Iee): 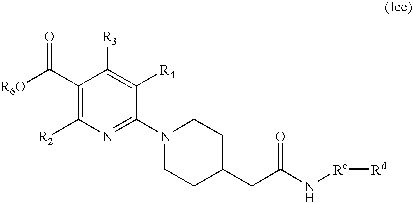
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

25. A compound according to claim 15 which is of the formula (Ief): 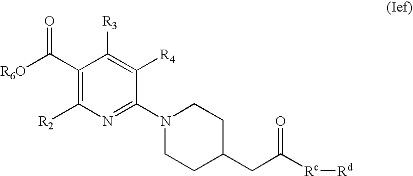
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

26. A compound according to claim 15 which is of the formula (Iff): 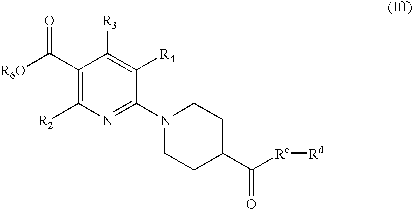
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

27. A compound according to claim 15 which is of the formula (Igg): 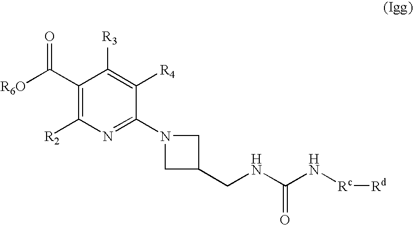
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

28. A compound according to claim 15 which is of the formula (Igh): 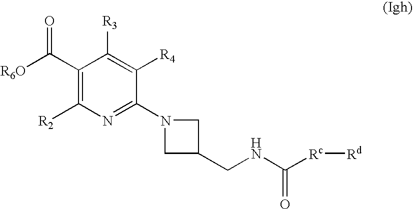
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

29. A compound according to claim 15 which is of the formula (Ihh): 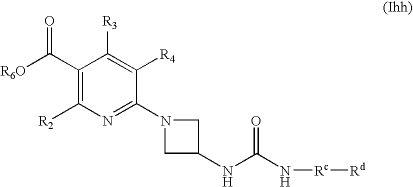
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

30. A compound according to claim 15 which is of the formula (Ihi): 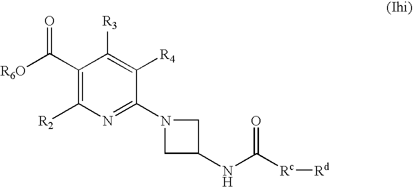
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

31. A compound according to claim 15 which is of the formula (Iii): 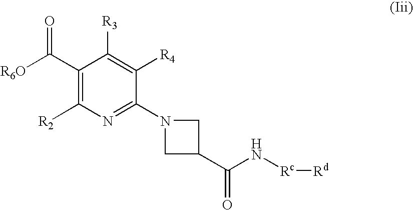
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

32. A compound according to claim 16 which is of the formula (Ijj): 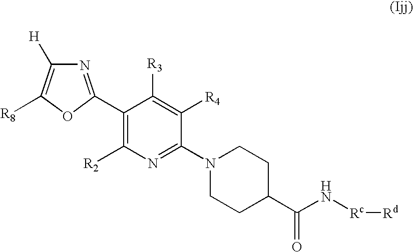
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

33. A compound according to claim 1 selected from:
ethyl 6-[4-(anilinocarbonyl)piperazin-1-yl]-5-chloronicotinate;
ethyl 6-[4-(anilinocarbonyl)piperazin-1-yl]-5-bromonicotinate;
3-{4-(anilinocarbonyl)-1-[3-chloro-5-(ethoxycarbonyl)pyridin-2-yl]piperazin-2-yl}propanoic acid;
ethyl 6-[4-(anilinocarbonyl)piperazin-1-yl]-5-cyanonicotinate;
ethyl 5-chloro-6-(4-{[(3,4-dichlorophenyl)amino]carbonyl}piperazin-1-yl)nicotinate;
ethyl 5-chloro-6-(4-{[(3,4-dichlorobenzyl)amino]carbonyl}piperazin-1-yl)nicotinate;
ethyl 5-chloro-6-(4-{[(2-methylbenzyl)amino]carbonyl}piperazin-1-yl)nicotinate;
ethyl 5-chloro-6-(4-{[(4-fluorobenzyl)amino]carbonyl}piperazin-1-yl)nicotinate;
ethyl 5-chloro-6-(4-{[(3-methylbenzyl)amino]carbonyl}piperazin-1-yl)nicotinate;
ethyl 5-chloro-6-(4-{[(4-methylbenzyl)amino]carbonyl}piperazin-1-yl)nicotinate;
ethyl 5-chloro-6-(4-{[(3-methoxyphenyl)amino]carbonyl}piperazin-1-yl)nicotinate;
ethyl 5-chloro-6-{4-[(2-naphthylamino)carbonyl]piperazin-1-yl}nicotinate;
ethyl 6-(4-{[(3-bromophenyl)amino]carbonyl}piperazin-1-yl)-5-chloronicotinate;
ethyl 5-chloro-6-[4-({[4-(methylthio)phenyl]amino}carbonyl)piperazin-1-yl]nicotinate;
ethyl 5-chloro-6-[4-({[3-(methylthio)phenyl]amino}carbonyl)piperazin-1-yl]nicotinate;
ethyl 5-chloro-6-(4-{[(3,5-dinitrophenyl)amino]carbonyl}piperazin-1-yl)nicotinate;
ethyl 5-chloro-6-(4-{[(2-methoxy-5-methylphenyl)amino]carbonyl}piperazin-1-yl)nicotinate;
ethyl 5-chloro-6-(4-{[(3-methylphenyl)amino]carbonyl}piperazin-1-yl)nicotinate;
ethyl 5-chloro-6-(4-{[(4-chlorophenyl)amino]carbonyl}piperazin-1-yl)nicotinate;
ethyl 5-chloro-6-(4-{[(3,5-dichlorophenyl)amino]carbonyl}piperazin-1-yl)nicotinate;
ethyl 5-chloro-6-(4-{[(2-isopropylphenyl)amino]carbonyl}piperazin-1-yl)nicotinate;
ethyl 5-chloro-6-[4-({[(1S)-1-phenylethyl]amino}carbonyl)piperazin-1-yl]nicotinate;
ethyl 5-chloro-6-[4-({[(1S)-1-(1-naphthyl)ethyl]amino}carbonyl)piperazin-1-yl]nicotinate;
ethyl 5-chloro-6-{4-[(1-naphthylamino)carbonyl]piperazin-1-yl}nicotinate;
ethyl 5-chloro-6-(4-{[(4-methylphenyl)amino]carbonyl}piperazin-1-yl)nicotinate;
ethyl 5-chloro-6-(4-{[(2-methylphenyl)amino]carbonyl}piperazin-1-yl)nicotinate;
ethyl 5-cyano-6-(4-{[(2,6-dimethoxyphenyl)amino]carbonyl}piperazin-1-yl)-2-(trifluoromethyl)nicotinate;
ethyl 5-cyano-6-(4-{[(2-methoxy-5-methylphenyl)amino]carbonyl}piperazin-1-yl)-2-(trifluoromethyl)nicotinate;
ethyl 5-cyano-6-(4-{[(2-isopropylphenyl)amino]carbonyl}piperazin-1-yl)-2-(trifluoromethyl)nicotinate;
ethyl 5-cyano-6-(4-{[(4-methylphenyl)amino]carbonyl}piperazin-1-yl)-2-(trifluoromethyl)nicotinate;
ethyl 5-cyano-6-(4-{[(3-methylphenyl)amino]carbonyl}piperazin-1-yl)-2-(trifluoromethyl)nicotinate;
ethyl 5-cyano-6-[4-({[(1S)-1-phenylethyl]amino}carbonyl)piperazin-1-yl]-2-(trifluoromethyl)nicotinate;
ethyl 5-cyano-6-(4-{[(2-ethoxyphenyl)amino]carbonyl}piperazin-1-yl)-2-(trifluoromethyl)nicotinate;
ethyl 6-(4-{[(2-chlorophenyl)amino]carbonyl}piperazin-1-yl)-5-cyano-2-(trifluoromethyl)nicotinate;
ethyl 5-cyano-6-(4-{[(2-methylbenzyl)amino]carbonyl}piperazin-1-yl)-2-(trifluoromethyl)nicotinate;
ethyl 6-(4-{[(2-chlorobenzyl)amino]carbonyl}piperazin-1-yl)-5-cyano-2-(trifluoromethyl)nicotinate;
ethyl 5-cyano-6-(4-{[(4-fluorobenzyl)amino]carbonyl}piperazin-1-yl)-2-(trifluoromethyl)nicotinate;
ethyl 5-cyano-6-[4-({[(1R,2R)-2-phenylcyclopropyl]amino}carbonyl)piperazin-1-yl]-2-(trifluoromethyl)nicotinate;
ethyl 5-cyano-6-(4-{[(3-methylbenzyl)amino]carbonyl}piperazin-1-yl)-2-(trifluoromethyl)nicotinate;
ethyl 5-cyano-6-(4-{[(4-methylbenzyl)amino]carbonyl}piperazin-1-yl)-2-(trifluoromethyl)nicotinate;
ethyl 5-cyano-6-(4-{[(3,4-dichlorobenzyl)amino]carbonyl}piperazin-1-yl)-2-(trifluoromethyl)nicotinate;
ethyl 5-cyano-6-(4-{[(3-methoxyphenyl)amino]carbonyl}piperazin-1-yl)-2-(trifluoromethyl)nicotinate;
ethyl 5-cyano-6-(4-{[(2-fluoro-5-methylphenyl)amino]carbonyl}piperazin-1-yl)-2-(trifluoromethyl)nicotinate;
ethyl 6-(4-{[(3-chlorophenyl)amino]carbonyl}piperazin-1-yl)-5-cyano-2-(trifluoromethyl)nicotinate;
ethyl 5-cyano-6-[4-({[2-(2-thienyl)ethyl]amino}carbonyl)piperazin-1-yl]-2-(trifluoromethyl)nicotinate;
ethyl 5-cyano-6-(4-{[(3-cyanophenyl)amino]carbonyl}piperazin-1-yl)-2-(trifluoromethyl)nicotinate;
ethyl 5-cyano-6-(4-{[(2-methoxyphenyl)amino]carbonyl}piperazin-1-yl)-2-(trifluoromethyl)nicotinate;
ethyl 6-{4-[(benzylamino)carbonyl]piperazin-1-yl}-5-cyano-2-(trifluoromethyl)nicotinate;
ethyl 6-(4-{[(5-chloro-2,4-dimethoxyphenyl)amino]carbonyl}piperazin-1-yl)-5-cyano-2-(trifluoromethyl)nicotinate;
ethyl 5-cyano-6-(4-{[(3-nitrophenyl)amino]carbonyl}piperazin-1-yl)-2-(trifluoromethyl)nicotinate;
ethyl 5-cyano-6-[4-({[3-fluoro-5-(trifluoromethyl)phenyl]amino}carbonyl)piperazin-1-yl]-2-(trifluoromethyl)nicotinate;
ethyl 5-cyano-6-[4-({[3-(methylthio)phenyl]amino}carbonyl)piperazin-1-yl]-2-(trifluoromethyl)nicotinate;
ethyl 5-cyano-6-(4-{[(3-fluorobenzyl)amino]carbonyl}piperazin-1-yl)-2-(trifluoromethyl)nicotinate;
ethyl 5-cyano-6-{4-[(2-naphthylamino)carbonyl]piperazin-1-yl}-2-(trifluoromethyl)nicotinate;
ethyl 6-(4-{[(3-bromophenyl)amino]carbonyl}piperazin-1-yl)-5-cyano-2-(trifluoromethyl)nicotinate;
ethyl 6-(4-{[(4-bromophenyl)amino]carbonyl}piperazin-1-yl)-5-cyano-2-(trifluoromethyl)nicotinate;
ethyl 6-(4-{[(2-bromophenyl)amino]carbonyl}piperazin-1-yl)-5-chloronicotinate;
ethyl 5-chloro-6-[4-({[1-(3-isopropenylphenyl)-1-methylethyl]amino}carbonyl)piperazin-1-yl]nicotinate;
ethyl 5-chloro-6-(4-{[(2-methyl-3-nitrophenyl)amino]carbonyl}piperazin-1-yl)nicotinate;
ethyl 5-chloro-6-{4-[(2-thienylamino)carbonyl]piperazin-1-yl}nicotinate;
ethyl 5-chloro-6-(4-{[(3-chlorophenyl)amino]carbonyl}piperazin-1-yl)nicotinate;
ethyl 5-cyano-6-(4-{[(3,5-dichlorophenyl)amino]carbonyl}piperazin-1-yl)-2-(trifluoromethyl)nicotinate;
ethyl 5-cyano-6-(4-{[(2-methyl-3-nitrophenyl)amino]carbonyl}piperazin-1-yl)-2-(trifluoromethyl)nicotinate;
ethyl 6-{4-[(biphenyl-2-ylamino)carbonyl]piperazin-1-yl}-5-cyano-2-(trifluoromethyl)nicotinate;
ethyl 5-cyano-6-(4-{[(3,4-dichlorophenyl)amino]carbonyl}piperazin-1-yl)-2-(trifluoromethyl)nicotinate;
ethyl 5-cyano-6-[4-({[1-(3-isopropenylphenyl)-1-methylethyl]amino}carbonyl)piperazin-1-yl]-2-(trifluoromethyl)nicotinate;
ethyl 5-cyano-6-(4-{[(4-phenoxyphenyl)amino]carbonyl}piperazin-1-yl)-2-(trifluoromethyl)nicotinate;
ethyl 5-cyano-6-(4-{[(4-methoxybenzyl)amino]carbonyl}piperazin-1-yl)-2-(trifluoromethyl)nicotinate;
3-{1-(anilinocarbonyl)-4-[3-cyano-5-(ethoxycarbonyl)-6-(trifluoromethyl)pyridin-2-yl]piperazin-2-yl}propanoic acid;
ethyl 6-{4-[(anilinocarbonyl)amino]piperidin-1-yl}-5-chloronicotinate;
ethyl 6-{3-[(anilinocarbonyl)amino]azetidin-1-yl}-5-chloronicotinate;
ethyl 6-(3-{[(anilinocarbonyl)amino]methyl}azetidin-1-yl)-5-cyano-2-methylnicotinate;
ethyl 6-[3-({[(benzylamino)carbonyl]amino}methyl)azetidin-1-yl]-5-cyano-2-methylnicotinate;
ethyl 6-{3-[(anilinocarbonyl)amino]azetidin-1-yl}-5-cyano-2-methylnicotinate;
ethyl 6-(3-{[(benzylamino)carbonyl]amino}azetidin-1-yl)-5-cyano-2-methylnicotinate;
ethyl 6-{4-[(benzoylamino)carbonothioyl]piperazin-1-yl}-5-chloronicotinate;
ethyl 5-cyano-2-methyl-6-(3-{[(phenylacetyl)amino]methyl}azetidin-1-yl)nicotinate;
ethyl 6-[4-(2-anilino-2-oxoethyl)piperidin-1-yl]-5-cyano-2-methylnicotinate;
ethyl 6-{4-[2-(benzylamino)-2-oxoethyl]piperidin-1-yl}-5-cyano-2-methylnicotinate;
phenylalanine, N-[[1-[3-cyano-5-(ethoxycarbonyl)-6-methyl-2-pyridinyl]-3-azetidinyl]carbonyl]-ethyl 5-chloro-6-(4-{[(2,4,5-trichlorophenyl)amino]carbonyl}piperazin-1-yl)nicotinate;
ethyl 6-{4-[(1,3-benzodioxol-5-ylamino)carbonyl]piperazin-1-yl}-5-cyano-2-(trifluoromethyl)nicotinate;
ethyl 5-cyano-6-(4-{[(4-isopropylphenyl)amino]carbonyl}piperazin-1-yl)-2-(trifluoromethyl)nicotinate;
ethyl 5-cyano-6-(4-{[(2-phenylethyl)amino]carbonyl}piperazin-1-yl)-2-(trifluoromethyl)nicotinate;
ethyl 6-{4-[(benzylamino)carbonyl]-1,4-diazepan-1-yl}-5-cyano-2-methylnicotinate;
ethyl 5-chloro-6-[4-({[(1R,2R)-2-phenylcyclopropyl]amino}carbonyl)piperazin-1-yl]nicotinate;
ethyl 5-cyano-6-(4-{[(3,4-difluorophenyl)amino]carbonyl}piperazin-1-yl)-2-(trifluoromethyl)nicotinate;
ethyl 5-cyano-6-(4-{[(2-methylphenyl)amino]carbonyl}piperazin-1-yl)-2-(trifluoromethyl)nicotinate;
ethyl 5-cyano-6-(4-{[(4-ethoxyphenyl)amino]carbonyl}piperazin-1-yl)-2-(trifluoromethyl)nicotinate;
ethyl 5-cyano-6-[4-({[4-(methylthio)phenyl]amino}carbonyl)piperazin-1-yl]-2-(trifluoromethyl)nicotinate;
ethyl 6-{4-[(1,3-benzodioxol-5-ylamino)carbonyl]piperazin-1-yl}-5-chloronicotinate;
3-{1-{[(5-chloro-2-thienyl)amino]carbonyl}-4-[3-cyano-5-(ethoxycarbonyl)-6-(trifluoromethyl)pyridin-2-yl]piperazin-2-yl}propanoic acid;
ethyl 5-chloro-6-(4-{[(2,4-dichlorophenyl)amino]carbonyl}piperazin-1-yl)nicotinate;
ethyl 5-chloro-6-(4-{[(3-nitrophenyl)amino]carbonyl}piperazin-1-yl)nicotinate;
ethyl 5-cyano-6-(4-{[(4-fluoro-3-nitrophenyl)amino]carbonyl}piperazin-1-yl)-2-(trifluoromethyl)nicotinate;
ethyl 5-cyano-6-[4-({[4-(dimethylamino)phenyl]amino}carbonyl)piperazin-1-yl]-2-(trifluoromethyl)nicotinate;
ethyl 5-chloro-6-(4-{[(4,5-dimethyl-2-nitrophenyl)amino]carbonyl}piperazin-1-yl)nicotinate;
ethyl 5-cyano-6-(4-{[(4-methoxy-2-methylphenyl)amino]carbonyl}piperazin-1-yl)-2-(trifluoromethyl)nicotinate;
ethyl 5-chloro-6-(4-{[(2-methoxyphenyl)amino]carbonyl}piperazin-1-yl)nicotinate;
ethyl 6-(4-{[(4-butoxyphenyl)amino]carbonyl}piperazin-1-yl)-5-chloronicotinate;
ethyl 6-{4-[(benzylamino)carbonyl]piperazin-1-yl}-5-chloronicotinate;
ethyl 5-cyano-6-{4-[(octylamino)carbonyl]piperazin-1-yl}-2-(trifluoromethyl)nicotinate;
ethyl 5-chloro-6-(4-{[(2-phenylethyl)amino]carbonyl}piperazin-1-yl)nicotinate;
ethyl 6-[4-(anilinocarbonyl)piperidin-1-yl]-5-chloronicotinate;
ethyl 5-chloro-6-(4-{[(2-ethyl-6-isopropylphenyl)amino]carbonyl}piperazin-1-yl)nicotinate;
ethyl 5-cyano-6-[4-({[3-(methoxycarbonyl)phenyl]amino}carbonyl)piperazin-1-yl]-2-(trifluoromethyl)nicotinate;
ethyl 5-cyano-6-[4-({[4-(difluoromethoxy)phenyl]amino}carbonyl)piperazin-1-yl]-2-(trifluoromethyl)nicotinate;
ethyl 5-chloro-6-[4-({[3-fluoro-5-(trifluoromethyl)phenyl]amino}carbonyl)piperazin-1-yl]nicotinate;
ethyl 5-chloro-6-(4-{[(2,6-dimethoxyphenyl)amino]carbonyl}piperazin-1-yl)nicotinate; and
N-benzyl-1-[3-chloro-5-(5-ethyl-1,3-oxazol-2-yl)pyridin-2-yl]piperidine-4-carboxamide;
and pharmaceutically acceptable salts thereof.

34. A compound according to claim 1 wherein:
R₁ is R₆OC(O);
Z is O (oxygen);
X represents imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; or
X represents a group (—CH₂—)ₙ wherein n=2-6, which optionally is unsaturated and/or substituted by one or more substituent chosen among halogen, hydroxyl or (C₁-C₆)alkyl; and
Y represents imino (—NH—) or is absent;
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

35. A compound according to claim 1 wherein:
R₁ represents R₇C(O), R₁₆SC(O), R₁₇S, R₁₈C(S) or a group gII, 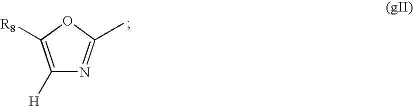
Z is O;
X represents a single bond; and
Y represents imino (—NH—) or is absent;
with the proviso that the compound or the pharmaceutically acceptable salt thereof is not:
3-pyridinecarboxylic acid, 5-cyano-6-[4-[[(1,1-dimethylethyl)amino]carbonyl]-1-piperazinyl]-2-(trifluoromethyl)-, ethyl ester or
ethyl 6-(4-{[(4-chlorophenyl)amino]carbonyl}piperazin-1-yl)-5-cyano-2-(trifluoromethyl)nicotinate or
ethyl 6-[4-(anilinocarbonyl)piperazin-1-yl]-5-cyano-2-(trifluoromethyl)nicotinate or
ethyl 5-cyano-2-(trifluoromethyl)-6-(4-{[3-(trifluoromethyl)phenyl]carbamoyl}piperazin-1-yl)nicotinate or
ethyl 6-{4-[(4-tert-butylphenyl)carbamoyl]piperidin-1-yl}-5-chloronicotinate.

36. A pharmaceutical composition comprising a compound according to claims 1 in combination with a pharmaceutically acceptable adjuvant, diluent and/or carrier.

37. A method of treatment of a platelet aggregation disorder comprising administering to a patient suffering from such a disorder a therapeutically effective amount of a compound according to claim 1 or of 3-pyridinecarboxylic acid, 5-cyano-6-[4-[[(1,1-dimethylethyl)amino]carbonyl]-1-piperazinyl]-2-(trifluoromethyl)-, ethyl ester or ethyl 6-(4-{[(4-chlorophenyl)amino]carbonyl}piperazin-1-yl)-5-cyano-2-(trifluoromethyl)nicotinate or of ethyl 6-[4-(anilinocarbonyl)piperazin-1-yl]-5-cyano-2-(trifluoromethyl)nicotinate or of ethyl 5-cyano-2-(trifluoromethyl)-6-(4-{[3-(trifluoromethyl)phenyl]carbamoyl}piperazin-1-yl)nicotinate or ethyl 6-{4-[(4-tert-butylphenyl)carbamoyl]piperidin-1-yl}-5-chloronicotinate.

38. A method of inhibition of the P2Y₁₂ receptor in a cell comprising administering to a patient having such receptor an effective amount of a compound according to claim 1 or of 3-pyridinecarboxylic acid, 5-cyano-6-[4-[[(1,1-dimethylethyl)amino]carbonyl]-1-piperazinyl]-2-(trifluoromethyl)-, ethyl ester or ethyl 6-(4-{[(4-chlorophenyl)amino]carbonyl}piperazin-1-yl)-5-cyano-2-(trifluoromethyl)nicotinate or of ethyl 6-[4-(anilinocarbonyl)piperazin-1-yl]-5-cyano-2-(trifluoromethyl)nicotinate or of ethyl 5-cyano-2-(trifluoromethyl)-6-(4-{[3-(trifluoromethyl)phenyl]carbamoyl}piperazin-1-yl)nicotinate or ethyl 6-{4-[(4-tert-butylphenyl)carbamoyl]piperidin-1-yl}-5-chloronicotinate.
